SEQUENCE LISTING (87 sequences)

SEQ ID NO: 1 - protein, 91 aa, Leavenworthia sp.
AKSVWLTSFISYLMISMLISTVIPKPHNSGVPSRKPSSTCGQQKGDESCKRLCLEIKGYLSGKCKSYGNGKFCECCRVRPCLVAHLMFPFK

SEQ ID NO: 2 - protein, 95 aa, Leavenworthia sp.
MAKSVRLISFIIYLMINMLIFAVNPKPHSSQVPPIPDPGKHRRCFLVRSKTLTSTCVKQKCYKHCIEERYVDGKCESSPKGNLCWCSIKCLKLIV

SEQ ID NO: 3 - protein, 45 aa, Leavenworthia sp.
MAKSVRLISFIIYLTINMLIFAVNPKPHSSQVPPIPDPGKHRRCF

SEQ ID NO: 4 - protein, 61 aa, Leavenworthia sp.
MAKQVSLVNFISYLMITMLISAAKKPHSLAVPLIVDRDKNNGCFGVPSKKNNLYSMQKTNG

SEQ ID NO: 5 - protein, 802 aa, Leavenworthia sp.
MTTLNNSYTFFQLFLLLVSFGVSLSINGFSLTARESVKLSEYKRSIVSPGEIFELGLFKAATRWTDMDGWYLGIWYKRLPTLVVWIANRDYPLSNSTATLKISNNNLFLDDDQSGLPVWNTNVINQINIEEPSVAELLDNGNFVLRYSNSKSFQWQSFDYPTDFLLPGMKLGWDRTKNLNKTLTSWASLTEPTSGSYVFAIENWTVSHGLLYSNGQLEFRTGPLYRNIVNITETEDEISHSLNITPNVSSTSLLQLTTAGTLQLTEFIGGERHFLFLFPLDRCDFYNKCGENSYCITNETCVCIAGFQPGGQYAVGLTKSKPRCLRKSNLSCLEKEFKKIRNVKLPDTQYAIADTKVGLEECEKRCLMNCNCTAFANTDMGNGESGCVMWTGDLIDVRSYNTDEGQDLYVKLPADDLRGKRNVNIKTIIGSIIGGLGLLGLVCYWLVITRNRSKTNSPSDSSQVFEDWGSICMDYDVIATATGNFSDSNTLGKGGFGTVYKGQLPDGHKIAVKKMTADSKGGLTGLGNEINLIAKVQHSNLIRLLGFCSTSHPDHNLLVYEYVENSSLDTYIFDTTGQYALDWEMRFEIIKGIVRGLIYLHQDSRFRIIHLDLKPNNILLDKDMIPKISDFGLAQTLERNATKGFVETAVGTFGYIAPELRNDNVYSVKSDVYSFGVMLLEIVSGKKNMEYFKNFDGTSLLRYIWDSWSKGKVLEIVDPVLKDSSLSSLQEEEIRRCVQIGLLCVHESPEDRPTMTLILSLLGKEVDFIDRPKPPAETEWTGIKGEASTSTAPPIASTMGAR

SEQ ID NO: 6 - protein, 796 aa, Leavenworthia sp.
MTTHNNSYTFFPLFLLVISFILRMSINGFSLTARESVKLSEDTRNIVSPGEIFEMGLFKAATSLTDIDGWYLGIWYKQLPRIVVWIANRDSHLSNSTATLKMSNTNLFLHDDQSGRTVWNTNLINQINEETLVAELLDNGNFVLKYSNGKSSLWQSFDYPTDTLLPGMKLGLDRTKNLNKTLTAWASLYDPSSGSYVFKIENWKVSHGLLYDTGQIDSRTGPSYSNIVNITETEEEISHSLNITTNVGSISLLQMMYTGSLQLLEFIGGERHILFHFPDGTCDFYNTCGYNTYCNTSSNCECIPGFQPGGQYAWGLTKSKPRCVRNLQLSCQEREFKKIRNMKLPDTEYAIVDTKVGLEECEKRCLMNCNCTAFANIDMRNGGSDCVMWTGDLLDMRSYNNTEGQDLYVKLPAEDLGGKKNINTIIGSVIGGLGLFSLLCYWLVITRNRSRSNSQETSQTIEDWGSICMDYDVIATATENFSDSNTLGKGGFGTVYKGQLPDGQYIAVKKMTEMSQGGVEGFANEMKLIARVQHSNLIRLLGFCSTADHRLLVYEYIENSSLDTYIFDTTEQYVLNWEKRFEIIKGIVKGLIYLHQDSRFRIIHLDLKPNNILLDKDMIPKISDFGLAKILEGNATEGRAPTAVGTLGYIDPNYSKHNIYSAKSDVYSFGVLLLEIVSGKRNMDFLNSFDGTSLLTHIWNSWSKGEVLEIVDPVLKIASLTSLQAEEILKCVHIGLLCVHELPEDRPTMSLVGSLLGKEVDFIDRPKPPAEIGSKEAKGEASTVTSPQITFSMDAR

SEQ ID NO: 7 - protein, 808 aa, Leavenworthia sp.
MMTDNNSYTFFPLFLLVVSFVLSKSINGFSLTAREPVKLSEDKRSIISPDEIFELGLFKATRLSGIDGWYLGIWYKRLPATVVWIANRDHPLSNSTATLKISNTNLFLDDEQSGPPVWKTNLINHINEEPLVAELLDNGNFVLRYSNRKSFLWQSFDYPTDTLLPGMKLGWDRTKNLNKTLTSWASLYDPSSGNYDFEIENWKVSHGLITNTGQPEFRTDLSYSNLVNIAETEEEISHSLNITTNVSSISILQLTSAGALRLTELIGGEKRTLFYFPNDRCDYYNSCGENSYCNTSNNCECMAGFQWGGQYAWGLTKSRQICVRKSQLSCHEKEFKKIRNMKLPDTEYAIVDIKVGLEECEKRCLKNCNCTAFAITDMGNGGSGCVMWTGDLIDLRSYMTEGQDLYVKLPADDLGGKRNINIKPIIGSIIGGLGLLGLVILCYWLVITRNRSKSNSPSITEPPSSDSSKVFEDWGSICMDYDVIATATENFSDSNTLGKGGHGTVYKGQLPDGHKIAVKKMTADSKGGLTGLGNEINLIAKVQHSNLIRLLGSCSTSRPDHNLLVYEYVENSSLDTYIFDTTGQYVLNWEKRFEIIKGIVRGLIYLHQDSRFRIIHLDLKPNNILLDKKMIPKISDFGLAQTLEGNATIGFARTAVGTFGYIAPELRNDNVYSVKSDVYSFGVMLLEIVSGKKNMEFIKNFDGTSLLRIIWDSWSKGEVLEIVDPVLKDSSLSSLQEEEIRRCVQIGLLCVHESPEDRPTMTLVLSLLGKEVDFIDRPKPPAETEWTGIKGEASTSTAPPIGSTMGAR

SEQ ID NO: 8 - protein, 769 aa, Capsella rubella
MNLGEDKGTIVSPGKIFELGLFKGTTSVPYIDRWYLGIWYKRFPEAVVWVANRDNHLHNSTATLIFSSSSSTLKLVDQSGGVVWTSQLRNRINNQRIVPELLDNGNFVFREQLAAGFLWESFDSPTDTLLPGMKLGWDRRTNVNTTSLRSWKSLYDPSYGDYKFQVEIWELSQGFIWKNEDMYLQSRIGLSNHDRIFNITESSEEATCTLAMSSNASLHSALRMTFTGSLQLFVERNLVWSIPFDQCDVYDACGFNSYCAISSSKLHCICLPGFHQLPDSKTGCVRRSQLSCPERVDFTLMNNMKLPSTEGTIVDSRAGIEECRARCATNCTCTAFASTDIRNGESGCVMWNGDLVDVRSESSNRGQDLYVKHAVPDLGLNRKTIIGSTVGGCLLLVLSIIILCFWLKKKNLAKLNYLGNVSKETNQDLIIKRDEDWGSKVIDFEVIAAATNNFSDNNKLGKGGFGIVYKGQLSHGQEIAVKRLSEMTPKGVEGFAIETKLIAMLQHTNLVRLVGFCSNADEKILVYEFLENSSLDRYLFDTTRGSLLNWEYRMKITLGIVRGLVYLHHDSRFRIIHLDLKPANVLLDKDMSPKISDFGMAKILGGDETEAHVTTVKGTFGYIAPEYRNDGTISVKSDVFSFGVMVLEIVSGKRNIDFLNLDDGSTLLSYIWQRWSEGNGLEIVDPAIKDSSSSVFPQVLRCIQIGLLCVQPLPEDRPTMSAVGLMLAREAEVIPLPRSPVEIGSSSRGGQEESVSGTVPDITMFIECR

SEQ ID NO: 9 - protein, 782 aa, Brassica napus
MFRRLSFLVFIFTSVLSFEVWFSENERIVSPSSIFELGLFKDRTGWYLGIWFRQFPGRVVWTGNRGSPLYSSEGKLQISSSAGIQLFDESGYMTWHRDLTSPAAEDDAPLSAYLSDTGNFIVSNYSGGILWGSFDYPSNVLIPGMVLGYYPGLDYIRTITYDDIFHEGGTETGYEHYIWGSSGTKICRIDPIYTTKAMIQTRTTNSYTYSLRRNTTTSYYASLKMSDTGFLIWSEWTRRDRKLKDLVIAPSDICDKYTTCGSGTNTYCSMNPLKSCECFPGFRPQTDSERNQDSYALHGHCVRKSPLACSDDDGFQLLKNMKLPETDNWTISYEGVGLEECKERCLTTCNCTAFANTDMPTGVRSCVMWTVSLEDTRRNRGQNLYVKLAALDMGSNQNKKKRIIGFTVGAIVLLLLIVVVTFCCCWKRYNRTEVDTVTPTENVSRSAPEEETTGSLTSLFMEFDVIAQATNNFSDEIGSGGFAKVYKGRLLDGRDIAVKRLYKLTTHAIQGFWNEVNLIAVLQHTNLVRLIGFVDDPDTKILVYEYLPRSSLNTYIYDTTRSDVLDWNKRMDIAKGIARGLLYLHQDSRVRIIHLDLKLSNVLLCDQMIPRISDFGTAKRLDGEDTEVVASSATGTYGYMAPEYAIDGVCSVKADVFSFGVLLLEMVSGINAREFYWKNDYKSFVGFMWNLWLQGKVLDIVDPYFTSSSSSSSSYQPEEALRCIQIGLLCVQAHREDRPPMASIILMLGSQNELISLPKPPADLLLLQDPQGESFTASVATG

SEQ ID NO: 10 - protein, 838 aa, Arabidopsis lyrata
MRGVIPKYHQSHNFFFFVFVVSTLFLPALSIYANTLLSTESLTIASNQTIVSLGDDFELGFFKPAASLRDGDRWYLGIWYKTISIRTYVWVANRDHPLYSSAGTLKISGINLVLLNQSNIAVWSTNLTGAVRSPPVAELLPNGNFVLRYSKTNGQDILLWQSFDYPTDTLLPHMKLGLDLKTGNNRLLTSWKNSFDPSSGYISYKLETLGLPEFFMWRNEVPIFRSGPWDGTRLSGIPEMQRWKDINISYNFTENKEEVAFTFRVTTPNVYSRLIMNSEGFLQLSRWNPTLSEWNVFWRSSTSDCNGYQSCTPYSYCDTNTTPNCNCIKGFAPQNPQEGALDNTNTECVRKTQLSCDGDGFFWLRNMKPPDTSGAIVDKRIGLKECEERCIKECNCTAFSNMNIQDGGKGCVIWTKELADIRRYADGGQDLYVRLAAVDLVTEKANNNSGKTRTIIGLSVGAIALIFLSFTIFFLWRRHKKAREIAQYTECGQRVGRQNLLETDEDDLKLPLMEYDVVAMATDDFAITNKLGEGGFGTVYKGRLIDGEEIAVKKLSDVSTQGTNEFRTEMILIAKLQHINLVRLLGCFADADDKILVYEYLENLSLDYYIFDETKSSDLNWQTRFNIINGIARGLLYLHKDSRCKVIHRDLKTSNILLDKDMIPKISDFGLARIFARDEEEATTRRIVGTYGYMAPEYAMDGVYSEKSDVFSFGVVILEIVTGKKNRGFTSSDLDTNLLSYVWRNMEEGTGYKLLDPNMMDSSSQAFKLDEILRCITIGLTCVQEYAEDRPMMSWVVSMLGSNTDIPKPKPPGYCLAISSDPWTSTTIEYTTTEVEPR

SEQ ID NO: 11 - protein, 845 aa, Arabidopsis lyrata
MRGVRSIYHHSITLCFFAVLVVLILFCCAFSIHANTLSSTESLTISRNLTIVSPGKIFELGFFKPSTRPRWYLGIWYKKIPERTYVWVANRDTPLSNSVGTLKISDGNLVILDHSNIPIWSTNTKGDVRSPIVAELLDTGNLVIRYFNNNSQEFLWQSFDFPTDTLLPEMKLGWDRKTGLNRFLRSYKSSNDPTSGSFSYKLETGVYSEFFMLAKNSPVYRTGPWNGIQFIGMPEMRKSDYVIYNFTENNEEVSLTFLMTSQNTYSRLKLSDKGEFERFTWIPTSSQWSLSWSSPKDQCDVYDLCGPYSYCDINTSPICHCIQGFEPKFPEWKLIDVAGGCVRRTPLNCGKDRFLPLKQMKLPDTKTVIVDRKIGMKDCKKRCLNDCNCTAYANTDIGGTGCVMWIGELLDIRNYAVGSQDLYVRLAASELGKEKNINGKIIGLIVGVSVVLFLSFITFCFWKWKQKQARASAAPNVNPERSPDILMDGMVIPSDIHLSTENITDDLLLPSTDFEVIVRATNNFSVSNKLGEGGFGIVYKGRLHNGKEFAVKRLSDLSHQGSDEFKTEVKVISRLQHINLVRILGCCASGKEKMLIYEYLENSSLDRHLFDKTRSSNLNWQRRFDITNGIARGILYLHHDSRCRIIHRDLKASNILLDKNMIPKISDFGMARIFSDDVNEAITRRIVGTYGYMSPEYAMDGIYSEKSDVFSFGVMLLEIVTGMKNRGFFNSDLDSNLLSYVWRNMEEEKGLAVADPNIIDSSSLSPTFRPDEVLRCIKIALLCVQEYAEDRPTMLSVVSMLGSETAEIPKAKAPGYCVGRSLHDTDFSSSLTWTFGFAFSDIEPR

SEQ ID NO: 12 - protein, 850 aa, Arabidopsis lyrata
MRGEVPNKHHSYTFFFVLFFALVLFPDFSISANTLSATESLTISSNKTIVSPGGVFELGFFKILGDSWYLGIWYKNVSEKTYVWVANRDKPLSNSIGILKITNANLVLLNHYDTPVWSTNLTGAVRSPVVAELHDNGNFVLRDSKTNASDRFLWQSFDFPTNTLLPQMKLGWDHKRGLNRFLTCWKNSFDPSSGDYMFRLDTQGLPEFFGLKNFLEVYRTGPWDGHRFSGIPEMQQWDDIVYNFTENSEEVAYTFRLTDQTLYSRFTINSVGQLERFTWSPTQQEWNMFWSMPHEECDVYGTCGPYAYCDMSKSPACNCIKGFQPLNQQEWESGDESGRCRRKTRLNCRGDGFFKLMNMKLPDTTAAMVDKRIGLKECEKKCKNDCNCTAYASILNGGRGCVIWIGEFRDIRKYAAAGQDLYIRLAAADIRERRNISGKIIILIVGISLMLVMSFIMYCFWKRKHKRTRARATASTIERIQGFLTNGYQVVSRRRQLFEENKIEDLELPLTEFEAVVIATGNFSESNILGRGGFGMVYKGRLPDGQDTAVKRLSEVSAQGTTEFMNEVRLIARLQHINLVRLLSCCIYADEKILIYEYLENGSLDSHLFKINQSSKLNWQKRFNIINGIARGLLYLHQDSRFKIIHRDLKASNVLLDKNMTPKISDFGMARIFERDETEANTRKVVGTYGYMSPEYAMDGIFSVKSDVFSFGVLVLEIISGKRNRGFYNSNQDNNLLSYTWDNWKEGEGLKIVDPIIIDSSSSFSMFRPYEVLRCIQIGLLCVQERAEDRPKMSSVVLMLGSEKGDIPQPKPPGYCVGRSSLETDSSSSTQRGDESLTVNQITLSVINGR

SEQ ID NO: 13 - protein, 839 aa, Arabidopsis halleri
MRGAVPNYHHFHNFFFFLFVVSVLFCPAFSIFANTLSSTESLTIASNQTIVSLGDDFELGFFRPAASLREGDRWYLGIWYKTISVRTYVWVANRDHPISSSDGTLKISGINLVLLNQSNITVWSTNLTGAVRSPVVAELLPNGNFVLRNSKTNGHDVFMWQSFDNPTDTLLPHMKLGLDLKTGNNRFLTSWKNAYDPSSGYLSYKLEMQGLPEFLMLRGGGPVFRSGPWDGFRFSGIPEMQNWKFAYIVYNFTENKEDVAFTYRVTTPNFYAKLTMRFEGFLELSTWDPDMLEWNVFWVSSTADCNIYMGCTANSFCDTNTSPNCNCIKGFEPRNPQGGALENRSTECVRKTQLNCNGDGFFWLRNMKLPDTSGAIVDKRIGLKECEERCIENCNCTAFANTNIQNGGSGCVLWTRELADIRRYVDAGQDLYVRLAAVDLVTEKGNNNSRKTRTIIGLSVGATALIVLSFTIFFFWRKHKQARGIALYTECGQTGGRLNLLDTTDDDDLKLPLMEYDVVAMATNDFSISNKLGEGGFGTVYKGRLIDGEEIAVKKLSDVSTQGTNEFRTEMILIAKLQHINLVRLLGCFADEDDKILVYEYLENLSLDYYIFDETKSSELNWQTRFNIINGIARGLLYLHKDSRCKVIHRDLKTSNILLDKDMIPKISDFGLARIFARDEEEATTRRIVGTYGYMAPEYAMDGVYSEKSDVFSFGVVILEIVTGKKNRGFTSSDLDTNLLSYVWRNMEEGTGYKLLDPNMIDSSSQAFKLDEILRCITIGLTCVQEYAEDRPMMSWVVSMLGSDTDIPKPKPPGYCLAISSDPWTSTTIEYTTTEVEPR

SEQ ID NO: 14 - protein, 847 aa, Arabidopsis halleri
MRALPNNHHFYILVIFFLLRSALPINVNTLSSTESLTISSNRTIVSLGDVFELGFFNPTPSSRDGDRWYLGIWYKEIPKRTYVWVANRDNPLSNSTGTLKISDNNLVLVDQFNTLVWSTNVTGAVRSLVVAELLANGNLVLRDSKINETDGFLWQSFDFPTDTLLPEMKLGWDLKTGVNKFLRSWKSPYDPSSGDFSYKLETREFPEFFLSWSNSPVYRSGPWEGFRFSGMPEMQQWTNIISNFTENREEIAYTFRDTDQNIYSRLTMSSSGYLQRFKWISNGEDWNQHWYAPKDRCDMYKKCGPYGICDTNSSPECNCIKGFQPRNLQEWSLRDGSKGCVRKTRLSCSEDAFFWLKNMKLPDTTTAIVDRRLGVKECREKCLNDCNCTAFANADIRGSGCVIWTGDLVDIRSYPNGGQDLCVRLAAAELEERNIRGKIIGLCVGISLILFLSFCMICFWKRKQKRLIALAAPIVYHERNAELLMNGMVISSRRRLSGENITEDLELPLVELDAVVMATENFSNANKVGQGGFGIVYKGRLLDGQEIAVKRLSKTSLQGTNEFKNEVRLIAKLQHINLVRLLGCCVEVDEKMLIYEYLENLSLDSYIFDKNRSWKLNWQMRFNITNGIARGLLYLHQDSRCRIIHRDLKASNVLLDKDMTPKISDFGMARIFGREETEANTKKVVGTYGYMSPEYAMDGVFSMKSDVFSFGVLLLEIISGKRNKGFYNSDNDLNLLGCVWRNWTEGKGLEIVDPIILESSSSTVILQEILKCMQIGLLCVQERAEDRPRMSSVVAMLGSETAVVPQPKLPGYCVGRSPLETDSSRSKQHDDESWTVNEITLSVIDAR

SEQ ID NO: 15 - protein, 853 aa, Arabidopsis halleri
MRGFRNIYRHSHTFSFLLVFFVLILFFPAFSSTVNTLSATESLTISSNRTIVSPNDVFELGFFKPGTSSRWYLGIWYKTILQRTYVWVANRDKPLINPIGTLKISNTNLVLLDSSDTLVWSTNLTERDVISPVVAQLLDNGNFVLRYSNKDVQSEFLWQSFHFPTDTLLPQMKIGLDRKTEFNRFLRSWRSADDPASGDYSFKLKTRGVPEFFIWVKQNTRMYRSGPWNGIRFSGMPEMLEFDYMVYNFTENREEIVYTFLMTNHSIYSRLTMTPAGYLQQSTWFPTEEEASWVSPNEQCDTYRICGPYGYCDMITSPICNCIKGFTPRYSEAWKLKDGASGCVRKTPVSCNGKDEFVQLKNMKLPDTTSAVVDKRIGLNECRERCLNDCNCTAFANINIQNRGSGCVVWTRELLDIRNYPAAGQDLYVKIAAADYGDERNQRGKIIGLTVGVSLMVLLSFIIFCLWKRKQMLARATATPTVLQERNQDLLMIGVVISSRRHLSEENITEDLELPSMELKAVVMATENFSDCNKLGQGGFGIVYKGRLLDGQEIAVKRLSETSDQGVHEFKNELRLIARLQHINLVRLLGCCVDEGEKMLIYEYMENLSLDSHLFDKTRSCKLNWQMRFDITTGIARGILYLHQDSRCRIIHRDLKASNVLLDKDMTPKISDFGMARIFGREETEANTRKVVGTYGYMSPEYAMDGIFSMKSDVFSFGVLLLEIISGKRNKGFYNSNGDLNLLGFVWRNWKEGKWTEIIDPAIIDSSSSSLSDFQPQEVLRCIQVGLLCVQERAEERPTMSSVVVMLGSETAAIPHPKPPGYCVGRNLLETVSSSSDESCTVNQITISIMDAR

SEQ ID NO: 16 - protein, 849 aa, Brassica sp.
MKGVRNIYHHSYSSFLLVFVVTILFHPALSIYINTLSSTESLTISSNRTLVSPGDVFELGFFETNSRWYLGMWYKKLPYRTYIWVANRDNPLSNSTGTLKISGSNLVILGHSNKSVWSTNLTRGNERSPVVAELLANGNFVMRDSNNNDASKFSWQSFDYPTDTLLPEMKLGYNLKKGLNRFLVSWRSSDDPSSGDYSYKLEPRRLPEFYLLQGDVREHRSGPWNGIRFSGILEDQKLSYMVYNFTENSEEVAYTFRMTNNSFYSRLTLSSTGYFERLTWAPSSVIWNVFWSSPANPQCDMYRMCGPYSYCDVNTSPSCNCIQGFDPRNLQQWALRISLRGCKRRTLLSCNGDGFTRMKNMKLPETTMAIVDRSIGLKECEKRCLSDCNCTAFANADIRNGGTGCVIWTGNLADMRNYVADGQDLYVRLAVADLVKKSNANGKIISLIVGVSVLLLLIMFCLWKRKQNREKSSAASIANRQRNQNLPMNGIVLSSKRQLSGENKIEELELPLIELEAIVKATENFSNSNKIGQGGFGIVYKGILLDGQEIAVKRLSKTSVQGVDEFMNEVTLIARLQHVNLVQILGCCIDADEKMLIYEYLENLSLDSYLFGKTRRSKLNWKERFDITNGVARGLLYLHQDSRFRIIHRDLKVSNILLDRNMVPKISDFGMARIFARDETEANTMKVVGTYGYMSPEYAMGGIFSEKSDVFSFGVMVLEIITGKRNRGFYEDNLLSYAWRNWKGGRALEIVDPVIVNSFSPLSSTFQLQEVLKCIQIGLLCVQELAENRPTMSSVVWMLGNEATEIPQPKSPGCVKRSPYELDPSSSRQRDDDESWTVNQYTCSVIDAR

SEQ ID NO: 17 - protein, 855 aa, Brassica sp.
MKGVQNSYTFCFLLVFVVLILVHPALSIYFNILSSTESLTISGNRTLVSPGDVFELGFFRTTSSSRWYLGIWYKKVYFRTYVWVANRDNPLSRSIGTLRISNMNLVLLDHSNKSVWSTNLTRGNERSPVVAELLANGNFVMRDSNNNDASGFLWQSFDFPTDTLLPEMKLGYDLKTGLNRFLTAWRNSDDPSSGDYSYKLENRELPEFYLLKSGFQVHRSGPWNGVRFSGIPENQKLSYMVYNFTENSEEVAYTFRMTNNSFYSRLKVSSDGYLQRLTLIPISIAWNLFWSSPVDIRCDMFRVCGPYAYCDGNTSPLCNCIQGFDPWNLQQWDIGEPAGGCVRRTLLSCSDDGFTKMKKMKLPDTRLAIVDRSIGLKECEKRCLSDCNCTAFANADIRNGGTGCVIWTGHLQDIRTYYDEGQDLYVRLAADDLVKKKNANWKIISLIVGVSVVLLLLLLIGFCLWKRKQNRAKAMATSIVNQQRNQNVLMNTMTQSDKRQLSRENKADEFELPLIELEAVVKATENFSNCNELGRGGFGIVYKGMLDGQEVAVKRLSKTSLQGIDEFMNEVRLIARLQHINLVRILGCCIEADEKILIYEYLENSSLDYFLFGKKRSSNLNWKDRFAITNGVARGLLYLHQDSRFRIIHRDLKPGNILLDKYMIPKISDFGMARIFARDETQVRTDNAVGTYGYMSPEYAMYGVISEKTDVFSFGVIVLEIVIGKRNRGFYQVNPENNLPSYAWTHWAEGRALEIVDPVILDSLSSLPSTFKPKEVLKCIQIGLLCIQERAEHRPTMSSVVWMLGSEATEIPQPKPPVYCLIASYYANNPSSSRQFDDDESWTVNKYTCSVIDAR

SEQ ID NO: 18 - protein, 859 aa, Brassica sp.
MKGVHNIYHHSYTFSFLLVFLALILFHPALSTYVNTMSSSESLTISSNRTLVSPGGVFELGFFKPSGRSRWYLGIWYKKVSQKTYAWVANRDNPLSNSIGTLKISGNNLVLLGQSNNTVWSTNLTRENVRSPVIAELLPNGNFVMRYSNNKDSSGFLWQSFDFPTDTLLPEMKLGYDFKTGRNRFLTSWRSYDDPSSGKFTYELDIQTGLPEFILINRFLNQRVVMQRSGPWNGIEFSGIPEVQGLNYMVYNYTENSEEIAYSFQMTNQSIYSRLTVSDYTLNRFTRIPPSWGWSLFWSLPTDVCDSLYFCGSYSYCDLNTSPYCNCIRGFVPKNRQRWDLRDGSHGCVRTTQMSCSGDGFLRLNNMNLPDTKTASVDRTIDVKKCEEKCLSDCNCTSFATADVRNGGLGCVFWTGDLVEIRKQAVVGQDLYVRLNAADLDFSSGEKRDRTGTIIGWSIGVSVMLILSVIVFCFWRRRQKQAKADATPIVGNQVLMNEVVLPRKKIHFSGEDEVENLELSLMEFEAVVTATEHFSDFNKVGKGGFGVVYKGRLVDGQEIAVKRLSEMSAQGTDEFMNEVRLIAKLQHNNLVRLLGCCVYEGEKILIYEYLENLSLDSHLFDETRSCMLNWQMRFDIINGIARGLLYLHQDSRFRIIHRDLKASNVLLDKDMTPKISDFGMARIFGQDETEADTRKVVGTYGYMSPEYAMNGTFSMKSDVFSFGVLLLEIISGKRNKGFCDSDSNLNLLGCVWRNWKEGQGLEIVDRVIIDSSSPTFRPREILRCLQIGLLCVQERVEDRPMMSSVVLMLGSETALIPQPKQPGYCVSQSSLETYSSWSKLRDDENWTVNQITMSIIDAR

SEQ ID NO: 19 - protein, 850 aa, Arabidopsis thaliana
MRGLPNFYHSYTFFFFFLLILFPAYSISANTLSASESLTISSNNTIVSPGNVFELGFFKPGLDSRWYLGIWYKAISKRTYVWVANRDTPLSSSIGTLKISDSNLVVLDQSDTPVWSTNLTGGDVRSPLVAELLDNGNFVLRDSKNSAPDGVLWQSFDFPTDTLLPEMKLGWDAKTGFNRFIRSWKSPDDPSSGDFSFKLETEGFPEIFLWNRESRMYRSGPWNGIRFSGVPEMQPFEYMVFNFTTSKEEVTYSFRITKSDVYSRLSISSSGLLQRFTWIETAQNWNQFWYAPKDQCDEYKECGVYGYCDSNTSPVCNCIKGFKPRNPQVWGLRDGSDGCVRKTLLSCGGGDGFVRLKKMKLPDTTTASVDRGIGVKECEQKCLRDCNCTAFANTDIRGSGSGCVTWTGELFDIRNYAKGGQDLYVRLAATDLEDKRNRSAKIIGSSIGVSVLLLLSFIIFFLWKRKQKRSILIETPIVDHQLRSRDLLMNEVVISSRRHISRENNTDDLELPLMEFEEVAMATNNFSNANKLGQGGFGIVYKGKLLDGQEMAVKRLSKTSVQGTDEFKNEVKLIARLQHINLVRLLACCVDAGEKMLIYEYLENLSLDSHLFDKSRNSKLNWQMRFDIINGIARGLLYLHQDSRFRIIHRDLKASNILLDKYMTPKISDFGMARIFGRDETEANTRKVVGTYGYMSPEYAMDGIFSMKSDVFSFGVLLLEIISSKRNKGFYNSDRDLNLLGCVWRNWKEGKGLEIIDPIITDSSSTFRQHEILRCIQIGLLCVQERAEDRPTMSLVILMLGSESTTIPQPKAPGYCLERSLLDTDSSSSKQRDDESWTVNQITVSVLDAR

SEQ ID NO: 20 - protein, 843 aa, Arabidopsis thaliana
MRSVPNYHHSFFIFLILILFLAFSVSPNTLSATESLTISSNKTIISPSQIFELGFFNPASSSRWYLGIWYKIIPIRTYVWVANRDNPLSSSNGTLKISGNNLVIFDQSDRPVWSTNITGGDVRSPVAAELLDNGNFLLRDSNNRLLWQSFDFPTDTLLAEMKLGWDQKTGFNRILRSWKTTDDPSSGEFSTKLETSEFPEFYICSKESILYRSGPWNGMRFSSVPGTIQVDYMVYNFTASKEEVTYSYRINKTNLYSRLYLNSAGLLQRLTWFETTQSWKQLWYSPKDLCDNYKVCGNFGYCDSNSLPNCYCIKGFKPVNEQAWDLRDGSAGCMRKTRLSCDGRDGFTRLKRMKLPDTTATIVDREIGLKVCKERCLEDCNCTAFANADIRNGGSGCVIWTREILDMRNYAKGGQDLYVRLAAAELEDKRIKNEKIIGSSIGVSILLLLSFVIFHFWKRKQKRSITIQTPNVDQVRSQDSLINDVVVSRRGYTSKEKKSEYLELPLLELEALATATNNFSNDNKLGQGGFGIVYKGRLLDGKEIAVKRLSKMSSQGTDEFMNEVRLIAKLQHINLVRLLGCCVDKGEKMLIYEYLENLSLDSHLFDQTRSSNLNWQKRFDIINGIARGLLYLHQDSRCRIIHRDLKASNVLLDKNMTPKISDFGMARIFGREETEANTRRVVGTYGYMSPEYAMDGIFSMKSDVFSFGVLLLEIISGKRNKGFYNSNRDLNLLGFVWRHWKEGNELEIVDPINIDSLSSKFPTHEILRCIQIGLLCVQERAEDRPVMSSVMVMLGSETTAIPQPKRPGFCIGRSPLEADSSSSTQRDDECTVNQITLSVIDAR

SEQ ID NO: 21 - DNA, 1558 bp, Leavenworthia sp.
tgttatgatgctcggaaacgaaacaacaaagattcctcagcctaaaccgccaggttattgcgtgggaagaagtcctcatgacactgattcatcatcgagtaagccacgttatgatgaaccttggacagtgaaccaaatcactatctcggtccttgacgctaggtaatgtgagtacttctccatagcaaatattagatggttcaaaattgtcttaaattaaacttgtattatgtaattgttttattgagacaacttgtaatagatctgcaacaacaatgattggattgatatgtgatttttcagaagattctcagacaacatattgttataaattatggctataagaagacacgaacaaaacttttgtaggttagataattaaattatttaatacttattaatataaaaacaatttattttagtcattataaattagttatatatggtattataaacaaacatggtttatatatattaagtgacaatatctaaaattgttggggatatctaaaagtgttgtatacttaataatcttccaaatttggatttaacattattgtagtcttacctaagtttgaaccaaacaaaaacaaaagtctatgctttaatggaagacgtgaatcatgtgatgtgcctaaattcttgaccaaaaaaaaaaagtcaacgaaatattcagtgaaaaaaaaattaggggttgttattgaatttagatttattatggttttaatgtatttaatgttaagatgtattttgaaatactatctaacaagttggtattttgattacacatgtttaaaaaaaaaatacattcaaatccaaatctaaataaaccaaatcaacattgaataatacttattttaatgtattttaaaatcaacaaataaatatctaattcaataacagtgtattttattttacttttttaaatacataattaaataaattttattgtattttaaaaatacaaaattatccattaaaatacaaaattaaataatcccatcttaaagtttctagaaaaggtgttatctttgtacccaaaataccctcgaccgtacgtcacgaaacaaaagggctaatttcgtcatctcaaacaaaagccattgctgcaaaaaagcttcacggtctacgaaccacctctatctctctctttttatgttttatctgctctgttcttacagggaatcttataaacccttgtctctctgtctctttgagttctaatcttatctgtttctcatttgacactttttaaaagtttcaaagtaggagatggcctttgagttaccaaacgatttcagatgccctatctctcttgagattatgtctgaccctgttattatccaatcgggtcatactttcgatcgggtctctatccaacggtggattgactccggtaatcgaacttgcccaatcaccaagctccctttatcagaaaacccttctttaatccctaatcatgctctccgcagtttaatttctaattttgctcatgtaagccctaaggaaacacttcccaggactcaccaagaacactctcagtctctatcccaagctt

SEQ ID NO: 22 - DNA, 1457 bp, Leavenworthia sp.
  UNSURE (606)..(650): n = A, T, C or G
tgttatgatgctcggaaacgaaacaacaaagattcctcagcctaaaccgccaggttattgcgtgggaagaagtcctcatgacactgattcatcatcgagtaagccacgttatgatgaaccttggacagtgaaccaaatcactatctcggtccttgacgctaggtaatgtgatatgactttatcgagcggtttatgtaattgttttattgagacaacttgtaatagtcctgcaacaacaatgattggattgatatgtgatttttcagaagattctcagacaacatattgttataaattatggctgtaagaagtcgcgaacaaaataggttagataattaaattatttaatacttattaatacaaaaacaatttattttagtcattataaattagttatatatggtattataaacaaacatggtttatatatattaagtgacaatatctaaaattgttggggatatctaaaagtgttgtatacttaataatcttccaaatttggatttaacattattgtagtcttacctaagtttgaaccaaacaaaaacaaaagtctctgctttaatgaaagacgtgaatcatgtgatgtgcctaaattcttgaccnnnnnnnnnnnnnnnnnnnnnnnnnnnnnnnnnnnnnnnnnnnnngttgttattgaatttagatttattatagttttaatgtatttaattttaacatgtattttgaaatactatctaacaagttggtattttgattacacatgttttaaaaaaaaatacattcaaatccaaatctaaataaaccaaatcaacattaaataatacttattttaatgtattttaaaatcaacaaataaatatctaattcaataacaatgtattttattttacttttttaaatacataattaaataatacatgatttcattgtattttaaaaatacaaaattatccattaaaatacaaaattaaataatcccatcttaaagtttctagaaaaggtgttatctttgtacccaaaataccctcgaccgtacgtcacgaaacaaaagggctaatttcgtcatatcaaacaaaagccattgctgcaaaaaagcttcacggtctacgaaccacctctatctctctctttttatgttttatctgctctgttcttacagggaatcttataaacccttgtctctctgtctctttgagttctaatcttatctgtttctcatttgacaccttttaaaagtttcaaagtaggagatggcctttgagttaccaaacgatttcagatgccctatctctcttgagattatgtctgaccctgttattatccaatcgggtcatactttcgatcgggtttctatccaacggtggattgactccggtaatcgaacttgcccaatcaccaagctccctttatcagaaaacccttctttaatccctaatcatgctctccgcagcttaatttctaatttgctcatg

SEQ ID NO: 23 - protein, 546 aa, Leavenworthia sp.
KLGLDRTKNLNKTLTAWASLYDPSSGSYVFKIENWKVSHGLLYDTGQIDSRTGPSYSNIVNITETEEEISHSLNITTNVGSISLLQMMYTGSLQLLEFIGGERHILFHFPDGTCDFYNTCGYNTYCNTSSNCECIPGFQPGGQYAWGLTKSKPRCVRNLQLSCQEREFKKIRNMKLPDTEYAIVDTKVGLEECEKRCLMNCNCTAFANIDMRNGGSDCVMWTGDLLDMRSYNNTEGQDLYVKLPAEDLGGKKNINTIIGSVIGGLGLFSLLCYWLVITRNRSRSNSQETSQTIEDWGSICMDYDVIATATENFSDSNTLGKGGFGTVYKGQLPDGQYIAVKKMTEMSQGGVEGFANEMKLIARVQHSNLIRLLGFCSTADHRLLVYEYIENSSLDTYIFDTTEQYVLNWEKRFEIIKGIVKGLIYLHQDSRFRIIHLDLKPNNILLDKDMIPKISDFGLAKILEGNATEGHAPTAVGTLGYIDPNYSKHNIYSAKSDVYSFGVLLLEIVSGKRNMDFLNSFDGTSLLTHIWNSWSKGEVLEIVDPV

SEQ ID NO: 24 - DNA, 20 bp, Artificial Sequence
  Oligonucleotide
acctttggtggcagagcttc

SEQ ID NO: 25 - DNA, 21 bp, Artificial Sequence
  Oligonucleotide
aatgctgtacagttgcaattc

SEQ ID NO: 26 - DNA, 19 bp, Artificial Sequence
  Oligonucleotide
ttctatggcagagctttga

SEQ ID NO: 27 - DNA, 20 bp, Artificial Sequence
  Oligonucleotide
acytcttctcrcattcttcc

SEQ ID NO: 28 - DNA, 20 bp, Artificial Sequence
  Oligonucleotide
aagttacaacaccgatgagg

SEQ ID NO: 29 - DNA, 20 bp, Artificial Sequence
  Oligonucleotide
agtacaggatctactatctc

SEQ ID NO: 30 - DNA, 20 bp, Artificial Sequence
  Oligonucleotide
accaagattctcggtttagg

SEQ ID NO: 31 - DNA, 20 bp, Artificial Sequence
  Oligonucleotide
agtacaggatctactatctc

SEQ ID NO: 32 - DNA, 24 bp, Artificial Sequence
  Oligonucleotide
gctgatggcgatgaatgaacactg

SEQ ID NO: 33 - DNA, 20 bp, Artificial Sequence
  Oligonucleotide
agcacgaaattgccgttatc

SEQ ID NO: 34 - DNA, 35 bp, Artificial Sequence
  Oligonucleotide
cgcggatccgaacactgcgtttgctggctttgatg

SEQ ID NO: 35 - DNA, 20 bp, Artificial Sequence
  Oligonucleotide
aattgccgttatccagaagc

SEQ ID NO: 36 - DNA, 20 bp, Artificial Sequence
  Oligonucleotide
ttgaaattgtcagtggcaag

SEQ ID NO: 37 - DNA, 23 bp, Artificial Sequence
  Oligonucleotide
gcgagcacagaattaatacgact

SEQ ID NO: 38 - DNA, 20 bp, Artificial Sequence
  Oligonucleotide
agatagtagatcctgtactc

SEQ ID NO: 39 - DNA, 32 bp, Artificial Sequence
  Oligonucleotide
cgcggatccgaattaatacgactcactatagg

SEQ ID NO: 40 - DNA, 20 bp, Artificial Sequence
  Oligonucleotide
aatggccaaaagtgtatggc

SEQ ID NO: 41 - DNA, 20 bp, Artificial Sequence
  Oligonucleotide
ggaaacatgagatgagcaac

SEQ ID NO: 42 - DNA, 19 bp, Artificial Sequence
  Oligonucleotide
atggctaaaagtgtaaggc

SEQ ID NO: 43 - DNA, 20 bp, Artificial Sequence
  Oligonucleotide
ttatagagcaccaacaaagg

SEQ ID NO: 44 - DNA, 23 bp, Artificial Sequence
  Oligonucleotide
aacaggtaagtcttgttaacttc

SEQ ID NO: 45 - DNA, 22 bp, Artificial Sequence
  Oligonucleotide
ttccaacaatttactctaaagc

SEQ ID NO: 46 - DNA, 20 bp, Artificial Sequence
  Oligonucleotide
ggaaacatgagatgagcaac

SEQ ID NO: 47 - DNA, 20 bp, Artificial Sequence
  Oligonucleotide
aacaaggccttactctgcag

SEQ ID NO: 48 - DNA, 20 bp, Artificial Sequence
  Oligonucleotide
tggcttactagtttcatcag

SEQ ID NO: 49 - DNA, 20 bp, Artificial Sequence
  Oligonucleotide
tttccttgtggggaactttc

SEQ ID NO: 50 - DNA, 20 bp, Artificial Sequence
  Oligonucleotide
gcttcatcatctatctaacg

SEQ ID NO: 51 - DNA, 20 bp, Artificial Sequence
  Oligonucleotide
ctccaaagatctcggatttc

SEQ ID NO: 52 - DNA, 20 bp, Artificial Sequence
  Oligonucleotide
cgttaacagagtagcagcaa

SEQ ID NO: 53 - DNA, 20 bp, Artificial Sequence
  Oligonucleotide
tggtctcttgtgtgttcaag

SEQ ID NO: 54 - DNA, 20 bp, Artificial Sequence
  Oligonucleotide
aagcttgggatagagactga

SEQ ID NO: 55 - DNA, 20 bp, Artificial Sequence
  Oligonucleotide
tatgcacttccacatgctat

SEQ ID NO: 56 - DNA, 21 bp, Artificial Sequence
  Oligonucleotide
ctttgcgatccacatctgctg

SEQ ID NO: 57 - DNA, 21 bp, Artificial Sequence
  Oligonucleotide
ttgtgttgacatggttgcagg

SEQ ID NO: 58 - DNA, 20 bp, Artificial Sequence
  Oligonucleotide
ttgtgttgttattaagaggg

SEQ ID NO: 59 - DNA, 18 bp, Artificial Sequence
  Oligonucleotide
ttctatggcagagctttg

SEQ ID NO: 60 - DNA, 20 bp, Artificial Sequence
  Oligonucleotide
tagcttcttcatcactttgg

SEQ ID NO: 61 - DNA, 20 bp, Artificial Sequence
  Oligonucleotide
tatcttcctttcggagtagc

SEQ ID NO: 62 - DNA, 20 bp, Artificial Sequence
  Oligonucleotide
ttccagccttgacacgtatc

SEQ ID NO: 63 - DNA, 20 bp, Artificial Sequence
  Oligonucleotide
taagccgatctgtacgcatc

SEQ ID NO: 64 - DNA, 20 bp, Artificial Sequence
  Oligonucleotide
ttcttcaaacctgcaacgag

SEQ ID NO: 65 - DNA, 20 bp, Artificial Sequence
  Oligonucleotide
acaagtaacaaacagcctcc

SEQ ID NO: 66 - protein, 811 aa, Artificial Sequence
  Consensus amino acid sequence of Figure S1
MXTXNNSYTFFXLFLLXXSFXXXXSINGFSLTAREXVKLSEXXRXIXSPXEIFEXGLFKXATXXXXXDGWYLGIWYKXLPXXVVWIANRDXXLSNSTATLKXSNXNLFLXDXQSGXXVWXTNXINXINXEEXXVAELLDNGNFVLXYSNXKSXXWQSFDYPTDXLLPGMKLGXDRTKNLNKTLTXWASLXXPXSGXYXFXIENWXVSHGLXXXXGQXXXRTXXXYXNXVNIXETEXEISHSLNITXNVXSXSXLQXXXXGXLXLXEXIGGEXXXLFXFPXXXCDXYNXCGXNXYCXTXXXCXCXXGFQXGGQYAXGLTKSXXXCXRXXXLSCXEXEFKKIRNXKLPDTZYAIXDXKVGLEECEKRCLXNCNCTAFAXXDMXNGXSXCVMWTGDLXDXRSYXXXEGQDLYVKLPAXDLXGKXNXNXXXIIGSXIGGLGLXXLXXXCYWLVITRNRSXXNSXXXXXXXXXXXSXXXEDWGSICMDYDVIATATXNFSDSNTLGKGGXGTVYKGQLPDGXXIAVKKMTXXSXGGXXGXXNEXXLIAXVQHSNLIRLLGXCSTXXXDHXLLVYEYXENSSLDTYIFDTTXQYXLBWEXRFEIIKGIVXGLIYLHQDSRFRIIHLDLKPNNILLDKXMIPKISDFGLAXXLEXNATXGXXXTAVGTXGYIXPXXXXXNXYSXKSDVYSFGVXLLEIVSGKXNMXXXXXFDGTSLLXXIWBSWSKGXVLEIVDPVLKXXSLXSLQXEEIXXCVXIGLLCVHEXPEDRPTMXLXXSLLGKEVDFIDRPKPPAEXXXXXXKGEASTXTXPXIXXXMXAR

SEQ ID NO: 67 - protein, 93 aa, Arabidopsis lyrata
MAKIVKLVSFFITLVVMIFLLISTGIAKIEGKRPHLCNPTRMKAPPGTCNVQNGNKLCRKLCMGPVDNGYFRGFEFGYCRATPKGRYCECSNC

SEQ ID NO: 68 - protein, 807 aa, Arabidopsis lyrata
MTMTRSVPHGNHFYTSFFFFVFQLVVLIPSIASYDSTFSPTRPLRITENETIVSPEGIFELGFFKPATRFQERDRWYLGIWYKRFTTRVVWVANRDDPLSSSIGTLKVDNSNIILLDQSGGVAWTTSLTKNMINNQLLVAKLLDNGNFVLRFSNSSSYLWQSFDFPTDTLLPGMKLGWDRRTNHTKSLISWNSSDDPSSGRYVYKIDTLKPSQGLIIFGDDLPVSRPGPSYRKLFNITETDNEITHSLGISTENVSLLTLSFLGSLELMAWTGEWNVVWHFPRNLCDSYGACGQNSYCNIVNEKTKCNCIQGFQGDQQHAWDLLDSEKRCLRKTQLSCDSKAEFKQLKKMDFPDTKTSIVDTTVGSEECRKSCLTNCNCTAFANTEWGCVRWTSDLIDLRSYNTEGVDLYIKLATADLGVNKKTIIGSIVGGCLLLVLSFIILCLWIRRKKRARAIAAANVSQERNRDLTINTTEDWGSKHMDFDVISTATNHFSELNKLGKGGFGIVYKGRLCDGQEIAVKRLSKMSPIGVEGFTVEAKLIALVQHVNVIRLIGFCSNADEKILVYEFLENSSLDTYLFDSTRGSVLNWDTRFDIAKGIIRGLVYLHQDSRFRIIHLDLKPSNILLGKDMVPKISDFGMARILGGDETEAHVTTVTGTFGYIAPEYRSDGVLSVKSDVFSFGVMLLEIISGKRNIDFLHLNDGSTLLSYMWNHWSQGNGLEIVDPAIKDSSSSSQQILRCVQIGLMCVQELPEDRPTMSSVGLMLGRETEAIPQPKSPVETGSSSGGQQESESGTVPEITLFIEGR

SEQ ID NO: 69 - protein, 903 aa, Artificial Sequence
  Consensus amino acid sequence Figure 2S(A)
MXXXXXXXXXXXXXXXXXXXXXXXXXXXXXXXXXXXXXXXXXXXXXXXXXXXXXSXXXXFEXGXFXXXXXXXXXXXWYLGXWXXXXXXXXXXWXXNRXXXXXXXXXXLXXXXXXXXXXXXXXXXXXXXWXXXXXXXXXXXXXXXXXXLXXXGNXXXXXXXXXXXXXXXWXSFXXPXBXLXXXMXXGXXXXXXXXXXXXXXXXXXXXXXXXXXXXXXXXXXXXXXXXXXXXXXXXXXXXXRXXXXXXXXXXXXXXXXXXXXXXXXXXXXXXXXXXXXXXXXXXXXXXXXXXXXXXXXXXXXXXXXXXXXXXXXXXXXXXXXXXCBXXXXCXXXXXXXCXXXXXXXXCXCXXGFXXXXXXXXXXXXXXXXXXXXXCXRXXXXXCXXXXXFXXXXXXXXPXTXXXXXXXXXXXXXXCXXXCXXXCXCTXXXXXXXXXXXXXCVXWXXXXXXXRXXXXXXXXBLXXXXXXXXXXXXXXXXXXXXXXXIIXXXXGXXXXXXXXXXXXXXXXWXXXXXXXXXXXXXXXXXXXXXXXXXXXXXXXXXXXXXXXXXXXXXXXXXXXXXXXXXXXXATXXFXXXXXXGXGGXXXVYKGXXXXGXXXAVKXXXXXXXXXXXXXXXEXXXIXXXQHXNXXXXXXXXXXXXXXXXXLXYEXXXXXSLBXXXXXXXXXXXLBWXXRXXIXXGXXXGXXYLHXDSRXXXIHXDLKXXNXLLXXXMXPXISDFGXAXXXXXXXXXXXXXXXXGTXGYXXPXXXXXXXXSXKXDVXSFGVXXLEXXXXXXXXXXXXXXXXXXXXXXXWXXXXXXXXXXXXDXXXXXXXXXXXXXXXXXZXXXCXXXXLXCXXXXXEXRPXMXXXXXXLXXXXXXXXXXXXPXXXXXXXXXXXXXXXXXXXXXXXXXTXXXXXXXXXXXR

SEQ ID NO: 70 - DNA, 1503 bp, Artificial Sequence
  Consensus nucleotide sequence shown on Figure S4
tgttatgatgctcggaaacgaaacaacaaagattcctcagcctaaaccgccaggttattgcgtgggaagaagtcctcatgacactgattcatcatcgagntaagccacgnttatgatgaaccttggacagtgaaccaaatcactatctcggtccttgacgctaggtaatgtgantaykwctynnnnnnnnntatyrrryggtnnnnnnnnnnnnnnnnnnnnnnnnnnnttatgtaattgttttattgagacaacttgtaatagwyctgcaacaacaatgattggattgatatgtgatttttcagaagattctcagacaacatattgttataaattatggctrtaagaagwcrcgaacaaaannnnnntaggttagataattaaattatttaatacttattaatayaaaaacaatttattttagtcattataaattagttatatatggtattataaacaaacatggtttatatatattaagtgacaatatctaaaattgttggggatatctaaaagtgttgtatacttaataatcttccaaatttggatttaacattattgtagtcttacctaagtttgaaccaaacaaaaacaaaagtctmtgctttaatgraagacgtgaatcatgtgatgtgcctaaattcttgaccnnnnnnnnnnnnnnnnnnnnnnnnnnnnnnnnnnnnnnnnnnnnngttgttattgaatttagatttattatrgttttaatgtatttaatkttaasatgtattttgaaatactatctaacaagttggtattttgattacacatgtttwaaaaaaaaatacattcaaatccaaatctaaataaaccaaatcaacattraataatacttattttaatgtattttaaaatcaacaaataaatatctaattcaataacartgtattttattttacttttttaaatacataattaaataannnnnnatttyattgtattttaaaaatacaaaattatccattaaaatacaaaattaaataatcccatcttaaagtttctagaaaaggtgttatctttgtacccaaaataccctcgaccgtacgtcacgaaacaaaagggctaatttcgtcatmtcaaacaaaagccattgctgcaaaaaagcttcacggtctacgaaccacctctatctctctctttttatgttttatctgctctgttcttacagggaatcttataaacccttgtctctctgtctctttgagttctaatcttatctgtttctcatttgacacyttttaaaagtttcaaagtaggagatggcctttgagttaccaaacgatttcagatgccctatctctcttgagattatgtctgaccctgttattatccaatcgggtcatactttcgatcgggtytctatccaacggtggattgactccggtaatcgaacttgcccaatcaccaagctccctttatcagaaaacccttctttaatccctaatcatgctctccgcagyttaatttctaantttgctcatg

SEQ ID NO: 71 - protein, 796 aa, Artificial Sequence
  Consensus amino acid sequence Figure S7
MTTHNNSYTFFPLFLLVISFILRMSINGFSLTARESVKLSEDTRNIVSPGEIFEMGLFKAATSLTDIDGWYLGIWYKQLPRIVVWIANRDSHLSNSTATLKMSNTNLFLHDDQSGRTVWNTNLINQINEETLVAELLDNGNFVLKYSNGKSSLWQSFDYPTDTLLPGMKLGLDRTKNLNKTLTAWASLYDPSSGSYVFKIENWKVSHGLLYDTGQIDSRTGPSYSNIVNITETEEEISHSLNITTNVGSISLLQMMYTGSLQLLEFIGGERHILFHFPDGTCDFYNTCGYNTYCNTSSNCECIPGFQPGGQYAWGLTKSKPRCVRNLQLSCQEREFKKIRNMKLPDTEYAIVDTKVGLEECEKRCLMNCNCTAFANIDMRNGGSDCVMWTGDLLDMRSYNNTEGQDLYVKLPAEDLGGKKNINTIIGSVIGGLGLFSLLCYWLVITRNRSRSNSQETSQTIEDWGSICMDYDVIATATENFSDSNTLGKGGFGTVYKGQLPDGQYIAVKKMTEMSQGGVEGFANEMKLIARVQHSNLIRLLGFCSTADHRLLVYEYIENSSLDTYIFDTTEQYVLNWEKRFEIIKGIVKGLIYLHQDSRFRIIHLDLKPNNILLDKDMIPKISDFGLAKILEGNATEGXAPTAVGTLGYIDPNYSKHNIYSAKSDVYSFGVLLLEIVSGKRNMDFLNSFDGTSLLTHIWNSWSKGEVLEIVDPVLKIASLTSLQAEEILKCVHIGLLCVHELPEDRPTMSLVGSLLGKEVDFIDRPKPPAEIGSKEAKGEASTVTSPQITFSMDAR

SEQ ID NO: 72 - protein, 107 aa, Artificial Sequence
  Consensus between SCRL a1-1 and a1-2 of Figure 3
MAKSVXLXSFIXYLMIXMLIXXVXPKPHXSXVPXXXXXXXXXXXXXXXSXXXXSTCXXQKXXXXCXXXCXEXXXYXXGKCXSXXXGXXCXCXXXXXCLXXXXMFPFK

SEQ ID NO: 73 - protein, 884 aa, Artificial Sequence
  Consensus amino acid sequence as shown on Figure S2(B)
MTMTRXXXXXNXXXXXFXXXXXXXXXXXXXXXXXXXXXXXXXXXXXXEXXXXIXSPXXIFEXGXFKXXXXXXXXXXWYLGIWXXXXXXXVVWXXNRXXXLXXSXXXLXXXXXXXXXXXXXZSGXXXWXXXXXXXXXXXXXXXXXXLXDXGNFXXXXXXXXXXXWXSFDXPXBXLXPGMXLGXXXXXBXXXXXXXXXXXXXXXXXXXXXXXXXXXXXSXXXXXXXXXXXXXXRXXXXXXXXXXXXXXXXXXXXXXLXXXXXXXXXXXLXXXXXGXLXXXXXXXXXXXXXXXXXXPXXXCDXYXXCGXXXNXYCXXXXXXXXCXCXXGFXXXXXXXXXXXXXXXXXXCXRXXXLXCXXXXXFXXXXXXXXPXTXXXXIXXXXXGXEECXXXCXXXCXCTAFAXXXXXXXXXXCVXWXXXLXDXRXXXXXXGXBLYXKXXXXDXXXXXXXXXXXIIGXXXGXXXLXXLXXXXXXXCXWXXXXXXXXXXXXXXXXXXXXXXXXXXXXXXXXXXXXSXXXXXXVIXXATXXFSXXXXXGXGGXXXVYKGXLXXGXXIAVKXXXXXXXXXXXGXXXEXXLIAXXQHXNXXRLXGXXXXXXXDXXXLVYEXXXXSSLBXYXXDXTXXXXXXXXXXXXXXXXXXXXXXYXXXXXXXXSRXXXXXXXXXXXXXXXXXXXXXXXXDXGXXXXXXXXXXXXXXXXXXXXXXXXXPXXXXXXXXSXXXXXXXXXXXXXXXXXXXXXXXXXXXXXXXXXXXXXXXXWXXXXXXXXXXXXXXXXXXXXXXXXXXXXXXXXXXXXLXXXXXXXDRXXXXXXXXXXXXXXXXXXXXXXXXXXXXXXXXXXXXXXXXXXXXXXXXXXXXXXXXXXXXXXXXXXXEXXXXXXXXXXXXXXXX

SEQ ID NO: 74 - DNA, 30 bp, Artificial Sequence
  Oligonucleotide
cccgggatgacgactctcaacaattcttac

SEQ ID NO: 75 - DNA, 27 bp, Artificial Sequence
  Oligonucleotide
gcggccgctcatcgagcgcccatggtg

SEQ ID NO: 76 - DNA, 30 bp, Artificial Sequence
  Oligonucleotide
ctgaccgcggatgttgaacatgttctgatg

SEQ ID NO: 77 - DNA, 24 bp, Artificial Sequence
  Oligonucleotide
gaattgcaactgtacagcatttgc

SEQ ID NO: 78 - DNA, 30 bp, Artificial Sequence
  Oligonucleotide
ctgacccgggatggccaaaagtgtatggct

SEQ ID NO: 79 - DNA, 33 bp, Artificial Sequence
  Oligonucleotide
ctgagcggccgcttatttaaatggaaacatgag

SEQ ID NO: 80 - DNA, 31 bp, Artificial Sequence
  Oligonucleotide
agtcaagcttagtcttcttgtacacgtcgac

SEQ ID NO: 81 - DNA, 32 bp, Artificial Sequence
  Oligonucleotide
ctgacccgggggcttagtttaatgaacacatg

SEQ ID NO: 82 - DNA, 30 bp, Artificial Sequence
  Oligonucleotide
ctgaccgcggtaaccatggccatgaattgc

SEQ ID NO: 83 - DNA, 30 bp, Artificial Sequence
  Oligonucleotide
ctgacccgggtatctccttccaaatagttc

SEQ ID NO: 84 - DNA, 30 bp, Artificial Sequence
  Oligonucleotide
ctgacccgggatgacgactcacaacaattc

SEQ ID NO: 85 - DNA, 30 bp, Artificial Sequence
  Oligonucleotide
tgagcggccgctcaacgagcatccatggag

SEQ ID NO: 86 - DNA, 30 bp, Artificial Sequence; identical to SEQ ID NO: 78
  Oligonucleotide

SEQ ID NO: 87 - DNA, 33 bp, Artificial Sequence; identical to SEQ ID NO: 79
  Oligonucleotide

(19) United States
(12) Patent Application Publication
(10) Pub. No.: US 2015/0322445 A1
(43) Pub. Date: Nov. 12, 2015

(54) SELF-INCOMPATIBILITY SYSTEM FOR MAKING BRASSICACEAE HYBRID

(71) Applicant: THE ROYAL INSTITUTION FOR THE ADVANCEMENT OF LEARNING/MCGILL UNIVERSITY, Montreal (CA)

(72) Inventors: Daniel J. Schoen, St. Lambert (CA); Sier-Ching Chantha, Montreal (CA)

(21) Appl. No.: 14/705,015

(22) Filed: May 6, 2015

Related U.S. Application Data
(60) Provisional application No. 61/989,035, filed on May 6, 2014.

(51) Int. Cl. C12N 15/82; C07K 14/415; A01H 5/10

(52) U.S. Cl. CPC C12N 15/8231; A01H 5/10; C07K 14/415

(57) ABSTRACT

The present disclosure provides a genetic system based on the co-expression of the Lal2 polypeptide and the SCRL polypeptide for conferring self-incompatibility to otherwise self-compatible Brassicaceae plants. The genetic system is especially useful for generating Brassicaceae hybrids.

### CROSS-REFERENCE TO RELATED APPLICATIONS AND DOCUMENTS

This application claims priority from U.S. provisional patent application 61/989,035 filed on May 6, 2014 which is incorporated herewith in its entirety. This application also contains an electronic version of the sequence listing (USPTOSequencelistingasfiled.txt of 221 Ko) which is incorporated herewith in its entirety.

### TECHNOLOGICAL FIELD

The present disclosure relates to a sporophytic self-incompatibility system for making a transgenic plant of the Brassicaceae family (such as Camelina) suitable for hybridization.

### BACKGROUND

The excessive use of petroleum-derived products has brought about both market-based and environmental concerns, and has spurred interest in the development of alternative sources of oil. One promising alternative is plant-derived oil. One of the possible crops that could serve as a substitute for industrial grade oil is the plant species Camelina sativa, an annual plant of the Brassicaceae family. Apart from its value as a replacement for petroleum in some industrial applications, the oil of this species has numerous desirable nutritional qualities, such as high levels of omega-3 fatty acids, polyunsaturated fats, long-chain fatty acids, vitamin E and antioxidants. Among its advantages to growers are the considerable yields achievable with low levels of input, its adaptability to a wide range of growing conditions (including to lands not normally used to grow other crops), and its seeds that contain a large amount of oil. At present the number of elite varieties of Camelina sativa is quite small, and additional gains in developing this crop into an even more viable oil substitute will benefit from advanced plant breeding.

Plant breeding typically involves both selection and controlled cross-pollination. For example, crosses are made between different lines, and desirable characteristics are retained by selection of progeny. Following such selective improvement, the bulk production of commercially useful quantities of seed may require cross-pollination, typically on a scale of thousands of plants or more. For instance, synthetic varieties are created by crossing a number of genotypes that possess good combining ability, whereas hybrids are typically created by cross pollination of two (or sometimes three or four) different parental lines. Hybrid and synthetic varieties offer a number of advantages such as precise genotype identification and multiplication, facilitation of combining multiple traits into one variety, profitable seed sales on an annual basis that attracts capital and provides incentives for continuous crop improvement, as well as the prospect of hybrid vigor (or heterosis)—the phenomenon of increased growth in the offspring over the parents achieved in a hybrid cross.

Because Camelina sativa is a naturally self-fertilizing plant (flowers are normally spontaneously self-pollinated), hybridization can only be achieved if self-pollination is prevented. For small numbers of crosses, removal or destruction of anthers (the pollen-bearing organs) prior to spontaneous self-pollination is possible, but it is not practical for large-scale breeding and seed production. To circumvent this problem in other naturally-self-pollinating crops, breeders have relied upon two types of interventions: (1) cytoplasmic male sterility (CMS), in which one parent in the cross possesses a genetic mutation that prevents the production of fertile pollen; and/or (2) self-incompatibility (SI), in which plants identify and reject their own pollen, and thus only produce seed with the pollen of another genotype. A CMS system has been prophecized for Camelina sativa based on one that exists for Canola (refer to, for example, WO2011/034945 filed on Sep. 15, 2010). Unfortunately, CMS and SI are not present in Camelina sativa populations, and thus there currently exists no simple means by which hybrid and synthetic lines can be produced.

Self-incompatibility (SI) is a widespread plant reproductive system that prevents inbreeding by facilitating the rejection of self-pollen. It is a major evolutionary feature of the flowering plants. SI is a complex phenotype whose functioning requires co-evolution among several interacting components. It has been proposed that SI evolved several times in the angiosperms, a hypothesis supported by molecular investigations that have also helped pinpoint the genes that control pollen specificity, pollen recognition, and the downstream reactions that mediate cessation of pollen tube growth. The evolutionary loss of SI leading to self-compatibility (SC) and the potential for the shift to self-fertilization is often stated to be irreversible.

Despite increasing knowledge of the mechanisms that underlie SI, the question remains as to how such a complex system could have evolved independently in many different angiosperm lineages. One answer may lie in the phenomenon of neo-functionalization of genes. It has been noted that the mechanisms that underlie SI share a number of features with another important plant function, namely pathogen recognition and rejection. Moreover, it has become increasingly clear that evolution can reshuffle and reshape functions through exon recruitment and domain swapping and so it is conceivable that SI could have evolved by co-opting genes with receptor and signaling roles that initially functioned in plant defense. Neo-functionalization of genes has been shown to be most likely when there are strong selection pressures. The avoidance of inbreeding and its negative fitness consequences provide one such selective context.

In the sporophytic type of self-incompatibility (SSI), the pollen and stigma SI phenotypes (or “specificities”) are controlled by the diploid genotype of the parent (the sporophyte). SSI is known from 10 families of flowering plants. It has been best characterized in the Brassicaceae family. In Arabidopsis and Brassica (and several other closely related Brassicaceae), the self-incompatibility locus (S locus) contains two tightly linked genes that have been shown to be principally responsible for the SI phenotype. One of these genes, the S-locus receptor kinase (SRK), produces a transmembrane receptor expressed in the stigma. The extracellular domain of this protein can bind to the secreted protein ligand produced by the other S-locus gene, the S-locus cysteine-rich gene (SCR, also known as SP11), which is expressed in the tapetum of anthers, coating pollen with the protein product. When self-pollen recognition occurs, it initiates a signaling cascade that prevents self-pollen hydration and growth of the pollen tube.

It would be highly desirable to be provided with a genetic system to limit self-fertilization in Brassicaceae plants, such as Camelina, in order to develop hybrids of such plants. In some embodiment, the genetic system is a temporal one and can allow reversal to self-fertilization when necessary. Preferably, the genetic system does not exhibit consequences on overall fitness of the plant comprising such genetic system.

### BRIEF SUMMARY

The present disclosure provides a genetic system for introducing sporophytic self-incompatibility in Brassicaceae plants, including Camelina plants. The genetic system comprises two components: a transgene coding for a Lal2 polypeptide and a transgene coding for a SCRL polypeptide. Plants possessing both transgenes exhibit self-incompatibility and can be used for producing hybrids.

According to a first aspect, the present disclosure provides a first isolated nucleic acid molecule encoding for a Lal2 polypeptide, wherein the Lal2 polypeptide is capable of intracellular signaling upon specifically binding to a SCRL polypeptide. The Lal2 polypeptide is at least one of: a polypeptide having the amino acid sequence of SEQ ID NO: 66, a polypeptide encoded by a Lal2 gene ortholog, and a variant polypeptide of the polypeptide of (i) or (ii). In an embodiment, the SCRL polypeptide is derived from a SCRL gene that is located within 10,000 base pairs from a corresponding Lal2 gene. In an embodiment, the first isolated nucleic acid molecule is a complementary DNA (cDNA). In another embodiment, the Lal2 polypeptide has at least one cysteine residue at positions corresponding to amino acid residues 283, 289, 295, 301, 303, 324, 332, 362, 366, 370, 372 or 387 of SEQ ID NO: 66. In still another embodiment, the Lal2 polypeptide has the amino acid sequence of any one of SEQ ID NO: 5 to 7.

According to a second aspect, the present disclosure provides a first vector comprising a promoter operatively linked to a first transgene encoding a transgenic Lal2 polypeptide, wherein the first transgene comprises the first isolated nucleic acid molecule described herein. In an embodiment, the promoter is a stigma-specific or a stigma-active promoter.

According to a third aspect, the present disclosure provides a first Agrobacterium host cell comprising the first vector described herein.

According to a fourth aspect, the present disclosure provides a first transgenic Brassicaceae plant or cell comprising the first vector described herein. In an embodiment, the first transgenic Brassicaceae plant or cell is hemizygous or homozygous for the first transgene. In yet another embodiment, the first transgenic Brassicaceae plant or cell is obtained by transforming a Brassicaceae cell with the first Agrobacterium host cell described herein. In yet another embodiment, the first transgenic Brassicaceae plant has a stigma expressing the transgenic Lal2 polypeptide encoded by the first isolated nucleic acid molecule. In still a further embodiment, the first transgenic Brassicaceae plant or cell is a Camelina plant or cell.

According to a fifth aspect, the present disclosure provides a second isolated nucleic acid molecule encoding for a SCRL polypeptide, wherein the SCRL polypeptide is capable of specifically binding to a Lal2 polypeptide so as to allow the Lal2 polypeptide to mediate intracellular signaling. The SCRL polypeptide is at least one of a polypeptide having the amino acid sequence of SEQ ID NO: 72; a polypeptide encoded by a SCRL gene ortholog; and a variant polypeptide of the SCRL polypeptide described herein. The SCRL polypeptide is derived from a SCRL gene located within 10,000 bp of a corresponding Lal2 gene. In an embodiment, the second isolated nucleic acid is a complementary DNA (cDNA). In another embodiment, the SCRL polypeptide has at least one cysteine residue residues at positions corresponding to amino acid residues 56, 65, 69, 80, 89, 91, and 97 of SEQ ID NO: 72. In still another embodiment, the SCRL polypeptide comprises the amino acid sequence of any one of SEQ ID NO: 1 to 2.

According to a seventh aspect, the present disclosure provides a second vector comprising a promoter operatively linked to a second transgene encoding a transgenic SCRL polypeptide, wherein the second transgene comprises the second isolated nucleic acid molecule described herein. In an embodiment, the promoter is an anther tapetum-specific or an anther tapetum-active promoter.

According to an eighth aspect, the present disclosure provides a second Agrobacterium host cell comprising the second vector described herein.

According to a ninth aspect, the present disclosure provides a second transgenic Brassicaceae plant or cell comprising the second vector described herein. In an embodiment, the second transgenic Brassicaceae plant or cell is hemizygous or homozygous for the second transgene. In yet another embodiment, the second transgenic Brassicaceae plant or cell is obtained by transforming a Brassicaceae cell with the second Agrobacterium host cell described herein. In still a further embodiment, the second transgenic Brassicaceae plant described herein has an anther expressing the second transgene encoded by the second isolated nucleic acid. In yet a further embodiment, the second transgenic Brassicaceae plant or cell is a Camelina plant or cell.

According to a tenth aspect, the present disclosure provides a method for producing a self-incompatible transgenic Brassicaceae plant, said method comprising (a) crossing the first transgenic Brassicaceae plant described herein with the second transgenic Brassicaceae plant described herein so as to obtain a crossed transgenic Brassicaceae and (b) identifying the crossed transgenic Brassicaceae plant as being self-incompatible if the crossed Brassicaceae plant is a double-transgenic for the first transgene and the second transgene.

According to an eleventh aspect, the present disclosure provides a self-incompatible transgenic Brassicaceae plant or cell having (i) a first transgene comprising the first isolated nucleic acid molecule described herein, (ii) a second transgene comprising the second isolated nucleic acid molecule described herein and (iii) being a double-transgenic for the first transgene and the second transgene. In an embodiment, the self-incompatible transgenic Brassicaceae plant or cell is obtained by the method described herein. In yet another embodiment, the self-incompatible transgenic Brassicaceae plant or cell is a Camelina plant or cell.

According to a twelfth aspect, the present disclosure provides a genetic system for producing a self-incompatible Brassicaceae plant. The genetic system comprises (i) at least one of the first isolated nucleic acid described herein, the first vector described herein, the first transgenic Agrobacterium host cell described herein and the first transgenic Brassicaceae plant or cell described herein and (ii) at least one of the second isolated nucleic acid described herein, the second vector described herein, the second transgenic Agrobacterium host cell described and the second transgenic Brassicaceae plant or cell described herein.

According to a thirteenth aspect, the present disclosure provides a method for producing a hybrid Brassicaceae plant or cell. The method comprises (a) crossing the self-incompatible transgenic Brassicaceae plant described herein with a second Brassicaceae plant so as to provide a crossed Brassicaceae plant and (b) identifying the crossed Brassicaceae plant as an hybrid Brassicaceae if the crossed Brassicaceae exhibits a first trait unique to the self-incompatible transgenic Brassicaceae plant and a first trait unique to the second Brassicaceae plant. In an embodiment, the method further comprises providing self-compatibility to the identified hybrid Brassicaceae.

According to an fourteenth aspect, the present disclosure provides a hybrid Brassicaceae plant or cell hemizygous or homozygous for the first transgenic nucleic acid molecule as defined herein and for the second transgenic nucleic acid molecule defined herein. In an embodiment, the hybrid Brassicaceae plant or cell is produced by the method described herein. In yet another embodiment, the hybrid Brassicaceae plant or cell is a Camelina plant or cell.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 provides a schematic representation of aligned sequences and protein domain organization of Lal2 alleles and closely related gene family members. The amino acid sequences of Leavenworthia a1-1, a2 and a4 LaLal2 alleles, Arabidopsis lyrata AlLal2 (NCBI Gene ID 930517), A. lyrata SRK14 (a class B SRK allele), Brassica oleracea SRK12, Arabidopsis halleri SRK43, as well as A. thaliana ARK3 and ARK1 were aligned along with their annotated domains. Thick black bars represent amino acid regions and thin lines represent gaps of one or more amino acids introduced to optimize the alignment. Arrows highlight alignment gaps observed specifically in all Lal2 sequences. Circles indicate alignment gap found in region of all Lal2 sequences and in AlSRK14 corresponding to the DUF3660 and DUF3403 domains of all other sequences. Protein domains are represented with patterned boxes and their accession numbers are indicated in parentheses next to corresponding names in the legend.

FIG. 2A-B provides a phylogenetic reconstruction of the relationships among Lal2, ARK and SRK sequences and among Lal2-like sequences in the Brassicaceae. Bayesian 50% consensus phylogeny for the full coding sequence of Lal2, ARK and SRK sequences. (A) Posterior probabilities for each bifurcation are indicated at the nodes. Lal2 sequences form a clade separate and distinct from ARK and SRK sequences (vertical bar). The phylogeny in (B) was generated in PhyML and used to test for codon-specific positive selection with the branch-site model. Positive selection was allowed in the foreground branches (indicated with dashed lines). Outgroups are identified by their NCBI gene ID numbers.

FIG. 3 provides an alignment of amino acid sequences of Leavenworthia and A. lyrata SCRL alleles. The A. lyrata AlSCRL sequence corresponds to NCBI Gene ID_{—}9305018 (SEQ ID NO: 67). The a1-1 and a1-2 LaSCRL alleles (respectively SEQ ID NO: 1 and 2) are from the SI race and have full open reading-frames while the a2 and a4 alleles (respectively SEQ ID NO: 3 and 4) are from SC races and encode truncated proteins. In the a1-1 and a1-2 alleles, gray box highlights the predicted signal peptide; arrow indicates conserved position of the intron; arrowhead marks the predicted cleavage site of the a1-1 and a1-2 preproteins. Cysteines found in the predicted mature protein sequences are boxed. Asterisks represent stop codons. Hyphens represent gaps that were introduced to optimize the alignment. Consensus between Leavenworthia alleles a1-1 and a1-2 LaSCRL is shown in SEQ ID NO: 72.

FIG. 4A-B illustrates the characterization of the S locus genomic region in Leavenworthia. (A) VISTA alignment showing sequence conservation in a selected region of the Leavenworthia a1-1, a2 and a4 S haplotypes. The a4 S haplotype was used as the reference sequence. Arrows indicate genes annotated using the A. thaliana reference genome. (B) Structural gene organization of the Leavenworthia S haplotypes and synteny with a region of A. thaliana chromosome 4. Arrows represent genes in the Leavenworthia S haplotypes (black) and in the syntenic region of A. thaliana (white). Thick gray dashed lines represent unavailable sequences in the a2 and a1-1 S haplotypes. Thin dashed lines indicate orthologous genes within Leavenworthia. For clarity, only syntenic genes were identified above corresponding white arrows in the A. thaliana region and are connected to Leavenworthia orthologous genes by thin gray lines. Vertical arrows indicate the 5′ or 3′ borders of regions syntenic to A. thaliana chromosome 4.

FIG. 5 illustrates synteny of a genomic region in Arabidopsis lyrata scaffold 7 and the Lal2 S-locus region of Leavenworthia. Mauve alignment of A. lyrata scaffold 7 region between positions 852,500 and 1,060,200 (from gene AT4G37830/NCBI gene ID 9303002 to AT4G39950/NCBI gene ID 9302972) and a selected region of the a4 fosmid clone sequence. Collinear and homologous regions are represented by blocks connected by a line. In the Leavenworthia sequence, the block located below the thin black line represents an inverted region. Annotated genes are shown above the A. lyrata panel and below the Leavenworthia panel. Genes were annotated with the A. thaliana reference genome and the NCBI Gene ID numbers for A. lyrata genes is also given. Gray arrows represent genes found in both A. thaliana and Leavenworthia syntenic regions; black arrows represent genes found in A. thaliana only. For clarity, only genes found in the syntenic region of Leavenworthia are identified and also NCBI Gene ID 9302985. Underlined are SCRL and LaLal2 genes in the Leavenworthia core S-locus region and their orthologous A. lyrata genes NCBI gene ID_{—}9305018 (AlSCRL) and NCBI gene ID_{—}9305017 (AlLal2).

FIG. 6A-B illustrates the Arabidopsis S locus in Leavenworthia and S locus positions in Brassicaceae genera. (A) Mauve alignment showing synteny of the A. thaliana chromosome 4 region comprised between positions 11,349,900 bp and 11,492,100 bp (from genes At4g21330 to At4g21620) and a selected region of 64,800 bp of Leavenworthia genome scaffold 1085. Annoted genes are shown above the A. thaliana panel and below the Leavenworthia panel. Black arrows represent genes found in both A. thaliana and Leavenworthia syntenic regions; white arrows represent genes found in A. thaliana only. Boxed area highlights the A. thaliana core S-locus region that corresponds to a large deletion in Leavenworthia. For clarity, only syntenic genes and genes found in A. thaliana core S locus are identified above corresponding arrows. (B) Phylogeny of five Brassicaeae genera for which S locus synteny information is available. Black square denotes that the S locus is found in a region flanked by genes At4g21350 (PUB8) and At4g21380 (ARK3). Black circle denotes that the S locus is found in a region flanked by genes At1g66680 and At1g66690. Black star denotes that the S locus is found in a region flanked by genes At4g37910 and At4g40050.

FIG. 7A-B provides the expression pattern analysis of Lal2 and SCRL by RT-PCR in vegetative and reproductive tissues. (A) Expression of the LaLal2 and LaSCRL in a Leavenworthia plant homozygous at the a1-1 S haplotype. (B) Expression of AlLal2 and AlSCRL in a self-incompatible A. lyrata plant.

FIG. 8A-B provides the expression analysis by RT-PCR of LaLal2 and LaSCRL alleles in Leavenworthia SI and SC plants homozygous at the S locus. (A) Expression analysis of LaLal2 alleles in stigmas collected two days before anthesis. Asterisks indicate bands corresponding to an alternatively spliced form of LaLal2 transcripts. The ACTIN gene was used as an internal control. (B) Expression analysis of LaSCRL alleles in anthers collected two days before anthesis. Because of the high sequence divergence between the different SCRL alleles, primer pairs used for amplification were allele-specific except for the a2 and a1-2 alleles, for which the same primer pair was used. The ACTIN gene was used as an internal control. Genomic DNA extracted from the four haplotypes was used to amplify SCRL with their respective primer pairs to show that all the primer pairs used in PCR reactions amplify SCRL.

FIG. 9 illustrates possible evolutionary scenarios to account for the unique characteristics of the Leavenworthia S locus. Scenario I: Lal2/SCRL pollen protein-receptor function evolves from SRK/SCR paralogs in the Leavenworthia lineage, following the loss of SRK/SCR-based SI in this lineage. Scenario II: Lal2/SCRL pollen protein-receptor function evolves from SRK/SCR paralogs in the Leavenworthia lineage and two separate S loci coexist for a portion of the history of the Leavenworthia lineage, following by eventual loss of SRK/SCR in this lineage.

FIG. 10A-B provides a sequence analysis of LaLal2. (A) Schematic representation of the alignment of the a4 LaLal2 genomic DNA and cDNA sequences. Exons are represented with white boxes and their sizes in bp are indicated in parenthesis. (B) Alignment of predicted amino acid sequences of the a1-1 (SEQ ID NO: 5), a2 (SEQ ID NO: 6) and a4 (SEQ ID NO: 7) alleles of LaLal2. Amino acid sequences were deduced from cDNA sequences. Consensus sequence (SEQ ID NO: 66) is shown above allele sequences with X representing residues not conserved in the three alleles. Sequences of the predicted protein domains determined by the SMART/Pfam programs for the a1-1 LaLal2 allele are highlighted using the pattern code shown below. Black arrows indicate the twelve conserved cysteine residues in the extracellular domain. The kinase domain possesses the eleven kinase subdomains (I to XI) as established by Hanks et al. (1988).

FIG. 11A-B provides (A) the amino acid sequence alignment of Lal2 alleles and related sequences. Leavenworthia LaLal2 alleles (a1-1 as shown in SEQ ID NO: 5, a2 as shown in SEQ ID NO: 6 and a4 as shown in SEQ ID NO: 7), A. lyrata AlLal2 (NCBI Gene ID 930517 as shown in SEQ ID NO: 68), Lal2-like sequences from C. rubella (Carubv10025960m as shown in SEQ ID NO: 8), B. rapa (Bra010990 as shown in SEQ ID NO: 9) and, a selection of full-length coding sequences of SRK alleles from A. lyrata (SRK14 as shown in SEQ ID NO: 10, SRK01 as shown in SEQ ID NO: 11, SRK25 as shown in SEQ ID NO: 12), A. halleri (SRK28 as shown in SEQ ID NO: 13, SRK13 as shown in SEQ ID NO: 14, SRK43 as shown in SEQ ID NO: 15), and Brassica sp. (SRK12 as shown in SEQ ID NO: 16, SRK54 as shown in SEQ ID NO: 17, SRK60 as shown in SEQ ID NO: 18) as well as A. thaliana ARK3 (SEQ ID NO: 19) and ARK1 (SEQ ID NO: 20) were aligned. AlSRK14 and AhSRK28 belong to class B SRK alleles. Consensus sequence (SEQ ID NO: 69) is shown above sequences with X representing residues not conserved. The approximate positions of protein domains are indicated bellow the aligned sequences. Dashes represent gaps introduced to optimize the alignment. Black arrows highlight alignment gaps observed specifically in all Lal2 sequences. Black circles indicate alignment gaps found in the regions of all Lal2 sequences and in class B AlSRK14 and AhSRK28 alleles corresponding to the DUF3660 and DUF3403 domains in all other sequences. This figure also provides (B) the amino acid sequence alignment of Lal2 alleles as well as those encoded by Lal2 orthologs. Leavenworthia LaLal2 alleles (a1-1 as shown in SEQ ID NO: 5, a2 as shown in SEQ ID NO: 6 and a4 as shown in SEQ ID NO: 7), A. lyrata AlLal2 (NCBI Gene ID 930517 as shown in SEQ ID NO: 68), Lal2-like sequences from C. rubella (Carubv10025960m as shown in SEQ ID NO: 8), B. rapa (Bra010990 as shown in SEQ ID NO: 9) Consensus sequence (SEQ ID NO: 73) is shown above sequences with X representing residues not conserved.

FIG. 12A-B provides a phylogenetic reconstruction of the relationships among Lal2, Lal2-like, ARK, and SRK for different portions of the sequence. Bayesian 50% consensus phylogeny for the S-domain (A) and the transmembrane and kinase domains (B) of Lal2, Lal2-like, ARK and SRK sequences. Posterior probabilities for each bifurcation are indicated at the nodes. Lal2 sequences form a clade separate and distinct from ARK and SRK sequences (vertical bars). The outgroup in each tree is identified by its NCBI gene ID number.

FIG. 13 shows sequence alignment of the ARK3-PUB8 intergenic region in Leavenworthia SC a4 and SI a1-1 plants. Highlighted in light gray are the 3′ end of the coding sequence of ARK3 (top, SEQ ID NO: 21) and the 5′ end of the PUB8 (bottom, SEQ ID NO: 22) orthologs. The a4 sequence was extracted from Leavenworthia scaffold 1085 (FIG. 6A). The a1-1 sequences were obtained by PCR amplification using primers anchored in the ARK3 and PUB8 coding sequences, followed by end-sequencing of PCR products (size of about 1.5 kb). Note that the a1-1 end sequences obtained do not overlap and the sequence corresponding to a stretch of 45 nucleotides of the a4 sequence (between positions 650 and 696) remains unknown. Dark gray horizontal bars above aligned sequences indicate identity between sequences. The ARK3-PUB8 intergenic regions covered by the a1-1 sequences are 93% identical between a1-1 and a4. Consensus sequence is provided at SEQ ID NO: 70.

FIG. 14 provides the genomic organization of the S locus in Sisymbrium irio. An SRK gene sequence was identified in a genome region between gene orthologs of A. thaliana PUB8 and ARK3. Genes were annotated using the A. thaliana reference genome.

FIG. 15 shows a SSCP gel for AlLal2 and AlSCRL from 10 individuals from a single A. lyrata population. The observed banding patterns indicate monomorphism for both loci.

FIG. 16 provides the alignment of the a2 full-length (SEQ ID NO: 6) and a1-2 partial (SEQ ID NO: 23) LaLal2 amino acid sequences. The a1-2 amino acid sequence was deduced from cDNA sequence obtained by using primers anchored in exon 1 and exon 7 of the gene (see Table 1 for primers sequences) and corresponds to positions 169 to 714 of the a2 LaLal2 aa sequence. Dark gray horizontal bars above aligned sequences represent identity between sequences. Note that the available amino acid sequence of a1-2 is identical to that of a2 except for one amino acid residue located in the intracellular kinase domain. The predicted transmembrane domain is highlighted with a light gray box to delimit the extracellular domain versus the intracellular domain. Consensus sequence is provided at SEQ ID NO: 71.

FIG. 17A-B illustrates pollen tube growth in a transgenic Camelina line. (A) Incomplete pollen tube growth as observed in an incompatible cross in line 1-15 pistil pollinated with pollen from line 4-21. (B) Abundant pollen tube growth as observed in a compatible cross in line 1-15 pistil pollinated with pollen from line 4-21.

### DETAILED DESCRIPTION

The present disclosure provides a self-incompatibility system that is useful for providing self-incompatible Brassicaceae plants as well as cells derived therefrom. In some embodiments, the genetic system presented herewith is less leaky than existing self-incompatibility loci, does not affect pollen production (attracts pollinators) and/or is not based on a mitochondrial lesion that could affect plant growth (unlike male sterility), and can be applied successfully in plants of the Brassicaceae family. The genetic system described herein was developed based on Leavenworthia's S locus. In the present disclosure, new data on the Leavenworthia S locus gleaned from fosmid cloning, sequencing, expression analysis, comparative genomic, and crossing studies is presented. While sequence characteristics and tissue expression pattern of both the pollen and stigma genes may support the hypothesis that the previously described Lal2 gene forms a portion of the Leavenworthia S locus, comparative synteny studies, along with closer examination of sequence variation at this locus suggest that the Arabidopsis S-locus ortholog was lost in Leavenworthia following the divergence of the group from the common ancestor with other members of the Cardamineae. In addition, phylogenetic analysis of Lal2, SRK, and other gene family members suggest that SI in this genus is based on genes that have diversified separately and are thus likely paralogous to Arabidopsis SRK and SCR. It is also shown that two separate losses of SI in one species of Leavenworthia (L. alabamica) are likely due to independent mutations in the SCR-like gene coding sequence and/or its promoter. Together these results portray SI as a reproductive system that is more evolutionarily plastic than previously believed.

### Lal2 Polypeptides and Associated Tools

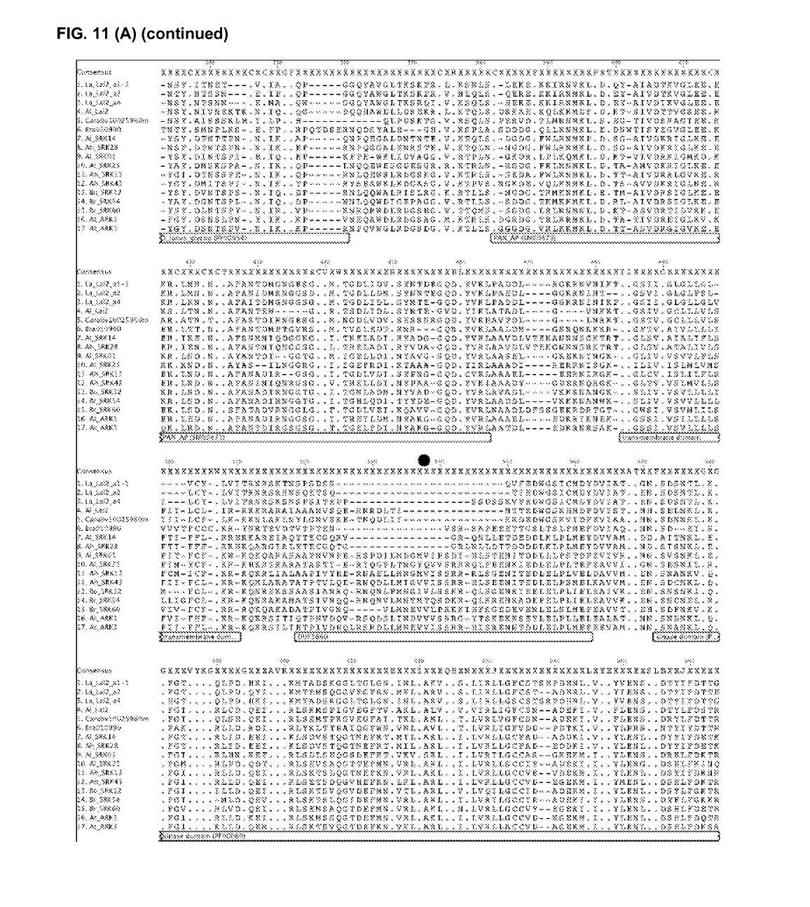

The genetic system described herein comprises, as a first component, a nucleic acid coding for the Lal2 polypeptide. In the context of the present disclosure, a “Lal2 polypeptide” refers to polypeptide encoded by the Lal2 gene. The Lal2 polypeptide is a transmembrane receptor expressed in the stigma of a Brassicaceae plant. Upon specific binding to its cognate ligand (e.g., the SCRL polypeptide), self-recognition occurs and Lal2 is capable of initiating intracellular signaling which will ultimately lead to the prevention of self-pollen hydration and growth of the pollen tube. The cognate ligand of the Lal2 polypeptide is encoded by a gene (e.g., the SCRL gene) that is located at most within 10 000 base pairs of the gene encoding a corresponding Lal2 polypeptide. The Lal2 polypeptide has a signal peptide domain, an extracellular domain responsible for specifically binding to the SCRL polypeptide, a transmembrane domain as well as an intracellular domain that can exhibit kinase activity. As shown on FIG. 10 as well as in the amino acid sequence of SEQ ID NO: 66, the signal peptide domain spans from amino acid residues at positions 1 to 25, the extracellular domain spans from amino acid residues at positions 26 to 426 and the intracellular domain spans from amino acid residues at positions 427 to 811. The kinase domain, located inside the intracellular domain, spans from amino acid residues at positions 494 to 778.

In some embodiments, the Lal2 polypeptide has, consists essentially of or consists of the amino acid consensus sequence of SEQ ID NO: 66. In other embodiments, the Lal2 polypeptide is devoid of a signal peptide and has, consists essentially of or consists of the amino acid sequence located between residues 26 to 811 of SEQ ID NO: 66. Alternatively or in combination, the Lal2 polypeptide can have, consist essentially of or consist of a polypeptide having the residues important for recognizing and binding to the SCRL polypeptide. For example, the Lal2 polypeptide can have, consist essentially of or consist of a polypeptide having at least one, and in some embodiments, at least two, three, four, five, six, seven, eight, nine, ten, eleven or twelve of any one of cysteine residues at positions corresponding to amino acid residues 283, 289, 295, 301, 303, 324, 332, 362, 366, 370, 372 and 387 of SEQ ID NO: 66. In yet further embodiments, the Lal2 polypeptide can have, consist essentially of or consist of the amino acid sequence of any one of SEQ ID NO: 5 to 7. In some further embodiment, the Lal2 polypeptide can have, consist essentially of or consist of the amino acid sequence of SEQ ID NO: 5.

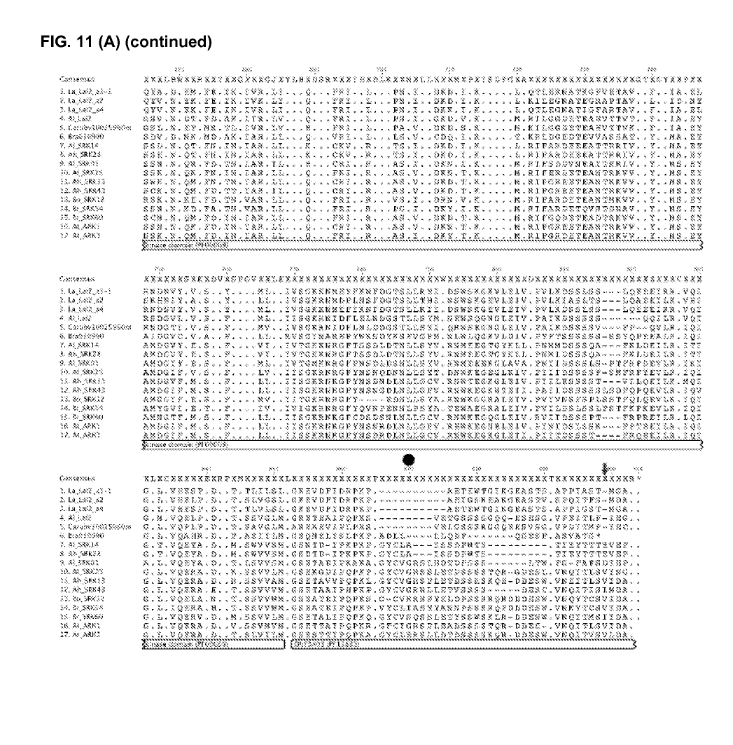

In other embodiments, the Lal2 polypeptide is encoded by an ortholog of the Lal2 gene (e.g., a Lal2 gene ortholog also referred to as a Lal2 ortholog). In the context of the present disclosure, a “Lal2 gene ortholog” is understood to be a gene in a different plant species that evolved from a common ancestral gene by speciation. Still in the context of the present disclosure, a Lal2 gene ortholog encodes a polypeptide have a biological function similar to the Lal2 polypeptide, e.g. it can act as a transmembrane signaling protein for allowing sporophytic self-incompatibility in Brassicaceae. Lal2 orthologs include, but are not limited to genes encoding the following polypeptides Arabidopsis lyrata ALLal2 (NCBI Gene ID 930517 as shown in SEQ ID NO: 68); Capsella rubella CARUBV10025960M (as shown in SEQ ID NO: 8) and Brassica rapa BRA010990 (as shown in SEQ ID NO: 9). Lal2 orthologs specifically exclude Lal2 paralogs such as, for example, genes encoding the following polypeptides, SRK14 (as shown in SEQ ID NO: 10), SRK01 (as shown in SEQ ID NO: 11), SRK25 (as shown in SEQ ID NO: 12); Arabidopsis halleri SRK28 (as shown in SEQ ID NO: 13), SRK13 (as shown in SEQ ID NO: 14), SRK43 (as shown in SEQ ID NO: 15); Brassica sp. SRK12 (as shown in SEQ ID NO: 16), SRK54 (as shown in SEQ ID NO: 17), SRK60 (as shown in SEQ ID NO: 18); as well as Arabidopsis thaliana ARK3 (as shown in SEQ ID NO: 19) and ARK1 (as shown in SEQ ID NO: 20). In an embodiment, the degree of identity of Lal2 orthologs with respect to the Lal2 polypeptide is at least 37.1%, 45.8%, 46.4% in a MUSCLE (MUltiple Sequence Comparison by Log-Expectation) alignment (when determined on the entire open-reading frame of the Lal2 polypeptide). In another embodiment, the Lal2 ortholog encodes a polypeptide having the amino acid sequence set forth in SEQ ID NO: 73 or as shown on FIG. 11B.

In yet another embodiment, the Lal2 polypeptides described herein also encompass Lal2 polypeptide variants. In the context of the present disclosure, the “Lal2 polypeptide variants” are polypeptides that vary in of at least one amino acid residue when compared to the Lal2 polypeptide. This variation can be the addition of an amino acid residue, the removal of an amino acid residue or the modification in the identity of an amino acid residue when compared to the Lal2 polypeptide. In some embodiments, the Lal2 polypeptide variant is a function-conservative variant in which a change in one or more nucleotides in a given codon position of the Lal2 gene results in a Lal2 polypeptide sequence in which a given amino acid residue in the polypeptide has been replaced by a conservative amino acid substitution. The Lal2 polypeptide variants encode a polypeptide having the same biological function as the Lal2 polypeptide, e.g. it can act as a transmembrane signaling protein for allowing self-incompatibility in Brassicaceae. In a further embodiment, the Lal2 polypeptide variants include allelic variations of the Lal2 polypeptide (such as, for example, a1-1 and a2 Lal2 polypeptides). In an embodiment, the degree of identity between the amino acid sequence of the Lal2 variant and the Lal2 polypeptide is at least 70%, 71.8%, 75%, 76%, 77%, 78%, 79%, 80%, 85%, 90%, 95%, 96%, 97%, 98% or 99% in a MUSCLE (MUltiple Sequence Comparison by Log-Expectation) alignment (when determined on the entire amino acid sequence frame of the Lal2 polypeptide). In an embodiment, the degree of identity between the Lal2 variants is provided in function of the consensus sequence of SEQ ID NO: 66, SEQ ID NO: 73 or the sequence set forth in any one of SEQ ID NO: 5 to 7.

In still another embodiment, the Lal2 polypeptides described herein encompass Lal2 polypeptide fragments. In the context of the present disclosure, the “Lal2 polypeptide fragments” are polypeptides that are at least one amino acid residue shorter than the Lal2 polypeptide. For example, one contemplated Lal2 fragment is devoid of a signal peptide and, in some embodiments, can have, consist essentially of or consists of the amino acid residues 26 to 811 of SEQ ID NO: 66 or 31 to 864 of SEQ ID NO: 73. In some embodiments, the deletion can be located at the NH₂ terminal of the Lal2 polypeptide or the COOH terminal of the Lal2 polypeptide. The deletion can be between contiguous amino acids or can affect different non-contiguous amino acids (at numerous positions on the Lal2 polypeptide). The Lal2 polypeptide fragments have the same biological function as the Lal2 polypeptide, e.g. it can act as a transmembrane signaling protein for allowing self-incompatibility in Brassicaceae. In an embodiment, the total number of amino acids in the Lal2 fragment is decreased by 1%, 2%, 3%, 4%, 5%, 6%, 7%, 8%, 9% or 10% when compared to the total amino acid number of the Lal2 polypeptide.

The first nucleic acid molecule of the genetic system described herein can be derived/isolated from a genomic sequence of the Lal2 gene (or the Lal2 ortholog) or a corresponding transcript of such Lal2 gene or Lal2 ortholog. In some embodiments, the Lal2 gene encodes a protein having the amino acid sequence of SEQ ID NO: 66 or 69, for example the amino acid sequence of any one of SEQ ID NO: 5 to 7. In some embodiments, the first nucleic acid molecule is a complementary DNA of a transcript of the Lal2 gene or Lal2 ortholog. In other embodiments, the first nucleic acid molecule is derived from the amplification of the genomic sequence of the Lal2 gene or Lal2 ortholog or of a transcript (for example a messenger RNA transcript) expressed from the Lal2 gene or Lal2 ortholog. In one embodiment, the oligonucleotides used to amplify the genomic sequence of the Lal2 gene or Lal2 ortholog or of a transcript expressed from the Lal2 gene or Lal2 ortholog can be those set forth in Table 1 (for example, Lal-Sdomain5′-F1 & Lal-Sdomain3′-R; LalGenF & LalRcon; TNC_Lal2_Exon1-F & Lal2_Exon7-R1; Lal2-Exon5-F1 & Lal2_Exon7-R1; Lal2_Sdomain5′-F2 & Lal2-Exon7-R1; Al_Lal2_Exon1_F1 & Al_Lal2_Exon7_R2; Al_Lal2_Exon1_F & Al_Lal2_Exon2_R).

The first nucleic acid molecule of the genetic system can be included in a vector intended to be used to produce a transgenic Brassicaceae plant. For example, the first nucleic acid molecule can be used as a transgene and included in an appropriate vector. In some embodiments, the vector can also comprise a promoter operatively linked to the transgene encoding a transgenic Lal2 polypeptide. The promoter can be constitutive or regulated. In some embodiments, the promoter can allow for the expression of the transgene more preferably (and in some embodiments exclusively) in female organs of a Brassicaceae plant such as a Camelina plant. The promoter of the first nucleic acid molecule can be a stigma and/or a style-specific promoter. Alternatively, the promoter of the first nucleic acid molecule can be active (e.g., drive the expression of downstream nucleic acid molecules) in the stigma and/or the style of a plant. Embodiments of such stigma/style-specific and -active promoters include, but are not limited to Nicotiana tabacum promoters of the STMG-type genes (as described in Example 2 below), corresponding STMG-type promoters in other plants which can be isolated/identified STMG-type genes as a probe, promoters isolated from self-incompatibility genes (such as an S-gene, for example as isolated from Nicotiana alata (McClure et al. (1989) Nature 342, 955-957)), female organ-specific promoters identified using other female organ-specific cDNAs, such as cDNA clone pMON9608 (Gasser et al. (1989) Plant Cell 1, 15)) that hybridizes exclusively with a gene expressed only in the ovules of tomato plants, the STIG1 promoter (Goldman M H et al., EMBO Journal 1994), the STG08 promoter, the STG4B12 promoter, the PSTMG07 promoter, the PSTMG08 promoter, the PSTMG4B12 promoter, the PSTMG3C9 promoter, the SLR1 stigma-specific promoter (Hackett R M et al., Plant physiology 1996) and the Lal2 native promoter. In additional embodiments, when the Lal2 polypeptide does not have a signal peptide, the vector can also include, upstream of the Lal2 transgene, and operatively linked to the Lal2 transgene, a nucleic acid molecule encoding a signal peptide that will direct the expression of the transgenic Lal2 polypeptide at the cytoplasmic surface of the plant cell. Embodiments of such signal peptide include, but are not limited to, the signal peptide of amino acid residues 1 to 26 of SEQ ID NO: 66 and of amino acid residues 1 to 30 of SEQ ID NO: 73. In further embodiments, the vector can also comprise a selection marker or a plurality of selection markers that can allow for the identification of cells (e.g., Agrobacterium cells or plant cells) comprising the vector. In yet another embodiment, the vector can be designed to be partly integratable/integrated into the genome of a recipient cell (such as a Brassicaceae cell). For example, the vector can be designed to be integrated into an Agrobacterium cell as well as partly integretable/integrated in the genome of a plant cell (such as a Brassicaceae cell). In such embodiment, the vector that is to be introduced into the Agrobacterium cell comprises an Agrobacterium selection marker and the part of the vector that is to be integrated in the plant cell comprises a plant selection marker. In additional embodiments, the vector can be designed to be able to replicate independently in a non-plant host cell, such as an Agrobacterium host cell.

In still another embodiment, the first nucleic acid molecule or the first vector can be operably linked to the second nucleic acid molecule (encoding the SCRL polypeptide or a variant thereof, described below) or the second vector (comprising the second nucleic acid molecule). In some embodiments, the first and the second nucleic acid molecule may be included in a single vector that may be suitable for expansion in Agrobacterium and, in yet other embodiments, for integration in the Brassicaceae plant or cell.

Although other plant transformation techniques are contemplated, the present disclosure contemplates introducing part of the vector into a Brassicaceae plant cell using Agrobacterium (e.g., Agrobacterium tumefaciens). As such, the present disclosure provides an Agrobacterium host cell capable of transforming a Brassicaceae plant cell (e.g., a Brassicaceae ovule precursor cell for example) and having been transformed to comprise the first nucleic acid molecule described herewith. For example, the first nucleic acid molecule can be provided in the form of a vector as described herein. In some embodiments, the vector comprises a selection marker that allows the selection and expansion of Agrobacterium host cells having the vector and expressing the selection marker. The vector can comprise the first nucleic acid molecule (also referred to as the first transgene) encoding the Lal2 polypeptide. The first nucleic acid molecule is considered transgenic with respect to the Agrobacterium host cell. In the context of the present disclosure, a nucleic acid molecule is considered transgenic with respect to a cell (either in vivo or in vitro) because the nucleic acid molecule has been isolated from an organism that is different from the organism from which the cell is derived or located. In an embodiment, the nucleic acid molecule is considered transgenir with respect to a Brassicaceae plant or cell because it has been isolated or derived from a non-Brassicaceae plant or cell.

As such, the present disclosure provides a transgenic Brassicaceae plant or cell comprising the first nucleic acid molecule described herein. In the context of the present disclosure, the Brassicaceae plant, prior to its transformation with the first nucleic acid molecule, is self-compatible. In some embodiments, the Brassicaceae plant, prior to its transformation with the first nucleic acid molecule, can either be devoid of a Lal2 gene ortholog or can comprise a non-functional Lal2 gene ortholog (e.g, a Lal2 gene ortholog encoding a Lal2 protein which cannot confer self-sterility). In still other embodiments, the self-compatible Brassicaceae plant can express a functional SCRL polypeptide that will be recognized by the Lal2 protein encoded by the first nucleic acid molecule. Self-compatible Brassicaceae plants include, but are not limited to, Camelina (e.g., Camelina sativa), Canola and self-compatible varieties of cole crops such as cabbage, broccoli, kale, and their near relatives. Still in the context of the present disclosure, the first nucleic acid molecule is considered transgenic with respect to the Brassicaceae plant or cell because the first nucleic acid molecule has been isolated from an organism that is different from the Brassicaceae plant or cell. As indicated above, the first nucleic acid molecule can be introduced into the Brassicaceae plant or cell using the vector described herein or the Agrobacterium host cell described herein. In some embodiments, the first nucleic acid molecule is integrated in the genome of the transgenic Brassicaceae plant or cell. In yet another embodiment, the Brassicaceae plant or cell is homozygous for the first nucleic acid molecule, e.g., it bears two copies of the first nucleic acid molecule at the same genetic locus. In another embodiment, the Brassicaceae plant or cell is heterozygous for the first nucleic acid molecule, e.g., it bears a single of the first nucleic acid molecule at a defined genetic locus. In some embodiment, the Lal2 polypeptide is preferably expressed (and in additional embodiments is exclusively expressed) in the stigma of the transgenic plant. The present disclosure provides transgenic Brassicaceae plants, transgenic Brassicaceae plant parts (e.g., stigma), transgenic Brassicaceae plant cells, transgenic Brassicaceae seeds as well as transgenic Brassicaceae seed cells. The present disclosure also provides plant products (e.g., oil, feedstock) obtained from the processing of transgenic Brassicaceae plants, transgenic Brassicaceae plant parts (e.g., stigma), transgenic Brassicaceae plant cells, transgenic Brassicaceae seeds as well as transgenic Brassicaceae seed cells.

The genetic engineering of the first nucleic acid in the Brassicaceae plant will not necessarily induce self-sterility in the transgenic plant. If, prior to transformation, the Brassicaceae plant expresses a SCRL polypeptide that is recognized by the transgenic Lal2 polypeptide, then the transgenic Brassicaceae plant will be self-incompatible. However, if, prior to transformation, the Brassicaceae plant does not express a secreted SCRL polypeptide that can be recognized by the transgenic Lal2 polypeptide, then the transgenic Brassicaceae will still be self-compatible and will required to be genetically engineered or crossed to express a SCRL polypeptide that can be recognized by the transgenic Lal2 polypeptide. Examples of Brassicaceae plants that will remain self-compatible even though they express a transgenic Lal2 polypeptide (preferably in their stigma) include plants that are not capable of secreting a SCRL polypeptide, that produce a non-functional SCRL polypeptide (e.g., a truncated from of the SCRL polypeptide), that produce a functional SCRL polypeptide but non-cognate to the Lal2 polypeptide, or that do not express any SCRL polypeptides.

### SCRL Polypeptides and Associated Tools

The genetic system described herein comprises, as a second component, a nucleic acid coding for the SCRL polypeptide. In the context of the present disclosure, the SCRL polypeptide is derived from a SCRL gene that is located at most at 10 000 base pairs from its cognate Lal2 gene encoding a corresponding Lal2 polypeptide. In the context of the present disclosure, a “SCRL polypeptide” refers to a secreted polypeptide encoded by the SCRL gene and being expressed in the inner cell layers of the anther (anther tapetum) and deposited on the surface of pollen in a Brassicaceae plant. Upon specific binding to its cognate receptor Lal2, self-recognition occurs and Lal2 is capable of initiating intracellular signaling that will ultimately lead to the prevention of self-pollen hydration and growth of the pollen tube. The SCRL polypeptide comprises a signal peptide domain and an embodiment of such signal peptide is shown, in the amino acid sequence of SEQ ID NO: 72, between amino acid residues located between location 1 and 33.

In some embodiments, the SCRL polypeptide has, consists essentially of or consists of the amino acid consensus sequence of SEQ ID NO: 72. In other embodiments, the SCRL polypeptide is devoid of a signal peptide and has, consists essentially of or consists of the amino acid sequence located between residues 34 to 107 of SEQ ID NO: 72. Alternatively or in combination, the SCRL polypeptide can have, consist essentially of or consist of a polypeptide having the amino acid residues important for recognizing and binding to the Lal2 polypeptide. For example, the SCRL polypeptide can have, consist essentially of or consist of a polypeptide having at least one, and in some embodiments, two, three, four, five, six, seven or eight of any one of cysteine residues at positions corresponding to amino acid residues 56, 65, 69, 80, 89, 91, 97 SEQ ID NO: 72. These cysteine residues are characteristic of proteins belonging to the defensins gene family, a group of small secreted proteins generally involved in immunity and self-defense, and they maintain the protein structure through their difulfite bonds. In yet further embodiments, the SCRL polypeptide can have, consist essentially of or consist of the amino acid sequence of any one of SEQ ID NO: 1 and 2. In the context of the present disclosure, the polypeptides set forth in SEQ ID NO: 3 and 4 are not considered to be SCRL polypeptides.

In other embodiments, the SCRL polypeptide is encoded by an ortholog of the SCRL gene (e.g., a SCRL gene ortholog also referred to as a SCRL ortholog). In the context of the present disclosure, a “SCRL gene ortholog” is understood to be a gene in a different plant species that evolved from a common ancestral gene by speciation. Still in the context of the present disclosure, a SCRL gene ortholog encodes a polypeptide having a biological function similar to the SCRL polypeptide, e.g. it can act as a secreted protein on pollen allowing self-incompatibility in Brassicaceae. SCRL orthologs are located within 10 000 base pairs of their cognate Lal2 genes. SCRL orthologs include, but are not limited to genes encoding the polypeptides having the following GenBank Accession Number: NCBI_Gene_ID_{—}9305018 (also called AlLal2 or SEQ ID NO: 67). In the context of the present disclosure, SCRL orthologs exclude SCRL paralogs encoding polypeptides having any one of the following Genbank of EMBL Accession Numbers CCI61481.1, CCI61490.1, CCI61491.1, CCI61492.1, ADG01814.1, ACN63521.1, ADQ37355.1, ADQ37361.1, EFH53838.1, EFH59713.1, EFH59715.1, EFH59946.1, EFH60431.1, EFH62083.1, EFH62845.1, NP_{—}564768.1, NP_{—}974058.1, NP_{—}974556.1, NP_{—}001030751.1, NP_{—}001030752.1, NP_{—}001030783.1, NP_{—}001031003.1, NP_{—}001031212.1, NP_{—}001031213.1, NP_{—}001031214.1, NP_{—}001031236.1, NP_{—}001031324.1, NP_{—}001031326.1, NP_{—}001031342.1, EFH69052.1, EFH52506.1, XP_{—}002876247.1, XP_{—}002877579.1, XP_{—}002883454.1, XP_{—}002883456.1, XP_{—}002883687.1, XP_{—}002884172.1, XP_{—}002885824.1, XP_{—}002886586.1, XP_{—}002892793.1, NP_{—}171880.1, NP_{—}190990.1, NP_{—}197752.1, NP_{—}683589.1, NP_{—}195935.2, NP_{—}001030951.1, NP_{—}001031354.1, NP_{—}001031414.1, NP_{—}001031608.1, NP_{—}001031611.1, NP_{—}001031616.1, NP_{—}001031643.1, NP_{—}001031648.1, NP_{—}001031693.1, NP_{—}001031694.1, NP_{—}001031775.1, NP_{—}001031776.1, NP_{—}001031783.1, NP_{—}001032016.1, ABV21220.1, AEC05895.1, AEC05918.1, AEC06027.1, AEC06296.1, AEC07735.1, AED90561.1, AED93192.1, AED95310.1, AEE27620.1, AEE27621.1, AEE28335.1, AEE33756.1, AEE33757.1, AEE33758.1, AEE33759.1, AEE33763.1, AEE34332.1, AEE76805.1, AEE76806.1, AEE76876.1, AEE77331.1, AEE79200.1, AEE82843.1, AEE82885.1, AEE82926.1, AEE83498.1, AEE83642.1, AEE83643.1, AEE84550.1, AEE84553.1, AEE86107.1, AEE86108.1, AEE86228.1, CCO14089.1, CBK21749.2, ACN52011.1, ACN52012.1, ACN52013.1, ACN52014.1, ACN52015.1, ACN52016.1, ACN52017.1, ACN52018.1, ACN52019.1, ACN52020.1, ACN52021.1, ACN52022.1, ACN52023.1, ACN52024.1, ACN52025.1, ACN52026.1, ACN52027.1, ACN52028.1, ACN52029.1, ACN52030.1, ACN52031.1, ACN52032.1, ACN52033.1, ACN52034.1, ACN52035.1, ACN52036.1, ACN52037.1, ACN52038.1, ACN52039.1, ACN52040.1, ACN52041.1, ACN52042.1, ACN52043.1, ACN52044.1, ACN52045.1, ACN52046.1, ACN52047.1, ACN52048.1, ACN52049.1, ACN52050.1, ACN52051.1, ACN52052.1, ACN52053.1, ACN52054.1, ACN52055.1, ACN52056.1, ACN52057.1, ACN52058.1, ACN52059.1, ACN52060.1, ACN52061.1, ACN52062.1, AAF17503.1, AAF17504.1, CAC19879.1, BAC24040.1, BAC24041.1, BAC24042.1, BAC24043.1, BAC24044.1, BAC24045.1, BAC24046.1, BAC24047.1, BAC24048.1, BAC24049.1, BAC24050.1, BAC24051.1, BAC24052.1, BAC24053.1, BAC24054.1, BAC24055.1, BAC24056.1, BAC24057.1, BAC24058.1, BAC24059.1, BAC24060.1, BAC24061.1, BAC24062.1, BAC24063.1, BAC24064.1, BAC24065.1, BAC24066.1, BAC24067.1, BAC24068.1, BAC24069.1, BAC24070.1, BAC24071.1, BAC24072.1, BAC24073.1, BAC24074.1, BAC24075.1, BAC24076.1, BAC24077.1, BAC24078.1, BAC24079.1, BAC24080.1, BAC24081.1, BAC24082.1, BAC24083.1, BAC24084.1, BAC24085.1, ABQ52684.1, BAC24025.1, BAC24026.1 and BAC24027.1. In an embodiment, the degree of identity of SCRL gene ortholog with respect to the SCRL polypeptide is at least 28.3% in a MUSCLE (MUltiple Sequence Comparison by Log-Expectation) alignment (when determined on the entire open-reading frame of the SCRL gene). In another embodiment, the SCRL ortholog encodes a polypeptide having the amino acid sequence set forth in SEQ ID NO: 72.

In yet another embodiment, the SCRL polypeptides described herein also encompass SCRL polypeptide variants. In the context of the present disclosure, the “SCRL polypeptide variants” are polypeptides that vary in at least one amino acid residue when compared to the SCRL polypeptide. This variation can be the addition of an amino acid residue, the removal of an amino acid residue or the modification in the identity of an amino acid residue when compared to the SCRL polypeptide. In some embodiments, the SCRL polypeptide variant is a function-conservative variant in which a change in one or more nucleotides in a given codon position of the SCRL gene results in a SCRL polypeptide sequence in which a given amino acid residue in the polypeptide has been replaced by a conservative amino acid substitution. The SCRL polypeptide variants encode a polypeptide having the same biological function as the SCRL polypeptide, e.g. it can act as a ligand for the Lal2 receptor and allow self-incompatibility in a Brassicaceae plant. In an embodiment, the degree of identity between the amino acid sequence of the SCRL variant and the SCRL polypeptide is of 44.9% in a MUSCLE (MUltiple Sequence Comparison by Log-Expectation) alignment (when determined on the entire amino acid sequence frame of the SCRL polypeptide). Because of the nature of their role in self-recognition, the variants are expected to share a low degree of sequence identity and the classification of a sequence as being a SCRL variant can be confirmed with certainty only by determining their genomic location.

In still another embodiment, the SCRL polypeptides described herein encompass SCRL polypeptide fragments. In the context of the present disclosure, the “SCRL polypeptide fragments” are polypeptides that are at least one amino acid residue shorter than the SCRL polypeptide. For example, one contemplated SCRL fragment is devoid of a signal peptide and, in some embodiments, can have, consist essentially of or consists of the amino acid residues 34 to 107 of SEQ ID NO: 72. In some embodiments, the deletion can be located at the NH₂ terminal of the SCRL polypeptide or the COOH terminal of the SCRL polypeptide. The deletion can be between contiguous amino acids or can affect different non-contiguous amino acids. The SCRL fragments of the present disclosure do not include those presented in SEQ ID NO: 3 or 4. The SCRL polypeptide fragments have the same biological function as the SCRL polypeptide, e.g. it can act as a ligand for the Lal2 receptor. In an embodiment, the total number of amino acids in the SCRL fragment is decreased by 1%, 2%, 3%, 4%, 5%, 6%, 7%, 8%, 9% or 10% when compared to the total amino acid number of the SCRL polypeptide.

The second nucleic acid molecule of the genetic system can be derived/isolated from a genomic sequence of the SCRL gene (or the SCRL ortholog) or a corresponding transcript of such SCRL gene or SCRL ortholog. In some embodiment, the SCRL gene encodes a protein having the amino acid sequence of SEQ ID NO: 71, for example the amino acid sequence of any one of SEQ ID NO: 1 or 2. In some embodiment, the second nucleic acid molecule is a complementary DNA of a transcript of the SCRL gene or SCRL ortholog. In other embodiments, the second nucleic acid molecule is derived from the amplification of the genomic sequence of the SCRL gene or SCRL ortholog or of a transcript (for example a messenger RNA transcript) expressed from the SCRL gene or SCRL ortholog. In one embodiment, the oligonucleotides used to amplify the genomic sequence of the SCRL gene or SCRL ortholog or of a transcript expressed from the SCRL gene or SCRL ortholog can be those set forth in Table 1 (for example, a1-1 SCRL variant: SCR_TNC_F1 & SCR_TNC_R1; a1-2 and a2 SCRL variants: SCR_A2_F3 & SCR_A2_R3, A2_gSCR_F1 & A2_gSCR_R3; a4 variant: SCR_Rus_{—}2F & SCR_Rus_{—}2R; Al_SCRL_Exon1_F & Al_SCRL_Exon2_R).

The second nucleic acid molecule of the genetic system can be included in a vector intended to be used to produce a transgenic Brassicaceae. For example, the second nucleic acid molecule can be used as a transgene and included in an appropriate vector. In some embodiment, the vector can also comprise a promoter operatively linked to the transgene encoding the transgenic SCRL polypeptide. The promoter can be constitutive or regulated. In some embodiment, the promoter can allow for the expression of the transgene more preferably (and in some embodiments exclusively) in the anther of a Brassicaceae plant or a Camelina plant. Alternatively, the promoter is active in the anther tapetum, e.g., it allows for the expression of genes in the anther tapetum. Such anther-specific and anther-active promoters include, but are not limited to the ATA7 anther-specific promoter (Tsuchimatsu T. et al., Nature 2010) and the SCRL variant native promoter, the TA29 promoter, the tapetum-specific A6 promoter, the A. thaliana tapetum-specific A9, the Sta 41-2 and Sta 41-9 promoters (renamed BnOlnB; 3 and BnOlnB; 4 respectively, Hong, H. P. et al., Plant Mol. Biol. 34:549-555 (1997b)), the putG1, atgrp-6, -7 and -8 promoters (renamed AtOlnB; 1, 2, 3 and 4, de Oliveira, D. E. et al., Plant J 3:495-507 (1993)), the 13 promoter (renamed BnOlnB; 1 Roberts, M. R. et al., Plant Mol. Biol. 17:295-299 (1991)), the C98 promoter (renamed BnOlnB; 2, Hodge, R. et al., Plant J 2:257-260 (1992)), the Pol3 promoter (renamed BnOlnB; 5, Roberts, M. R. et al., Planta 195:469-470 (1995)), the bopc4 promoter (renamed BoOlnB; 1, Ruiter, R. K. et al., Plant Cell 9:1621-1631 (1997)), the BrOlnB1, 2, 3, 4 and 5 promoters (Lim et al. (1994) EMBL Acc. No. L33510, L33543, L33564, L33603, L33618), the BnOlnB; 6, 7, 8, 9, 10, 11 and 12 promoters (Ross, J. H. E. & Murphy, D. J., Plant J. 9:625-637 (1996)), the LeFRK4 promoter, the Bnml promoter, the tapetum-specific promoter hybridizable to TA29, TA26 or TA13. In additional embodiments, when the SCRL polypeptide does not have a signal peptide, the vector can also include, upstream of the SCRL transgene, and operatively linked to the SCRL transgene, a nucleic acid molecule encoding a signal peptide which lead to the secretion of the transgenic SCRL polypeptide on the surface of inner cell layers of the anther (anther tapetum). Embodiments of such signal peptide include, but are not limited to, the signal peptide of amino acid residues 1 to 33 of SEQ ID NO: 72. In further embodiments, the vector can also comprise a selection marker that can allow for the identification of cells (e.g., Agrobacterium cells or plant cells) comprising the vector or part of the vector. In yet another embodiment, the vector can be designed to be partly integratable into the genome of a recipient cell (such as a Brassicaceae cell). For example, the vector can be designed to be partly integrated into an Agrobacterium cell as well as partly integretable/integrated in the genome of a plant cell (such as a Brassicaceae cell). In such embodiment, the vector that is to be introduced into the Agrobacterium cell comprises an Agrobacterium selection marker and the part of the vector that is to be integrated in the plant cell comprises a plant selection marker. In additional embodiments, the vector can be designed to be able to replicate independently in an Agrobacterium host cell.

In still another embodiment, the second nucleic acid molecule or the second vector can be operably linked to the first nucleic acid molecule (encoding the Lal2 polypeptide or a variant thereof, described below) or the first vector (comprising the second nucleic acid molecule). In some embodiments, the first and the second nucleic acid molecule may be included in a single vector that may be suitable for expansion in Agrobacterium and, in yet other embodiments, for integration in the Brassicaceae plant or cell.

Although other plant transformation techniques are contemplated, the present disclosure contemplates introducing part of the vector into a Brassicaceae plant cell using Agrobacterium (e.g., Agrobacterium tumefaciens). As such, the present disclosure provides an Agrobacterium host cell capable of transforming a Brassicaceae plant cell (e.g., a Brassicaceae ovule precursor cell) and having been transformed to comprise the second nucleic acid molecule described herewith. For example, the second nucleic acid molecule can be provided in the form of a vector as described herein. In some embodiments, the vector comprises a selection marker that allows the selection and expansion of Agrobacterium host cells having the vector and expressing the selection marker. The vector can comprise the second nucleic acid molecule (also referred to as the second transgene) encoding the SCRL polypeptide. The second nucleic acid molecule is considered transgenic with respect to the Agrobacterium host cell. In the context of the present disclosure, a nucleic acid molecule is considered transgenic with respect to a cell (either in vivo or in vitro) because the nucleic acid molecule has been isolated from an organism that is different from the organism from which the cell is derived or located.

As such, the present disclosure provides a transgenic Brassicaceae plant or cell comprising the second nucleic acid molecule described herein. In the context of the present disclosure, the Brassicaceae plant or cell, prior to its transformation with the second nucleic acid molecule, is self-compatible. In some embodiments, the Brassicaceae plant, prior to its transformation with the second nucleic acid molecule, can either be devoid of a SCRL gene ortholog or can comprise a non-functional SCRL gene ortholog (e.g., a SCRL gene ortholog encoding a SCRL protein which cannot confer self-sterility). In yet another embodiment, the Brassicacea plant can express a Lal2 polypeptide or a variant thereof as described above. Self-compatible Brassicaceae plants include, but are not limited to, Camelina (e.g., Camelina sativa), Canola and self-compatible varieties of cole crops such as cabbage, broccoli, kale, and their near relatives. Still in the context of the present disclosure, the second nucleic acid molecule is considered transgenic with respect to the Brassicaceae plant or cell because the second nucleic acid molecule has been isolated from an organism that is different from the Brassicaceae plant or cell. As indicated above, the second nucleic acid molecule can be introduced into the Brassicaceae plant or cell using the vector described herein or the Agrobacterium host cell described herein. In some embodiment, the second nucleic acid molecule is integrated in the genome of the transgenic Brassicaceae plant or cell. In yet another embodiment, the Brassicaceae plant or cell is homozygous for the second nucleic acid molecule, e.g., it bears two copies of the second nucleic acid molecule at the same genetic locus. In yet another embodiment, the Brassicaceae plant or cell is heterozygous for the second nucleic acid molecule, e.g., it bears a single copy of the second nucleic acid molecule at a defined genetic locus. In some embodiments, the SCRL polypeptide is preferably expressed (and in additional embodiments is exclusively expressed) in the anther tapetum of the transgenic plant and is secreted on the pollen of the transgenic plant. The present disclosure provides transgenic Brassicaceae plant, transgenic Brassicaceae plant parts (e.g., anther), transgenic Brassicaceae plant cell, transgenic Brassicaceae seed as well as transgenic Brassicaceae seed cells. The present disclosure also provides plant products (e.g., oil, feedstock) obtained from the processing of transgenic Brassicaceae plants, transgenic Brassicaceae plant parts (e.g., anther), transgenic Brassicaceae plant cells, transgenic Brassicaceae seeds as well as transgenic Brassicaceae seed cells.

The genetic engineering of the second nucleic acid in the Brassicaceae plant will not necessarily induce self-incompatibility in the transgenic plant. If, prior to transformation, the Brassicaceae plant expresses a Lal2 polypeptide that recognizes the transgenic SCRL polypeptide, then the transgenic Brassicaceae plant will be self-incompatible. However, if, prior to transformation, the Brassicaceae plant does not express a Lal2 polypeptide that can recognize the transgenic SCRL polypeptide, then the transgenic Brassicaceae will still be self-compatible and will require to be genetically engineered or crossed to express a Lal2 polypeptide that recognizes the transgenic SCRL polypeptide. Examples of Brassicaceae plants which will remain self-compatible even though they express a transgenic SCRL polypeptide (preferably in their anthers) include plants that are not capable of localizing a Lal2 polypeptide on the stigma surface, that produce a non-functional Lal2 polypeptide, that produce a functional Lal2 polypeptide but non-cognate to the SCRL polypeptide, or that do not express any Lal2 polypeptides.

### Genetic System and Methods for Providing Self-Incompatibility

The present disclosure provides methods as well as associated genetic systems to introduce sporophytic self-incompatibility in a Brassicaceae plant that is otherwise self-compatible. In order to achieve this goal, the self-compatible Brassicaceae plants must be genetically engineered (and optionally crossed) to express a functional Lal2 polypeptide and a functional corresponding SCRL polypeptide and to exhibit rejection of self-pollen. Initially, and optionally, the method can comprise selecting a self-compatible Brassicaceae plant and characterizing the S locus (at the DNA, RNA or polypeptide level) to determine if the selected plant has a Lal2 gene (or Lal2 gene ortholog) and/or expresses a functional Lal2 polypeptide (which, upon binding to its corresponding SCRL polypeptide can induce self-incompatibility), has a SCRL gene (or a SCRL gene ortholog) and/or expresses a functional SCRL polypeptide (which is secreted and can bind its corresponding Lal2 polypeptide to ultimately induce the self-incompatibility response). This initial characterization may guide the further manipulations that will be required to induce self-sterility in the selected plants. For example, if it is determined that the selected Brassicaceae plant has a Lal2 gene (or Lal2 gene ortholog) and expresses a functional Lal2 polypeptide but lacks a SCRL gene (or a SCRL gene ortholog) or does not express a functional SCRL polypeptide, then it is concluded that the introduction of a transgenic cognate SCRL-encoding nucleic acid is required to provide self-incompatibility. On the other hand, if it is determined that the selected Brassicaceae plant does not have a Lal2 gene (or Lal2 gene ortholog) nor express a functional Lal2 polypeptide but has a SCRL gene (or a SCRL gene ortholog) or expresses a functional SCRL polypeptide, then it is concluded that the introduction of a transgenic cognate Lal2-encoding nucleic acid is required to provide self-incompatibility. In yet another example, if it is determined that the selected Brassicaceae plant does not have a Lal2 gene (or Lal2 gene ortholog) or a SCRL gene (or a SCRL gene ortholog) nor express a functional Lal2 polypeptide or a functional SCRL polypeptide, then it is concluded that the introduction of a first transgenic Lal2-encoding nucleic acid and a cognate second transgenic SCRL-encoding nucleic acid are required to provide self-incompatibility.

In a preliminary step, the method can comprise making two different sets of independent transgenic plants out of self-compatible Brassicaceae individuals. The first set of transgenic plants comprises the first nucleic acid molecule described herein and expresses a transgenic Lal2 polypeptide at least in the stigmas. The second set of independent transgenic plants comprises the second nucleic acid molecule described herein and expresses a transgenic SCRL polypeptide at least in the anthers. Care should be taken in selecting the variants of Lal2 and SCRL polypeptides that are being introduced in the Brassicaceae plants so that the selected variants of Lal2 and SCRL can specifically bind to one another and allow signaling (e.g., phosphorylation) through the Lal2 polypeptide leading to self-incompatibility. The nucleic acid molecule encoding the Lal2 and/or SCRL polypeptides can be of any origin and can be derived from genomic DNA or a transcript of the genomic DNA (cDNA for example).

Alternatively, the method can comprise making a single independent transgenic and self-incompatible Brassicaceae plant by introducing a single nucleic acid molecule encoding both the Lal2 and the SCRL polypeptides. The transgenic plant expresses a transgenic Lal2 polypeptide at least in the stigmas and a transgenic SCRL polypeptide at least in the anthers. Care should be taken in selecting the variants of Lal2 and SCRL polypeptides that are being introduced in the Brassicaceae plants so that the selected variants of Lal2 and SCRL can specifically bind to one another and allow signaling (e.g., phosphorylation) through the Lal2 polypeptide leading to self-incompatibility. The nucleic acid molecule encoding the Lal2 and/or SCRL polypeptides can be of any origin and can be derived from genomic DNA or a transcript of the genomic DNA (cDNA for example).

In the embodiments in which it was determined or decided to introduce a first transgenic Lal2-encoding nucleic acid and a second transgenic SCRL-encoding nucleic acid in the selected Brassicaceae plant to provide self-incompatibility, the method comprises providing and crossing two transgenic Brassicaceae plants. The first transgenic Brassicaceae plants are either hemizygous for the Lal2 transgene (to use in crosses to test for the self-incompatibility response) or homozygous for the Lal2 transgene (to allow the transmission of the Lal2 transgene to all their progeny when generating double-transgenic Brassicaceae lines (see below)). Embodiments of the first transgenic Brassicaceae plants expressing a transgenic Lal2 polypeptide are provided herein. The second transgenic Brassicaceae plants are either hemizygous for the SCRL transgene (to use in crosses to test for the self-incompatibility response) or homozygous for the SCRL transgene (to allow the transmission of the SCRL transgene to all their progeny when generating double-transgenic Brassicaceae lines (see below). Embodiments of the second transgenic Brassicaceae plants expressing a transgenic SCRL polypeptide are provided herein. Crosses are conducted between the first hemizygous/homozygous transgenic lines, as pollen recipient parents, and the second hemizygous/homozygous transgenic lines, as pollen donor parents, in all possible pairwise combinations. The pairwise combinations giving the highest levels of self-incompatibility response (expected 100% or near 100% self-incompatibility) will be used to generate self-incompatible double-transgenic Brassicaceae plants. First, transgenic Brassicaceae hemizygous/homozygous for Lal2 or SCRL transgenes will be obtained from the aforementioned selected hemizygous transgenic lines by selecting among the seeds obtained from self-fertilization. Then, Brassicaceae plants double-transgenics for the transgenic Lal2 polypeptide and the transgenic SCRL polypeptide can be obtained by crossing the selected homozygous transgenic Lal2 plants, used as pollen donor parents, and the selected homozygous transgenic SCRL plants, used as pollen recipient parents (the reverse cross being expected to be self-incompatible). All the Brassicaceae seeds obtained from these crosses are expected to bear a seedling hemizygous for both the transgenic Lal2 polypeptide and the transgenic SCRL polypeptide. Once the crosses have been made, the method also comprises identifying the crossed transgenic Brassicaceae as being self-incompatible. Such identification can be made at the nucleic acid, the polypeptide level or the functional level. When the identification is made at the nucleic acid level, this step can include determining if the crossed Brassicaceae carries the first transgene (e.g., first nucleic acid encoding the Lal2 polypeptide) and the second transgene (e.g., second nucleic acid encoding the SCRL polypeptide). When the identification is made at the polypeptide level, this step can include determining if the transgenic Lal2 and the transgenic SCRL are expressed in the double-transgenic Brassicaceae and optionally where the Lal2 polypeptide and the SCRL polypeptide are expected to be expressed in the plant (stigma for the transgenic Lal2 and anther for the transgenic SCRL). When the identification is made at the functional level, this step can include determining the level of self-compatibility or self-incompatibility in the double-transgenic Brassicaceae plants.

The present disclosure thus also provide a self-incompatible transgenic Brassicaceae plant or cell derived therefrom having a first transgene comprising the first isolated nucleic acid molecule encoding the Lal2 polypeptide and a second transgene comprising the second isolated nucleic acid molecule encoding the SCRL polypeptide. The transgenic Brassicaceae plant can be hemizygous or homozygous for the first Lal2 transgene and the second SCRL transgene. The present disclosure also provides Brassicaceae plant parts (e.g., anther, stigma, pollen, etc.), transgenic Brassicaceae plant cells, transgenic Brassicaceae seeds as well as transgenic Brassicaceae seed cells. The present disclosure also provides plant products (e.g., oil, feedstock) obtained from the processing of transgenic Brassicaceae plants, transgenic Brassicaceae plant parts, transgenic Brassicaceae plant cells, transgenic Brassicaceae seeds as well as transgenic Brassicaceae seed cells. The transgenic Brassicaceae plant can be from any Brassicaceae species and specifically includes Camelina plants.

In order to perform such methods, the present disclosure also provides a genetic system with tools that may be required to obtain the self-incompatible Brassicaceae plant. The genetic system comprises at least one transgenic Lal2 element and/or at least one transgenic SCRL element. Care should be taken in selecting the variants of Lal2 and SCRL polypeptides encoded or expressed by the Lal2 or SCRL elements so that the selected variants of Lal2 and SCRL can specifically bind to one another and allow signaling (e.g., phosphorylation) through the Lal2 polypeptide. Contemplated transgenic Lal2 elements include, but are not limited to the first isolated nucleic acid (encoding the Lal2 polypeptide) described herein, the first vector comprising the first isolated nucleic acid (encoding the Lal2 polypeptide) described herein, the first transgenic Agrobacterium host cell comprising the first isolated nucleic acid or the first vector, and the first transgenic Brassicaceae plant or cell (expressing the transgenic Lal2 polypeptide) described herein. A single Lal2 element or any combinations of Lal2 elements can be provided in the genetic system.

Contemplated SCRL elements include, but are not limited to, the second isolated nucleic acid (encoding the SCRL polypeptide) described herein, the second vector comprising the second isolated nucleic acid molecule (encoding the SCRL polypeptide) described herein, the second transgenic Agrobacterium host cell comprising the second isolated nucleic acid molecule or the second vector described herein and the second transgenic Brassicaceae plant or cell (expressing the SCRL polypeptide) described herein. A single SCRL element or any combinations of SCRL elements can be provided in the genetic system. The genetic system described herein can also comprise any combination of at least one Lal2 element with at least one SCRL element. For example, the genetic system can comprise a vector encoding the transgenic Lal2 polypeptide and a transgenic Brassicaceae cell expressing the SCRL polypeptide. The genetic system can also comprise instructions on how to use the Lal2 elements and/or the SCRL elements to provide a self-incompatible Brassicaceae plant.

### Methods for Producing Brassicaceae Hybrids

Once a self-incompatible Brassicaceae plant has been obtained it can be used as a pollen recipient parent to produce a Brassicaceae hybrid. As indicated herein, producing hybrid from self-compatible Brassicaceae plants can be tedious and cannot be scaled-up. As such, in order to produce a Brassicaceae hybrid from a Brassicaceae self-compatible variety, it is advantageous to use the isolated nucleic acids (as well as the related products), the genetic system and the methods described herewith.

In order to produce a hybrid Brassicaceae, the method first involves crossing the self-incompatible transgenic Brassicaceae plant, used as a pollen recipient parent, with a second Brassicaceae plant (of a variety different from the self-incompatible transgenic Brassicaceae plant), used as a pollen donor parent, so as to provide a hybrid Brassicaceae plant. The second Brassicaceae plant can be self-compatible and can effectively fertilize or be fertilized by the self-incompatible transgenic Brassicaceae plant. Once a hybrid Brassicaceae plant has been obtain, the present method also comprises identifying it as a hybrid Brassicaceae. For example, the hybrid Brassicaceae plant could be identified as exhibiting traits unique to each parent.

The method can also comprise restoring self-compatibility in the hybrid plant by inhibiting or down regulating the expression of the SCRL and/or the Lal2 polypeptides. Such inhibition can be achieved, for example, by introducing in the pollen donor parental variety, a silencing RNA (siRNA) construct to specifically target the silencing of the SCRL and/or Lal2 variants introduced in the double-transgenic self-incompatible Brassicaceae plant mentioned above, the latter being used as the pollen recipient parental line. The siRNA construct, for example, consists of a fragment of the Lal2 and/or SCRL sequence(s) variants (such as those described in Table 1) cloned as inverted repeats separated by a short DNA spacer and operationally linked to a stigma and/or an anther tapetum active promoter(s). The siRNA construct is in the homozygous state in the pollen donor parental line and is as such transmitted by the pollen to all the hybrid progeny. By silencing the expression of Lal2 and/or SCRL, self-fertility is restored in the hybrid individuals that inherited both the Lal2 and the SCRL transgenes from the pollen recipient parental line.

The present disclosure further provides a hybrid Brassicaceae plant or cell as described herein. In some embodiment, the hybrid Brassicaceae plant is a Camelina plant. The hybrid Brassicaceae plant can be transgenic for the nucleic acid molecules encoding the Lal2 and the SCRL polypeptides. In some embodiments, the hybrid Brassicaceae plant can be produced by the method described herein. The present disclosure provides for hybrid Brassicaceae plants, hybrid Brassicaceae plant parts, hybrid Brassicaceae plant cells, hybrid Brassicaceae seeds as well as hybrid Brassicaceae seed cells. The present disclosure also provides plant products (e.g., oil, feedstock) obtained from the processing of hybrid Brassicaceae plants, hybrid Brassicaceae plant parts, hybrid Brassicaceae plant cells, hybrid Brassicaceae seeds as well as hybrid Brassicaceae seed cells.

The present invention will be more readily understood by referring to the following examples that are given to illustrate the invention rather than to limit its scope.

### Example I

### Cloning and Characterization of Leavenworthia Lal2 and SCRL Genes

Plant material and growth conditions. Leavenworthia alabamica seed was sown in a 1:1 mixture of PRO-MIX BX™ (Quebec, Canada) and sand. Plants used for expression analyses, genome sequencing and fosmid cloning were grown in a Conviron PGW36 growth chamber under 14 h days at 22° C. with a nighttime temperature of 18° C. Plants used for crossing were grown in a greenhouse at a minimum daytime temperature of 20° C. and 18° C. at night. Supplemental lighting was provided as needed to achieve a minimum day length of 12 h.

When generating plants for expression analyses and crossing, plants homozygous for functional S-locus haplotypes (a1-1 and a1-2) were generated through self-pollination using a saline treatment modified from Carafa et al. (1997). The stigma of the plant to be selfed was hydrated with 0.5 M NaCl. After 1 hr the stigma was then pollinated with self-pollen, either from an anther from the same flower or from another open flower of the same plant. The resulting progeny were screened for homozygosity for the allele of interest. Plants from the a2 and a4 races of L. alabamica are homozygous for the a2 and a4 LaLal2 S haplotypes, respectively. Crosses and pollen tube staining were conducted according to previously published methods Busch et al. (2008). Pollinations were considered compatible when more than 5 pollen tubes were visible in the style of the maternal parent or >1 seed was produced in the mature silique.

The Arabidopsis lyrata plant used in AlLal2 and AlSCRL expression analysis was obtained from a seed collected in KivimäKi et al. (2007) and was grown in a Conviron growth chamber in the same condition as stated above but with a 16 h period of light.

Nuclei purification and DNA extraction. Genomic DNA samples of plants containing the a1-1, a2 and a4 S haplotypes used in fosmid library construction were extracted from purified nuclei. Nuclei were purified from fresh or frozen plant tissues. Tissues were grinded in liquid nitrogen using a mortar and pestle. Powdered tissues were added to freshly made and ice-cold nuclei extraction buffer [10 mM Tris HCl (pH 9.5); 10 mM EDTA (pH 8.0); 100 mM KCl; 500 mM sucrose; 4 mM spermidine; 1 mM spermine; 0.1% β-mercaptoethanol] in a ratio of 20 ml of buffer per gram of tissue. Solution with added tissue was stirred using a magnetic stir bar for 10 min and then filtered through two layers of cheesecloth combined to one layer of Miracloth into a clean beaker. Cold lysis buffer (nuclei extraction buffer with 10% Triton X-100) was added at a ratio of 2 ml per 20 ml of nuclei extraction buffer. Solution was stirred for 2 min before pouring into cold 50 ml polyethylene tubes followed by centrifugation at 2000 g for 10 minutes at 4° C. to pellet nuclei. Supernatant was poured off and remaining supernatant was removed with a micropipette after a quick-spin.

DNA was extracted from purified nuclei using Genomic-tips 20/G and the Genomic DNA Buffer Set (Qiagen). Instructions given in the Qiagen Genomic DNA Handbook (August 2001) for Yeast starting at p. 37, step 8 were used except for this following modification: at step 9, Proteinase K was added and incubation was carried overnight with gentle shaking at 50 rpm on an MixMate Plate and Tube Mixer (Eppendorf) to lyse the nuclei. Genomic DNA used in standard DNA analysis was extracted with DNeasy Plant Mini Kit (Qiagen).

Fosmid Library Construction and Screening.

Fosmid libraries were constructed using the CopyControl™ HTP Fosmid Library Production Kit (Epicentre Biotechnologies) as specified by the manufacturers instructions with the following modifications and specifications. Genomic DNA was sheared by passing gDNA samples 35 times through a Gastight 10 μl Hamilton syringe (model 1701). Sheared DNA was end-repaired and submitted to size separation by migration on a 1% low melting point agarose gel for 36 hours at 35V in 0.5× TBE buffer. Insert DNA ranging from 23 to 40 kb was recovered from the gel matrix using GELase. 250 μg of purified DNA was used for ligation into the pCC2FOS™ Vector. After titering the packaged fosmid clones, cells were grown overnight at 37° C. in liquid gel pools Elsaesser et al. (2004), Hrvatin (2007) in 96-deep-well plates at a density of either 100 or 250 cfu per pool in 200 μl of LB SeaPrep® Agarose (Lonza Rockland Inc.) supplemented with 12.5 μg/ml chloramphenicol (Cam).

Clones containing the Lalal2 gene were isolated by doing successive rounds of PCR screening on library pools of decreasing number of clones. In the first round, an aliquot of several library pools were combined to create superpools. Cells were pelleted by centrifugation and resuspended in sterile water. An aliquot of 0.5 μl each of resuspended cells was used in standard PCR reactions. In the second round, pools from the obtained positive superpools were screened. In the third round, positive pools were plated on LB agar plates supplemented with 12.5 μg/ml Cam to get isolated colonies. Colonies were individually picked and combined into pools of ten colonies for PCR screening. Final screening round was carried on individual colonies grown on LB agar containing 12.5 μg/ml choramphenicol from positive pools of ten.

To increase sensitivity of the screening, each round of screening consisted of two successive rounds of PCR reaction (primary and secondary). Primary PCR reactions were carried with primer pair Lal-Sdomain5′-F and Lal-Sdomain3′-R. Secondary PCR reaction used nested primer pair LalGenF and LalRcon (See Table 1 for primer sequences).

TABLE 1
Nucleic acid sequences of
the primers used in Example I

| | | | | SEQ ID |
|---|---|---|---|---|
| | Name | Dir. | Sequence (5′-3′) | NO.: |
| LaI2 fosmid | LaI-Sdomain5′-F1 | F | ACCTTTGGTGGCAGAGCTTC | 24 |
| library screen | | | | |
| primary PCR | LaI-Sdomain3′-R | R | AATGCTGTACAGTTGCAATTC | 25 |
| LaI2 fosmid | LaIGenF | F | TTCTATGGCAGAGCTTTGA | 26 |
| library screen | | | | |
| secondary | LaIRcon | R | ACYTCTTCTCRCATTCTTCC | 27 |
| nested PCR | | | | |
| a1-1_LaLaI2 | TNC_LaI2_Exon1-F | F | AAGTTACAACACCGATGAGG | 28 |
| RT-PCR | | | | |
| expression pattern | LaI2_Exon7-R1 | R | AGTACAGGATCTACTATCTC | 29 |
| LaLaI2 RT-PCR | LaI2-Exon5-F1 | F | ACCAAGATTCTCGGTTTAGG | 30 |
| Stigma (-2) | LaI2-Exon7-R1 | R | AGTACAGGATCTACTATCTC | 31 |
| expression | | | | |
| LaLaI2 5′ RACE | 5′RACE outer | F | GCTGATGGCGATGAATGAACACTG | 32 |
| primary PCR | LaI2_5′RACE_R1 | R | AGCACGAAATTGCCGTTATC | 33 |
| LaLaI2 5′ RACE | 5′RACE inner | F | CGCGGATCCGAACACTGC | 34 |
| | | | GTTTGCTGGCTTTGATG | |
| secondary PCR | LaI2_5′RACE_R2 | R | AATTGCCGTTATCCAGAAGC | 35 |
| LaLaI2 3′ RACE | LaI2_Exon6_F | F | TTGAAATTGTCAGTGGCAAG | 36 |
| primary PCR | 3′RACE outer | R | GCGAGCACAGAATTAATACGACT | 37 |
| LaLaI2 3′ RACE | LaI2_Exon7_F | F | AGATAGTAGATCCTGTACTC | 38 |
| secondary PCR | 3′RACE inner | R | CGCGGATCCGAATTAATACG | 39 |
| | | | ACTCACTATAGG | |
| a1-1 SCRL | SCR_TNC_F1 | F | AATGGCCAAAAGTGTATGGC | 40 |
| RT-PCR | | | | |
| | SCR_TNC_R1 | R | GGAAACATGAGATGAGCAAC | 41 |
| a1-2 and a2 SCRL | SCR_A2_F3 | F | ATGGCTAAAAGTGTAAGGC | 42 |
| RT-PCR | SCR_A2_R3 | R | TTATAGAGCACCAACAAAGG | 43 |
| a4 SCRL RT-PCR | SCR_Rus_2F | F | AACAGGTAAGTCTTGTTAACTTC | 44 |
| | SCR_Rus_2R | R | TTCCAACAATTTACTCTAAAGC | 45 |
| a1-1 SCRL | 5′RACE outer | F | see above | 32 |
| 5′ RACE | | | | |
| primary PCR | SCR_TNC_R1 | R | GGAAACATGAGATGAGCAAC | 46 |
| a1-1 SCRL | 5′RACE inner | F | see above | 34 |
| 5′ RACE | | | | |
| secondary PCR | SCR_TNC_R2 | R | AACAAGGCCTTACTCTGCAG | 47 |
| a1-1 SCRL | SCR_TNC_F1 | F | see above | 40 |
| 3′ RACE | | | | |
| primary PCR | 3′RACE outer | R | see above | 37 |
| a1-1 SCRL | SCR_TNC_F2 | F | TGGCTTACTAGTTTCATCAG | 48 |
| 3′ RACE | | | | |
| secondary PCR | 3′RACE inner | R | see above | 39 |
| a1-2 & a2 SCRL | 5′RACE outer | F | see above | 32 |
| 5′ RACE | | | | |
| primary PCR | SCR_A2_R3 | R | see above | 43 |
| a1-2 & a2 SCRL | 5′RACE inner | F | see above | 34 |
| 5′ RACE | | | | |
| secondary PCR | SCR_A2_R4 | R | TTTCCTTGTGGGGAACTTTC | 49 |
| a1-2 & a2 SCRL | SCR_A2_F3 | F | see above | 42 |
| 3′ RACE | | | | |
| primary PCR | 3′RACE outer | R | see above | 37 |
| a1-2 & a2 SCRL | SCR_A2_F4 | F | GCTTCATCATCTATCTAACG | 50 |
| 3′ RACE | | | | |
| secondary PCR | 3′RACE inner | R | see above | 39 |
| ARK3-Ubox region | ARK3_2eF | F | CTCCAAAGATCTCGGATTTC | 51 |
| primary PCR | PUB8_2eR | R | CGTTAACAGAGTAGCAGCAA | 52 |
| ARK3-Ubox region | ARK3_3eF | F | TGGTCTCTTGTGTGTTCAAG | 53 |
| secondary | PUB8_3eR | R | AAGCTTGGGATAGAGACTGA | 54 |
| nested PCR | | | | |
| Actin RT-PCR | Actin F | F | TATGCACTTCCACATGCTAT | 55 |
| | Actin R | R | CTTTGCGATCCACATCTGCTG | 56 |
| a1-2 SCRL allele | A2_gSCR_F1 | F | TTGTGTTGACATGGTTGCAGG | 57 |
| | A2_gSCR_R3 | R | TTGTGTTGTTATTAAGAGGG | 58 |
| a1-2 LaI2 allele | LaI2_Sdomain5′-F2 | F | TTCTATGGCAGAGCTTTG | 59 |
| | LaI2-Exon7-R1 | R | see above | 29 |
| A. lyrata SCRL | AI_SCRL_Exon1_F | F | TAGCTTCTTCATCACTTTGG | 60 |
| RT-PCR | | | | |
| & polymorphism | AI_SCRL_Exon2_R | R | TATCTTCCTTTCGGAGTAGC | 61 |
| A. lyrata LaI2 | AI_LaI2_Exon1_F1 | F | TTCCAGCCTTGACACGTATC | 62 |
| RT-PCR | | | | |
| | AI_LaI2_Exon7_R2 | R | TAAGCCGATCTGTACGCATC | 63 |
| A. lyrata LaI2 | AI_LaI2_Exon1_F | F | TTCTTCAAACCTGCAACGAG | 64 |
| polymorphism | AI_LaI2_Exon2_R | R | ACAAGTAACAAACAGCCTCC | 65 |

RNA Extraction and Expression Analysis.

Total RNA samples were extracted from plant tissues by using the RNeasy™ Plant Mini Kit (Qiagen). RNA samples were purified from DNA contamination by carrying an on-column treatment with DNase as specified in the manufacturers instruction manual. For expression analysis of Lal2 and SCRL by RT-PCR, 1 ug of total RNA was used in reverse transcription reactions with the SuperScript II Reverse Transcriptase (Invitrogen, Burlington, ON) and Oligo(dT)₁₂₋₁₈ as primer. The 5′/3′ RACE reactions were carried with the FirstChoice™ RLM-RACE Kit (Invitrogen) using 2 ug of total RNA. The 5′ adapter-ligated RNA was reverse transcribed with the M-MLV Reverse transcriptase provided with the kit and using either random decamers or the 3′ RACE adapter as primers. PCR amplifications on reverse-transcribed products were carried using the following conditions: 1 μl RT products; 1× PCR buffer; 0.2 mM dNTP mix; 2 mM MgCl₂; 0.4 μM forward primer; 0.4 μM reverse primer; 0.75 U Taq Polymerase (Invitrogen), in a final volume of 20 μl. PCR cycling was done on a C1000 thermal cycler (Bio-Rad) using the following program: initial denaturation at 94° C., 5 min. followed by 35 cycles at 94° C., 30 sec; 58° C., 30 sec.; 72° C., 1 min. and a final elongation step at 72° C., 5 min (See Table 1 for primer sequences).

Illumina RNAseq reads from A. lyrata seedlings, roots, and stage 12 flowerbuds obtained courtesy (Dr. Richard Clark and Joshua Steffen) were obtained using methods described in Gan et al. (2011). RNAseq reads were aligned to the A. lyrata reference genome (strain MN47: JGI) using both novoalign (Novocraft) and spliceMap (PMID: 20371516). Novoalign was used in read quality re-calibration mode with a low level of mismatch permitted (t=50) between read and reference. Independently spliceMap was used to map reads spanning exon junctions. For each gene model an expression level was determined by adjusting the read-count per gene by the exon-length and total reads in the respective sequencing libraries.

DNA Sequencing and Sequence Analysis.

Sanger, Illumina and 454 sequencing were performed at the McGill University and Genome Quebec Innovation Centre. The genomes of Leavenworthia alabamica, Sisymbrium irio, and the Leavenworthia short read data were gathered as part of an ongoing comparative genomics investigation involving these and other Brassicaceae species (unpublished data). The genomes of the a2 and a4 fosmids were also assembled from 454 data. In the case of the genomes, reads were generated in accordance with the Illumina protocols, with special attention paid to gentle shearing of mate-pair circular DNA to ensure >500 nt fragments, thereby reducing the probability of a read fragment-join chimera. Paired end (2×10⁵, nominal 64 nt gap) Illumina reads were generated to a depth of 80× for each genome, trimmed for quality (3′ trimming where Q<32) and assembled with the Ray assembler Boisvert et al. (2010) using automatic coverage depth profiling and a Kmer of 31. Scaffolding of Ray contigs was then undertaken with the SOAPdeNovo (BGI) assembler using a combination of 5 and 10K Base mate pair reads (unpublished data). Assembly of the fosmid sequences was undertaken in batches of pooled barcoded libraries covered by ⅛ of a flowcell of 454 sequencing (200× coverage). After stripping vector contaminants Newbler (Roche) was used to assemble the reads into ˜40 Kbase contigs using essentially default assembly parameters. Comparison of targeted fomsmid assemblies (454) and short read whole genome assemblies (Illumina-Ray) from Russelville demonstrated high levels of concordance.

Standard sequence analyses were done using the Geneious v. 5.4.6 software (Auckland, NZ) Drummond et al. (2011). Amino acid and nucleotide sequences were aligned with MUSCLE [76]. Fosmid sequences were aligned using VISTA Frazer et al. (2004)]. Annotation of fosmid sequences was done by sequence blast against the Arabidopsis thaliana genome. Because of the high sequence diversity of LaSCRL, this gene could not be detected by blast search but was found by eye examination of short ORFs obtained from different translation frames for the presence of eight cysteines. The Mauve Genome Alignment software v. 2.2.0 Darling et al. (2010) was used to compare the S locus of A. thaliana with syntenic genome region of Leavenworthia and the S locus of Leavenworthia with syntenic genome region of A. lyrata. Protein domains were determined by submitting the Lal2 and SRK amino acid sequences to the SMART/Pfam prediction tools Letunic et al. (2011).

### Phylogenetic Analyses.

In addition to the a1-1, a2 and a4 LaLal2 sequences, full-length coding SRK were selected, and the closely related receptor-like kinase genes ARK1, ARK2, and ARK3 sequences from several Brassicaceae taxa. The coding sequence of AlLal2 (NCBI gene ID 9305017) was included, the A. lyrata gene showing apparent orthology to LaLal2 as based on sequence similarity and conserved synteny (see above). Sequences homologous to Lal2 were identified in Capsella rubella (Carubv10025960m) and Brassica rapa (Bra010990). This was done as follows. First, pairwise alignments were generated between A. lyrata and L. alabamica, C. rubella, and Brassica rapa genomes, using lastz Harris (2007) in gapped, gfextend mode. These alignments were then chained Kuhn et al. (2012) to generate extended sets of alignments split by gaps of less than 100K Base. Low scoring chains were rejected and a subset of the highest scoring chains were annotated as candidate orthologous alignments between pairs of genomes. For the L. alabamica and B. rapa genomes, up to three orthologous chains were permitted for each region of the A. lyrata genome to represent orthology between the diploid and hexaploid contexts. The remaining chains were annotated as candidate homologous alignments. These alignment chains were used to identify candidate orthologs and homologs. The AlLal2 (NCBI gene ID 9305017), Carubv10025960, and Bra010990 predicted coding sequences were edited by sequence alignment of their genomic sequences with the Leavenworthia and A. lyrata Lal2 cDNA sequences obtained by sequencing. The outgroup for the analysis was selected from the sequences on the basis of closeness in evolutionary distance to the ingroup sequences as suggested by Lyons-Weiler et al. (1998), from the Brassicaceae family RLK sequences examined in Zhang et al. (2011).

The sequences were aligned using the default settings in Clustal Omega v. 1.1.0 Sievers et al. (2011) and the best-fit nucleotide substitution model for the alignment was determined by the Aikake Information Criterion as implemented in jModeltest v.0.1.1 [83,84]. MrBayes v. 3.1.2 Huelsenbeck et al. (2001) was used to carry out Bayesian phylogenetic inference under the GTR+I+┐ substitution model. All parameters were estimated during two independent runs of six Markov Monte Carlo chains, both of which were run for 4,000,000 generations (longer runs gave identical results). Phylogenetic trees were sampled every 4000^{th} generation and a consensus phylogeny was built from the 751 trees remaining after the first 250 were discarded as burn-in.

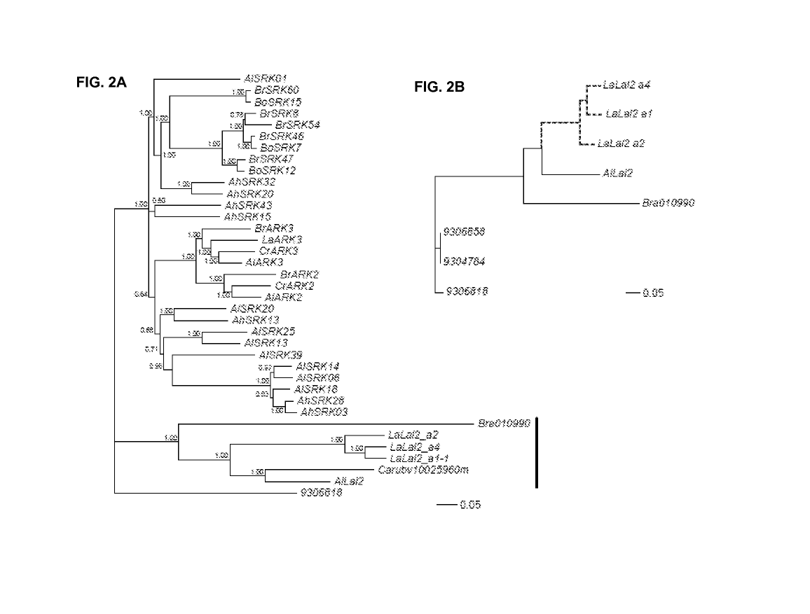

The branch-site model test for positive selection Zhang et al. (2005) at codon sites was carried out using the CODEML program in the PAML 4.4 package Yang (2007). The tree (FIG. 2B) was obtained using the PHYML Guindon et al. (2003) with default settings as implemented in Geneious v. 5.4.6 Drummond et al. (2011). Foreground branches for the branch-site model were assumed to be those in which LaLal2 evolved separately from related sequences in FIG. 2B.

Analysis of Synonymous and Non-Synonymous Substitution.

To determine whether sequence evolution of Lal2 associated with S locus evolution in this group was concentrated into particular protein domains, the sequence of the a1-1 haplotype was compared with that of the phylogenetically closest SRK sequence (allele SRK15 from Arabidopsis halleri). Estimates of synonymous and non-synonymous substitution and their ratios were obtained by maximum likelihood using the program CODEML in the PAML package Yang (2007). Estimated parameters for each major protein domain were compared by constraining them to be equal and carrying out the log likelihood ratio test.

Polymorphism Analysis of AlLal2 and AlSCR.

We amplified portions of AlLal2 and AlSCR from 10 individuals from the IND population of A. lyrata. Polymorphism data of genes unlinked to the S locus were obtained from Haudry et al. (2012). PCR primers are reported in Table 1 and PCR reaction protocols were identical to those reported above for RT-PCR. Amplicons were run on single-strand conformational polymorphism (SSCP) gels, as described in Herman et al. (2012), Busch et al. (2010). Bands corresponding to single-stranded products of AlLal2 and AlSCRL were cut from the gel, re-amplified and sent for Sanger sequencing at the McGill University and Génome Québec Innovation Centre (Montreal, Canada). Sequence trace files were edited by eye in Geneious v. 5.4.6 [75] and aligned to the reference copies of AlLal2 and AlSCRL, to which they were found to be identical.

Fosmid and PCR Cloning of the Lal2 Region in Different Races of Leavenworthia alabamica.

Leavenworthia alabamica includes several races that differ in floral characteristics and mating system. The L. alabamica populations studied here belong to three races. The a1 race consists of SI plants with large, strongly scented flowers, and outwardly dehiscing anthers. Plants of race a2 are SC, with large but weakly scented flowers, and partially inward dehiscing anthers, while a4 plants are also SC, but with small flowers lacking scent, and fully inward dehiscing anthers.

To better characterize the Leavenworthia alabamica Lal2 (LaLal2) gene and gain knowledge about its genomic context, fosmid libraries were constructed from single individuals of all three races. Clones containing LaLal2 were isolated after screening the libraries by PCR, and their sequences were obtained using 454 sequencing technology. The a1 race plant was heterozygous at LaLal2, whereas the a2 and a4 race plants were each homozygous for different LaLal2 alleles (whose S-domain sequences match those previously reported in these races). One LaLal2-containing clone was obtained from each of the a1 race and a2 race libraries (35,750 bp and 39,236 bp, respectively). From the a4 race library, two overlapping LaLal2 clones were isolated; these assembled into one long contig of 64,895 bp. The assembled sequences from the different L. alabamica races cover a similar genomic region, and they share a number of structural features characteristic of other Brassicaceae SRK/SCR S loci. They are referred below as Leavenworthia S haplotypes. Also included in our analysis are partial sequences, obtained by PCR amplification, of an additional S haplotype found in a population of fully SI plants belonging to the a1 race. This S haplotype contains a LaLal2 S-domain sequence identical to that of the SC race a2. To distinguish between the a1 haplotype from the a1 fosmid clone and this second a1 haplotype, they are referred to below as a1-1 and a1-2, respectively.

The Leavenworthia alabamica Lal2 Gene Encodes a Putative Receptor Kinase that Shares Highest Homology with a Paralog of SRK in A. lyrata.

Previous sequence information available for LaLal2 was limited to the portion of the sequence corresponding to the extracellular domain of members of the S-domain 1 (SD-1) receptor-like kinase (RLK) gene family to which SRK belongs. Analysis of the fosmid clones sequences allowed the full-length genomic sequence of LaLal2 to be determined. Homology of the full-length genomic LaLal2 sequence extends over the entire length expected for genes belonging to the SD-1 receptor kinase family. After excluding other Leavenworthia sequences, the highest match obtained from our BLASTn searches with the genomic LaLal2 sequence was NCBI Gene ID 9305017 from Arabidopsis lyrata (coverage 41%, E value 2e-10⁶), which has no characterized function (Table 2). For brevity the NCBI Gene ID 9305017 will be referred to as Arabidopsis lyrata Lal2 (AlLal2) gene. Other, lower similarity matches were to Brassicaceae SRK sequences. The LaLal2 coding regions were determined by combining data obtained from RT-PCR and 5′/3′ RACE sequences, which show that the gene has seven exons (FIG. 10A), as observed in SRK.

TABLE 2
Highest matches obtained in BLASTn searches using the full-length genomic sequence of the a1-1 Lal2 allele. Results were obtained
in June 2012 using the a1-1 LaLal2 full-length genomic as a query in searches performed in the NCBI nucleotide collection (nr/nt) database with
Leavenworthia sequences excluded in the search parameters.

| | | Max | Total | Query | | Max |
|---|---|---|---|---|---|---|
| Accession | Description | score | score | coverage | E value | ident |
| XM_002868900.1 | Arabidopsis lyrata subsp. lyrata predicted protein, mRNA | 398 | 544 | 41% | ###### | 75% |
| (NCBI Gene | | | | | | |
| ID_9305017) | | | | | | |
| XM_002866851.1 | Arabidopsis lyrata subsp. lyrata predicted protein, mRNA | 159 | 224 | 9% | 9.00E−35 | 80% |
| FJ670494.1 | Brassica cretica haplotype Bcr204c SRK protein gene, exons 4 through 7 and | 143 | 215 | 7% | 7.00E−30 | 84% |
| | partial cds | | | | | |
| FJ670493.1 | Brassica cretica haplotype Bcr204b SRK protein gene, exons 4 through 7 and | 143 | 215 | 7% | 7.00E−30 | 84% |
| | partial cds | | | | | |
| FJ670492.1 | Brassica cretica haplotype Bcr204a SRK protein gene, exons 4 through 7 and | 143 | 215 | 7% | 7.00E−30 | 84% |
| | partial cds | | | | | |
| FJ670491.1 | Brassica cretica haplotype Bcr203d SRK protein gene, exons 4 through 7 and | 143 | 215 | 7% | 7.00E−30 | 84% |
| | partial cds | | | | | |
| FJ670490.1 | Brassica cretica haplotype Bcr203c SRK protein gene, exons 4 through 7 and | 143 | 263 | 11% | 7.00E−30 | 84% |
| | partial cds | | | | | |
| FJ670489.1 | Brassica cretica haplotype Bcr203b SRK protein gene, exons 4 through 7 and | 143 | 215 | 7% | 7.00E−30 | 84% |
| | partial cds | | | | | |
| FJ670488.1 | Brassica cretica haplotype Bcr203a SRK protein gene, exons 4 through 7 and | 143 | 263 | 11% | 7.00E−30 | 84% |
| | partial cds | | | | | |
| FJ670485.1 | Brassica cretica haplotype Bcr201b SRK protein gene, exons 4 through 7 and | 143 | 211 | 7% | 7.00E−30 | 82% |
| | partial cds | | | | | |
| FJ670484.1 | Brassica cretica haplotype Bcr201a SRK protein gene, exons 4 through 7 and | 143 | 263 | 11% | 7.00E−30 | 82% |
| | partial cds | | | | | |
| AB298880.1 | Brassica rapa SRK-40 mRNA for S-locus receptor kinase, partial cds | 143 | 320 | 14% | 7.00E−30 | 84% |
| AB211197.1 | Brassica rapa SRK40 mRNA for S-receptor kinase, complete cds | 143 | 456 | 33% | 7.00E−30 | 84% |
| AB024416.1 | Brassica oleracea SRK2-b mRNA, complete cds | 143 | 445 | 23% | 7.00E−30 | 84% |
| EU075136.1 | Arabidopsis halleri S-receptor kinase (SRK) gene, SRK-AhSRK15 allele, exon | 141 | 141 | 6% | 2.00E−29 | 76% |
| | 1 and partial cds | | | | | |
| HQ379631.1 | Arabidopsis lyrata haplotype Aly-S50 S-locus region genomic sequence | 138 | 897 | 33% | 3.00E−28 | 77% |
| GQ351355.1 | Arabidopsis lyrata S-locus receptor kinase 25 (SRK25) gene, complete cds | 138 | 450 | 27% | 3.00E−28 | 87% |
| FJ670497.1 | Brassica cretica haplotype Bcr206b SRK protein gene, exons 4 through 7 and | 138 | 209 | 7% | 3.00E−28 | 84% |
| | partial cds | | | | | |
| FJ670496.1 | Brassica cretica haplotype Bcr206a SRK protein gene, exons 4 through 7 and | 138 | 209 | 7% | 3.00E−28 | 84% |
| | partial cds | | | | | |
| FJ670495.1 | Brassica cretica haplotype Bcr205 SRK protein gene, exons 4 through 7 and | 138 | 209 | 7% | 3.00E−28 | 84% |
| | partial cds | | | | | |
| FJ670487.1 | Brassica cretica haplotype Bcr202b SRK protein gene, exons 4 through 6 and | 138 | 209 | 7% | 3.00E−28 | 84% |
| | partial cds | | | | | |
| FJ670486.1 | Brassica cretica haplotype Bcr202a SRK protein gene, exons 5 through 7 and | 138 | 258 | 11% | 3.00E−28 | 84% |
| | partial cds | | | | | |
| AB298882.1 | Brassica rapa SRK-44 mRNA for S-locus receptor kinase (kinase domain), | 138 | 322 | 14% | 3.00E−28 | 84% |
| | partial cds | | | | | |
| AB270772.1 | Brassica napus BnSRK-6 gene for S receptor kinase, complete cds | 138 | 445 | 28% | 3.00E−28 | 84% |
| AB270768.1 | Brassica napus BnSRK-6 mRNA for S receptor kinase, partial cds | 138 | 435 | 28% | 3.00E−28 | 84% |
| AB180903.1 | Brassica oleracea S-15 SRK gene for S-locus receptor kinase, complete cds | 138 | 445 | 28% | 3.00E−28 | 84% |
| AB211198.1 | Brassica rapa SRK44 mRNA for S-receptor kinase, complete cds | 138 | 395 | 24% | 3.00E−28 | 84% |
| Y18260.1 | Brassica oleracea mRNA for SRK15 protein, partial | 138 | 435 | 28% | 3.00E−28 | 84% |
| Y18259.1 | Brassica oleracea mRNA for SRK5 protein, partial | 138 | 442 | 30% | 3.00E−28 | 84% |

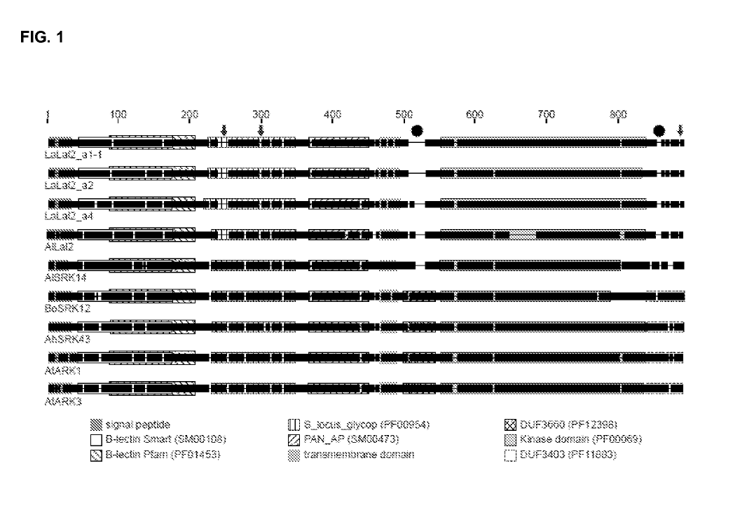

The predicted amino acid sequences of LaLal2 and AlLal2 have signal peptide and transmembrane domain signature sequences, as expected for a transmembrane receptor coding sequence (FIGS. 1 and 10B). Domain organization of LaLal2 and AlLal2 proteins predicted by the SMART/Pfam online program Letunic et al. (2011) is as follows: two overlapping B-Lectin domains, an S_locus_glycoprotein domain and a PAN_APPLE domain in their extracellular domain, and an intracellular catalytic kinase domain, the latter being made up of the eleven subdomains described for protein kinases (FIGS. 1 and 10B). In addition to these domains, most of the known SRK alleles as well as their most closely related SD-1 RLK gene family members, ARK1 and ARK3, also possess DUF3660 and DUF3403 domains (FIG. 1). Alignment of amino acid sequences of LaLal2 and AlLal2 to those of Brassicaceae SRK alleles (e.g. AlSRK14, BoSRK12, and AhSRK43) as well as to those of A. thaliana ARK1 and ARK3 produced gaps in Lal2 sequences in regions corresponding to the DUF3660 and DUF3403 domains. Although A. lyrata and A. halleri SRK sequences belonging to the class B SRK alleles also lack these two predicted domains (e.g. AlSRK14 and AhSRK28) their sequences cluster phylogenetically within the clade of SRK alleles and not with the Lal2 sequences (FIGS. 1, 11 and 2). Moreover, upon closer examination of the regions around the deletions of DUF3660 and DUF3403 in class B SRK alleles (around residues 535 and 870, respectively), the amino acid residues flanking the deletions are seen to be more similar to SRK and ARK then to Lal2 (FIG. 11). There are also a number of alignment gaps that were found to be specific to all LaLal2 and AlLal2 sequences (FIGS. 1 and 11). Altogether, LaLal2 and AlLal2 appear to be gene orthologs that code for a type of SD-1 receptor kinase that is closely related to but distinct from SRK sequences.

Phylogenetic Analyses of the Leavenworthia Lal2 Gene and Related Sequences.

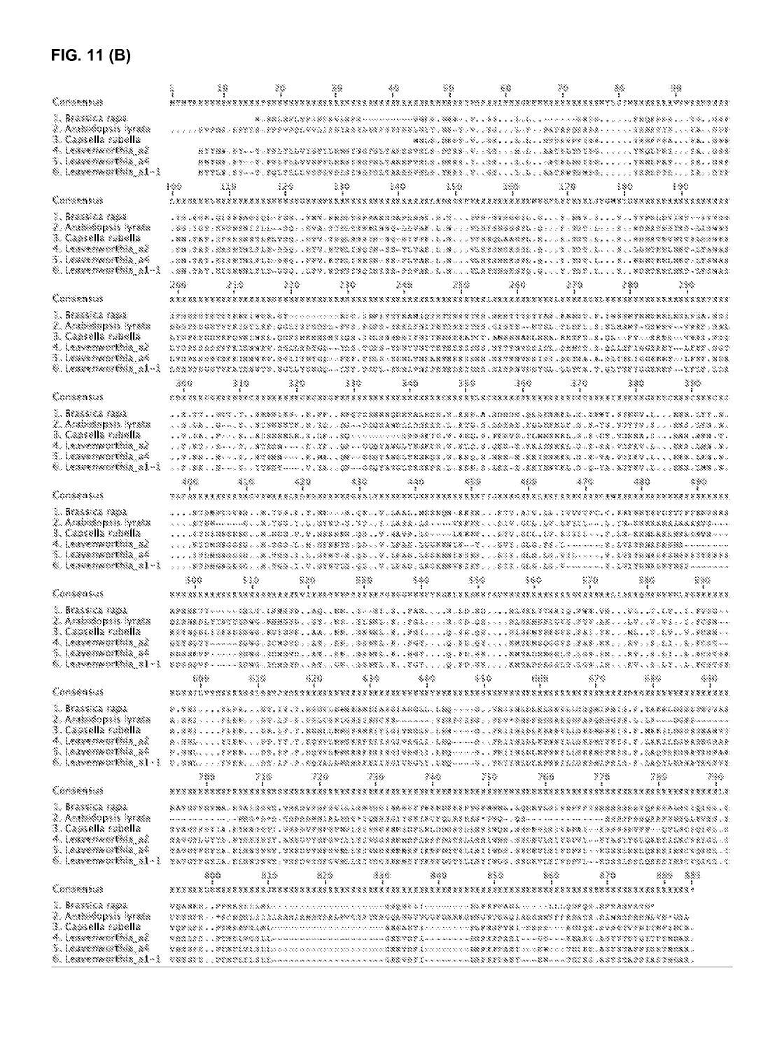

Lal2-like sequences were found in Brassica rapa (Bra010990) and Capsella rubella (Carubv10025960), though in genomic regions not syntenic with Leavenworthia and A. lyrata Lal2. Phylogenetic analysis of the full-length coding sequence of LaLal2 alleles, AlLal2, and these Lal2-like sequences from C. rubella, and B. rapa, together with that of SRK and the SRK-related sequences (e.g., ARK2 and ARK3) of other Brassicaceae species showed that the Lal2 group and the SRK-ARK group form two separate clades which appear to have diverged before the onset of the strong allelic diversification of SRK (FIG. 2A). Lal2-like sequences from C. rubella, and B. rapa also form part of this clade, and show the topological relationship in the tree expected from species relationships, as do the ARK3 sequences within the SRK-ARK clade. Similar results were obtained when phylogenetic analysis is based only on the S-domain portion of the sequence, or on the transmembrane and kinase domain portions (FIGS. 12A and 12B), which suggests that the phylogenetic pattern of separate diversification of Lal2 is unlikely to be due to a domain-swapping event that may have modified a hypothetical duplicate of SRK. Synonymous and non-synonymous substitutions differentiating LaLal2 and SRK sequences do not appear to be concentrated in any one portion of the gene (Table 3).

TABLE 3
Estimates of the ratio and rates of non-synonymous and synonymous
substitution per site for four major protein domains in a comparison of Lal2
and SRK coding sequences. Sequences compared are LaLal2 (a1-1
haplotype) and Arabidopsis halleri SRK15. Maximum likelihood estimates
of parameters obtained using the PAML package program CODEML Yang
(2007). In the matrix portion of the table (below the estimates), the upper
diagonal gives the log likelihood ratio test statistic value when dN/dS ratios
are constrained to be equal for the comparison denoted in each cell. The
lower diagonal gives the absolute value of the difference between the dN/dS
ratios for the comparison denoted in each cell. The test statistic is distributed
as Chi square with 1 degree of freedom. None of the pairwise comparisons
are statistically significant.

| Domain | dN/dS | dN | dS | Nucleotides |
|---|---|---|---|---|
| B-lectin | 0.3293 | 0.3726 | 1.1312 | 450 |
| S_locus_glycoprotein | 0.2581 | 0.6138 | 2.3781 | 291 |
| Pan_Apple | 0.2062 | 0.2413 | 1.1701 | 240 |
| Kinase | 0.2106 | 0.3368 | 1.5989 | 849 |
| | | S-locus | | |
| Domain | B-lectin | glycoprotein | Pan-Apple | Kinase |
| B-lectin | — | 0.9724 | 3.3746 | 3.0970 |
| S_locus_glycoprotein | 0.07129 | — | 0.2293 | 0.1911 |
| Pan_Apple | 0.12317 | 0.0518 | — | 0.0032 |
| Kinase | 0.11875 | 0.0475 | 0.0044 | — |

The branch-site model test Zhang et al. 2005) was applied to detect positive selection at individual codon sites in LaLal2 sequences following their divergence from the most closely related sequences in the phylogeny (FIG. 2B). The test rejects the null hypothesis of no selection, and indicates that at least one codon (located in the hypervariable region of the S-domain described in Busch et al. (2008)) has undergone positive selection (Likelihood ratio test statistic=8.426, P<0.005) following divergence from the other sequences.

A Defensin-Like Encoding Gene is Located in the Genomic Vicinity of LaLal2.

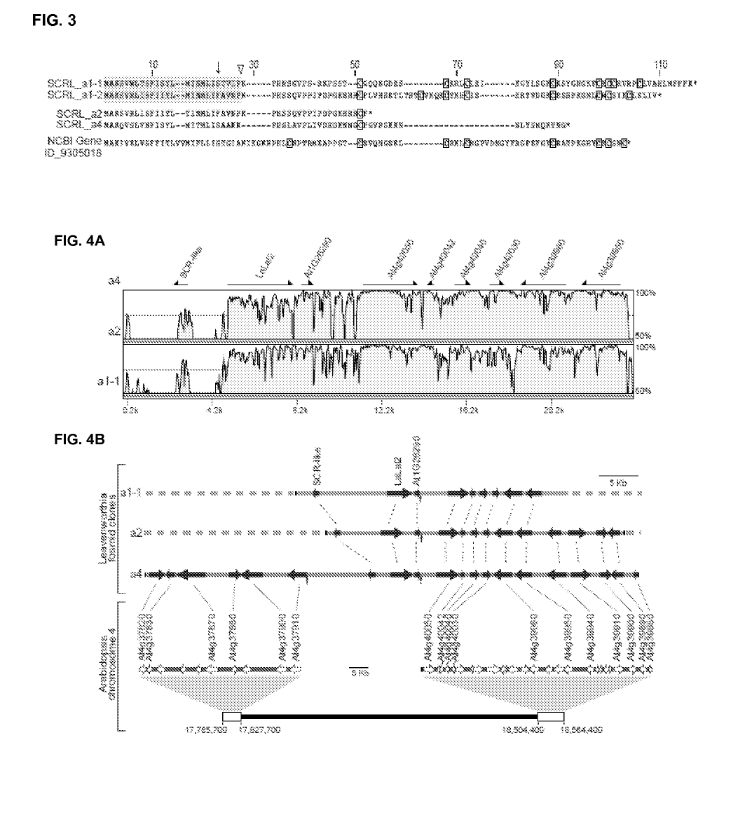

It has been noted that the SCR gene in previously characterized Brassicaceae S-locus haplotypes has the structure of a plant defensin. In the three fosmid clones sequenced, a gene exhibiting characteristics of a plant defensin was found ca. 2 000-10 000 bp upstream of LaLal2. This gene is referred to below as SCR-like (SCRL). The LaSCRL alleles of the a1-1 and a1-2 haplotypes contain full open reading frames and were used for further sequence analysis of the gene. Based on their cDNA sequences, it was established that the SCRL gene consists of two exons, a characteristic common to the majority of plant defensin encoding genes. Analysis with the SignalP online tool predicts that the coding sequences of a1-1 and a1-2 LaSCRL translate into preproteins composed of an N-terminal signal peptide, required for protein secretion, and a small hydrophilic mature protein (FIG. 3). The cleavage site of the signal peptide is predicted to be located after amino acid 25 in both a1-1 and a1-2 LaSCRL, generating mature proteins of 67 amino acids (aa) and 70 aa respectively. While the signal peptide sequences of a1-1 and a1-2 LaSCRL are partially conserved (72% aa identity), the mature protein sequences are highly variable (32% identity), though like SCR, they contain eight cysteine residues (although their positions are not well conserved in the two sequences). Protein structure prediction using the modeling packages I-TASSER and DiANNA suggests that the LaSCRL product has a compact tertiary structure formed by disulfide bridges between a number of the cysteine residues, as seen in the SCR's of other Brassicaceae.

BLAST searches with the cDNA sequence or the amino acid sequence of a1-1 LaSCRL found only a limited number of significant hits. As with LaLal2, however, the genes with highest similarity are found in A. lyrata: genes NCBI Gene ID 9302985 and NCBI Gene ID 9305018 (Table 4), neither of which has known functions. Sequence similarity with the two A. lyrata genes is mainly restricted to exon 1 of SCRL, which corresponds to most of the signal peptide sequence. NCBI Gene ID 9302985 and NCBI Gene ID 9305018 (FIG. 3) are predicted to also encode mature proteins containing eight cysteine residues and that show low sequence identity with LaSCRL. Phylogenetic analysis was not possible with SCRL and SCR sequences due to difficulties in aligning the regions.

TABLE 4A
Highest matches obtained in BLASTn searches using the cDNA sequences of the a1-1 SCRL allele.

| | | Max | Total | Query | | Max |
|---|---|---|---|---|---|---|
| Accession | Description | score | score | coverage | E value | ident |
| XM_002866867.1 | Arabidopsis lyrata subsp. lyrata hypothetical protein, mRNA | 66.2 | 66.2 | 22% | 1.00E−07 | 83% |
| (NCBI Gene | | | | | | |
| ID_9302985) | | | | | | |
| CP001560.1 | Escherichia blattae DSM 4481, complete genome | 46.4 | 46.4 | 17% | 1.00E−01 | 82% |
| AC120985.3 | Oryza sativa Japonica Group chromosome 5 clone OJ1532_D06, | 44.6 | 44.6 | 11% | 3.50E−01 | 91% |
| | complete sequence | | | | | |
| JN730534.1 | Cyprinus carpio clone 292821 microsatellite sequence | 41 | 41 | 11% | ##### | 88% |
| HE601624.1 | Schistosoma mansoni strain Puerto Rico chromosome 1, complete | 41 | 41 | 8% | ##### | 96% |
| | genome | | | | | |
| HQ664953.1 | Human parvovirus B19 strain DRK1 NS1 gene, partial cds; and | 41 | 41 | 9% | ##### | 93% |
| | VP1/2 gene, complete cds | | | | | |

TABLE 4B
Highest matches obtained in BLASTn searches using the the amino acid sequences of the a1-1 SCRL allele.

| | | Max | Total | Query | | Max |
|---|---|---|---|---|---|---|
| Accession | Description | score | score | coverage | E value | ident |
| XP_002866915.1 | hypothetical protein ARALYDRAFT_912515 [Arabidopsis lyrata | 47 | 47 | 82% | 2.00E−05 | 38% |
| (NCBI Gene | subsp. lyrata] >gb\|EFH43174.1\|hypothetical protein | | | | | |
| ID_9305018) | ARALYDRAFT_912515 [Arabidopsis lyrata subsp. lyrata] | | | | | |
| XP_002866913.1 | hypothetical protein ARALYDRAFT_912510 [Arabidopsis lyrata | 44.3 | 44.3 | 82% | 2.00E−04 | 33% |
| (NCBI Gene | subsp. lyrata] >gb\|EFH43172.1\|hypothetical protein | | | | | |
| ID_9302985) | ARALYDRAFT_912510 [Arabidopsis lyrata subsp. lyrata] | | | | | |
| XP_002878772.1 | hypothetical protein ARALYDRAFT_320269 [Arabidopsis lyrata | 35.8 | 35.8 | 70% | 0.18 | 36% |
| | subsp. lyrata] >gb\|EFH55031.1\|hypothetical protein | | | | | |
| | ARALYDRAFT_320269 [Arabidopsis lyrata subsp. lyrata] | | | | | |
| P0CAY1.1 | RecName: Full = Putative defensin-like protein 42 | 35.4 | 35.4 | 64% | 0.2 | 36% |
| NP_001031408.1 | putative defensin-like protein 38 [Arabidopsis thaliana] | 35.4 | 35.4 | 70% | 0.22 | 38% |
| | >sp\|Q2V462.1\|DEF38_ARATH RecName: Full = Putative defensin- | | | | | |
| | like protein 38; Flags: Precursor >gb\|AEC07602.1\|putative | | | | | |
| | defensin-like protein 38 [Arabidopsis thaliana] | | | | | |
| XP_002880279.1 | hypothetical protein ARALYDRAFT_904180 [Arabidopsis lyrata | 34.7 | 34.7 | 72% | 0.47 | 32% |
| | subsp. lyrata] >gb\|EFH56538.1\|hypothetical protein | | | | | |
| | ARALYDRAFT_904180 [Arabidopsis lyrata subsp. lyrata] | | | | | |
| AAT92145.1 | putative salivary secreted peptide [Ixodes pacificus] | 34.7 | 34.7 | 84% | 0.5 | 28% |
| XP_002376253.1 | conserved hypothetical protein [Aspergillus flavus NRRL3357] | 35.8 | 35.8 | 86% | 0.56 | 25% |
| | >ref\|XP_003190084.1\|hypothetical protein AOR_1_1742194 | | | | | |
| | [Aspergillus oryzae RIB40] >gb\|EED54981.1\|conserved | | | | | |
| | hypothetical protein [Aspergillus flavus NRRL3357] | | | | | |
| YP_004347099.1 | hypothetical protein LAU_0136 [Lausannevirus] >gb\|AEA06987.1\| | 33.9 | 33.9 | 86% | 2.9 | 29% |
| | hypothetical protein LAU_0136 [Lausannevirus] | | | | | |
| NP_001030645.1 | defensin-like protein 204 [Arabidopsis thaliana] | 32.3 | 32.3 | 82% | 3 | 29% |
| | >sp\|Q56XB0.1\|DF204_ARATH RecName: Full = Defensin-like | | | | | |
| | protein 204; Flags: Precursor >dbj\|BAD93850.1\|hypothetical protein | | | | | |
| | [Arabidopsis thaliana] >gb\|AEE74286.1\|defensin-like protein 204 | | | | | |
| | [Arabidopsis thaliana] | | | | | |
| YP_004093464.1 | signal peptidase I [Bacillus cellulosilyticus DSM 2522] | 33.1 | 33.1 | 70% | 4.8 | 26% |
| | >gb\|ADU28733.1\|signal peptidase I [Bacillus cellulosilyticus DSM | | | | | |
| | 2522] | | | | | |
| XP_002196497.1 | PREDICTED: tubulin, delta 1 [Taeniopygia guttata] | 32.7 | 32.7 | 43% | 8.4 | 40% |
| NP_001031400.1 | putative defensin-like protein 191 [Arabidopsis thaliana] | 31.2 | 31.2 | 82% | 8.6 | 30% |
| | >sp\|Q2V466.1\|DF191_ARATH RecName: Full = Putative defensin- | | | | | |
| | like protein 191; Flags: Precursor >gb\|AEC07378.1\|putative | | | | | |
| | defensin-like protein 191 [Arabidopsis thaliana] | | | | | |

A Syntenic Genomic Block of Arabidopsis lyrata on Chromosome 7 Contains Orthologs of LaLal2 and LaSCRL.

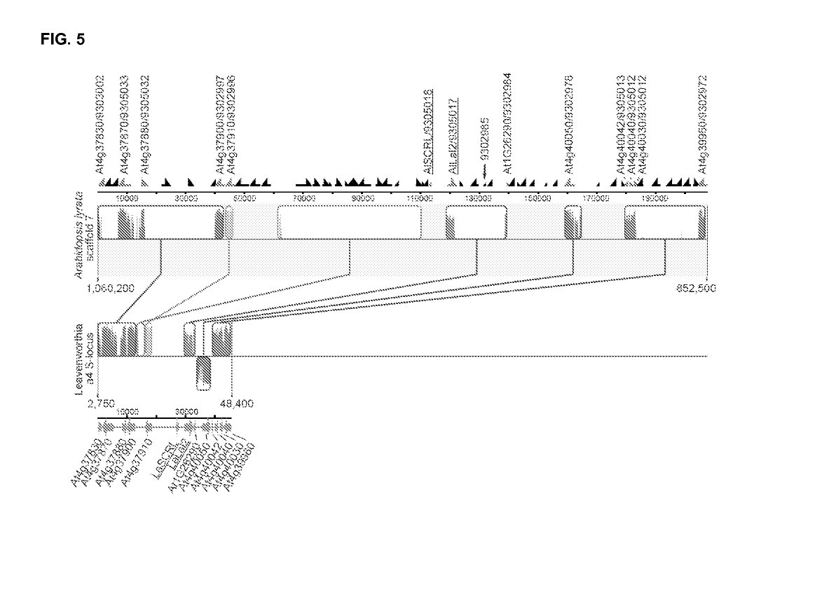

Alignment of the three fosmid sequences together with sequence similarity searches in the A. thaliana genome database revealed that the diversity pattern in this Leavenworthia genomic region resembles the SRK/SCR S-locus region of other characterized Brassicaceae species. The LaLal2 and LaSCRL genes themselves have high sequence diversity, but are flanked (at least on the right of LaLal2) by highly conserved regions (FIG. 4A). If the core S locus is defined as being the region of low sequence similarity between the three haplotypes and comprising LaLal2 and LaSCRL, the size of the S locus is 14 kb in the a4 haplotype, the only one for which sequence information on both sides of the S locus is available. Because the upstream sequences of the core S locus of the a1-1 and a2 haplotypes are currently undetermined, their sizes remain unknown, but are at least 15.3 kb in the a1-1 haplotype and 11.4 kb in the a2 haplotype. In all three Leavenworthia haplotypes, the LaLal2 and LaSCRL transcription units are arranged tail-to-tail and the gene order is the same.

Annotation of the fosmid sequences using the A. thaliana reference genome revealed that the conserved regions on each side of the Leavenworthia core S locus are syntenic with an A. thaliana chromosome 4 region (FIG. 4B). This region contains genes annotated as At4g37820 to At4g37910 on one side of the Leavenworthia core S locus, and genes At4g40050 to At4g39880 on the other side, but none with sequence homology to LaLal2 or LaSCRL. Moreover, there are no reports of an S locus in this region in other Brassicaceae species that have been examined to date, including A. lyrata.

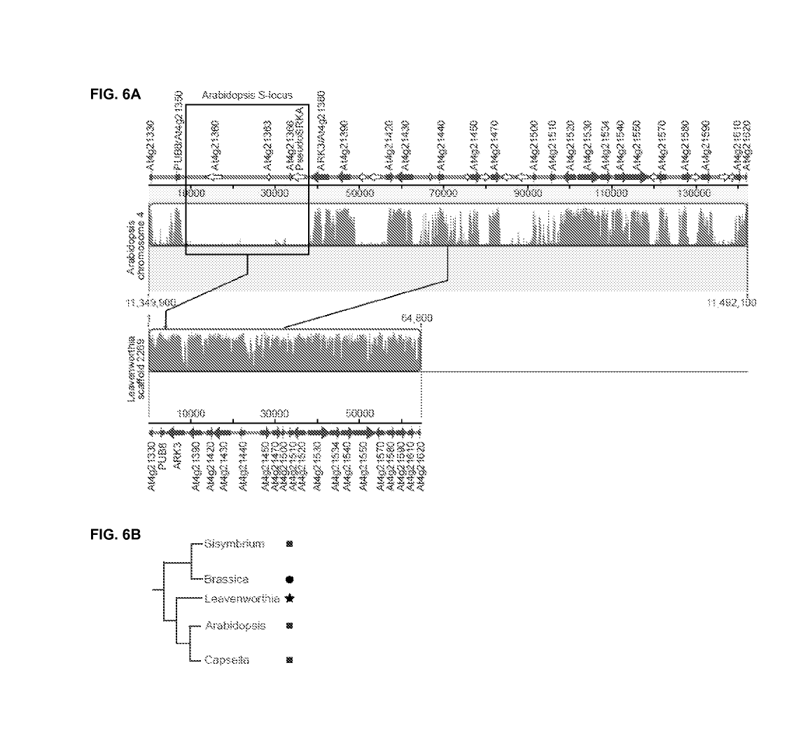

As noted above, however, LaLal2 and LaSCRL do show sequence homology to annotated but uncharacterized genes in A. lyrata, with highest homology to, respectively, NCBI Gene ID numbers 9305017 (called here AlLal2), and NCBI Gene ID numbers 9302985 and NCBI Gene ID numbers 9305018. All three genes are located in close proximity on A. lyrata scaffold 7 and, notably, AlLal2 and NCBI Gene ID 9305018 are positioned only 9.8 kb apart, and are in a tail-to-tail configuration, like LaLal2 and LaSCRL in Leavenworthia (FIG. 5). Below to the NCBI Gene ID 9305018 of A. lyrata is referred to as AlSCRL. Annotation of the surrounding genomic sequence using the A. thaliana reference genome revealed that this A. lyrata scaffold 7 region (between positions 852,500 bp and 1,060,200 bp) contains genes with annotations identical to all the genes found in the Leavenworthia a4 haplotype fosmid sequence. Most are homologous to genes on A. thaliana chromosome 4. However, a gene homologous to At1g26290 located on A. thaliana chromosome 1 was found in all three Leavenworthia haplotypes (between LaLal2 and the Leavenworthia At4g40050 homolog), as well as in the A. lyrata syntenic genomic region (FIGS. 4 and 5).

In addition to the region homologous to the Leavenworthia Lal2/SCRL S-locus region, A. lyrata chromosome 7 also carries the SRK/SCR S locus, the latter being located at positions 9,335,860 bp (NCBI gene ID 9303924/ARK3) to 9,377,892 bp (NCBI gene ID 9305963/PUB8). The A. thaliana region carrying the SRK/SCR S-locus orthologous genes is also located between genes At4g21350 (PUB8) and At4g21380 (ARK3), in the homologous chromosome 4 region. Although the A. lyrata region with the homologs of the Leavenworthia LaLal2 region genes is also on chromosome 7, it is more than 8 Mb away from the S-locus region.

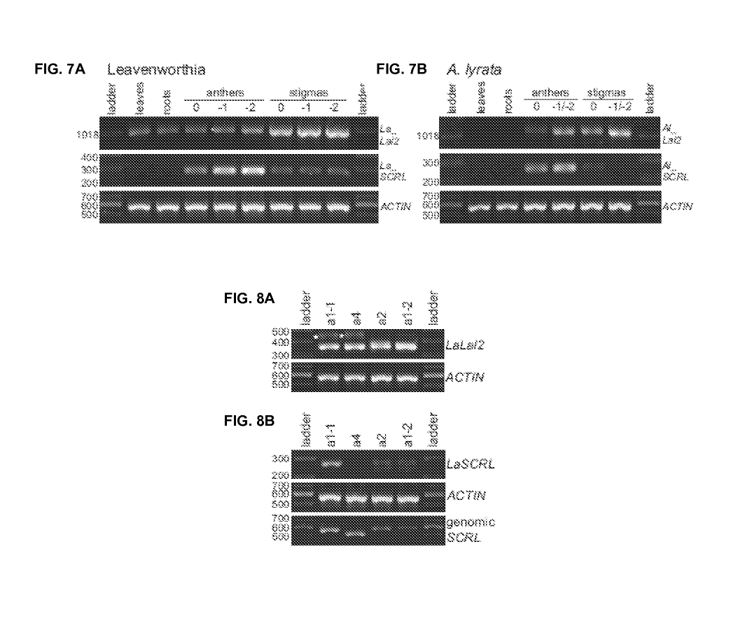

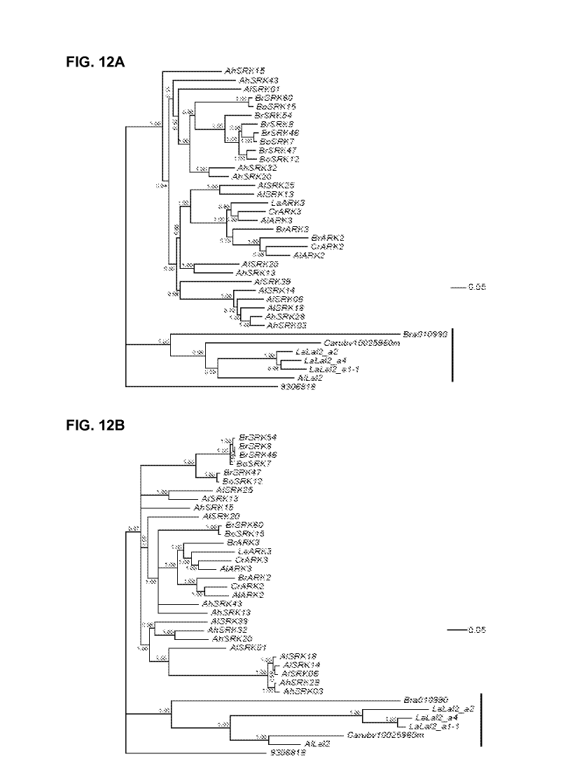

The syntenic Arabidopsis S-locus region in Leavenworthia does not contain SRK and SCR. Conversely, the Leavenworthia genomic region carrying the homologs of the Arabidopsis SRK/SCR S-locus genes were identified from data obtained in an ongoing project to sequence the Leavenworthia alabamica race a4 plant genome (http://biology.mcgill.ca/vegi/index.html). This Leavenworthia genomic scaffold is syntenic to genomic blocks found in the SRK/SCR S-locus region of A. thaliana (FIG. 6A). Of special interest is the observation that the genomic block located between PUB8 and ARK3, which contains the SRK and SCR genes in Arabidopsis species, is highly reduced in length in L. alabamica, which if of 1.1 kb from the stop codon of the ARK3 ortholog to the start codon of the PUB8 ortholog (versus 4231 kb in the shortest A. lyrata S locus sequenced to date), and neither SRK or SCR is present. PCR amplification and sequencing of the ARK3-PUB8 region in an a1-1 S haplotype homozygote plant confirmed the absence of SRK and SCR orthologs in that region in a SI individual as well (FIG. 13). This result is consistent with earlier crossing studies that showed that Lal8, the putative Leavenworthia ARK3 ortholog, does not co-segregate with SI reactions. Other PUB8 and ARK3 orthologs were not found in any other Leavenworthia genomic region.

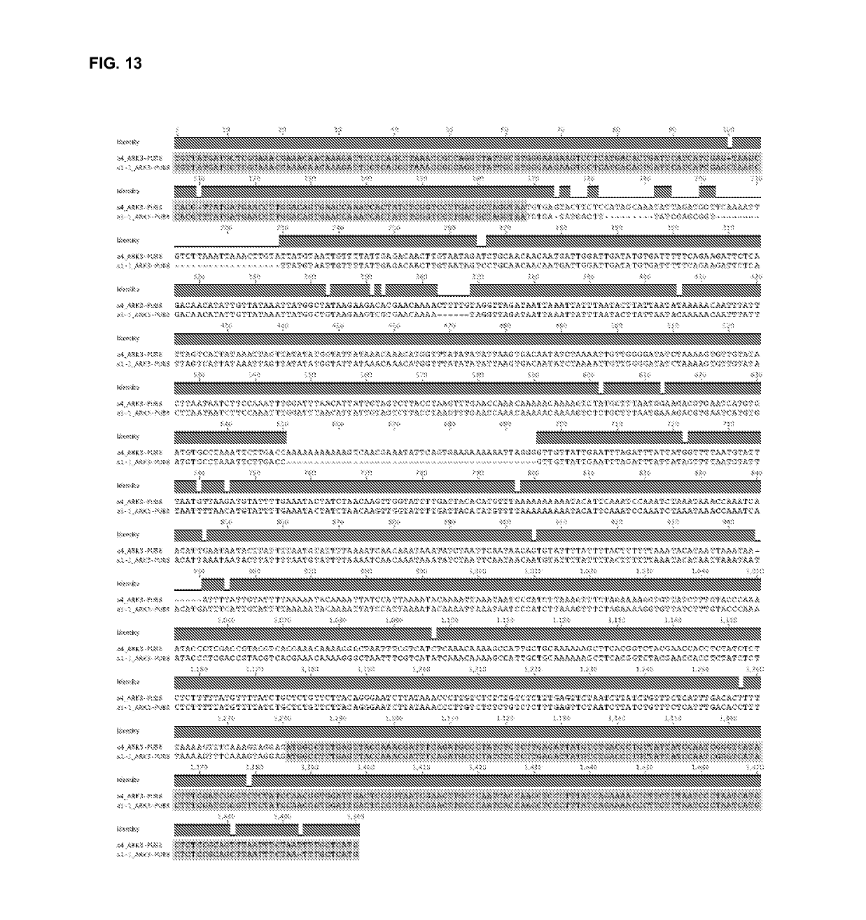

It is informative to compare S locus locations in different Brassicaceae species for which data are available. To date, S loci have been reported in 3 different synteny blocks. As part of the genome sequencing project mentioned above, it was determined that Sisymbrium irio has a putative SRK ortholog with an apparently intact open reading frame (despite the fact that this species is self-compatible), with a location similar to that of Arabidopsis SRK gene (FIG. 14). In Capsella rubella [42], the S locus also occupies a genomic region syntenic to the Arabidopsis SRK/SCR S locus (on scaffold 7, between positions 7,520,515 bp (Carubv10007030m/ARK3) and 7,563,814 bp (Carubv10005064m/PUB8)). In Brassica, the S locus genomic location is different, lying between orthologs of A. thaliana At1g66680 and At1g66690 (on chromosome 1 of Brassica rapa, between positions 17,225,424 bp (Bra004178/At1g66680) and 17,282,231 bp (Bra4183/At1g66690)). The S locus locations and phylogenetic relationships of these genera are summarized in FIG. 6B, which suggests that the Arabidopsis SRK/SCR S locus location is ancestral.

Expression Pattern Analysis of Lal2 and SCRL in Leavenworthia and A. lyrata.

Given the conservation of sequence and synteny described above for LaLal2 and LaSCRL versus AlLal2 and AlSCRL, an expression pattern study was conducted by RT-PCR of the two genes in a Leavenworthia plant homozygous for the a1-1 S haplotype and a A. lyrata SI individual in an effort to determine whether they could play a role in SI, or may have played such a role earlier in the evolutionary history of A. lyrata.

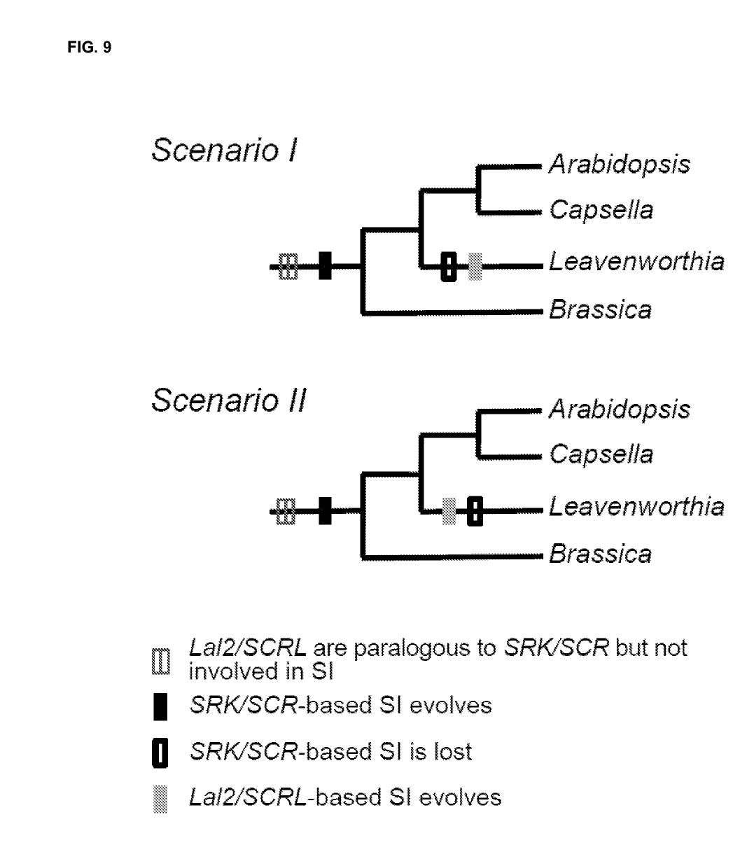

It was shown previously that the SRK gene is more highly expressed in stigmas and that the SCR gene is expressed in anthers in Brassica and Arabidopsis, which is concordant with their respective roles in the SI mechanism. In Leavenworthia, LaLal2 expression was detected at similar levels in leaves, roots, and anthers and at higher levels in stigmas at the different stages of flower development (FIG. 7A). In A. lyrata, AlLal2 expression was detected in anthers and stigmas at the different stages of flower development but not in leaves and roots (FIG. 7B). As for the SCRL gene, its expression in Leavenworthia was detected in anthers, most strongly two days or one day before anthesis, and at lower levels in anthers at flower opening (stage 0), and in stigmas at the different stages of flower development (FIG. 7A). LaSCRL expression could not be detected in leaves and roots. A similar expression pattern was observed for AlSCRL in A. lyrata (FIG. 7B). Although the expression of LaLal2 is not specific to stigmas and the expression of LaSCRL is not specific to anthers (was also found in stigmas, which was also shown for SCR/SP11 in Brassica when using RT-PCR), their expression in stigmas and in anthers, respectively, in higher levels than in other tissues is in accordance with their involvement in the SI mechanism.

To compare the relative expression levels of AlLal2 vs AlSRK and AlSCRL vs AlSCR in A. lyrata, the RNAseq data obtained from flower buds (stage 12) of the MN47 strain was also analyzed. The analysis indicated that AlLal2 exhibits less than 8% the expression level compared with that of AlSRK, and that AlSCRL exhibits less than 5% the expression level compared with that of AlSCR (Table 5).

TABLE 5
RNAseq expression analysis of AlLal2, AlSCRL, SRK and SCR in
Arabidopsis lyrata strain MN47. Cells values in table are in units of
fragments per kilobase of exon per million fragments mapped (FPKM).
Library sizes are as follows: root (34 × 10⁶ reads). flower bud
(25.3 × 10⁶ reads) and seedling (26.2 × 10⁶ reads).

| | AlLal2 | AlSCRL | SRK | SCR |
|---|---|---|---|---|
| root | 0 | 0 | 0 | 0 |
| flower bud | 0.292963612 | 28.98805663 | 3.820121805 | 580.0137928 |
| (stage 12) | | | | |
| seedling | 0 | 0 | 0.214032795 | 0 |

Polymorphism Analysis of AlLal2 and AlSCRL.

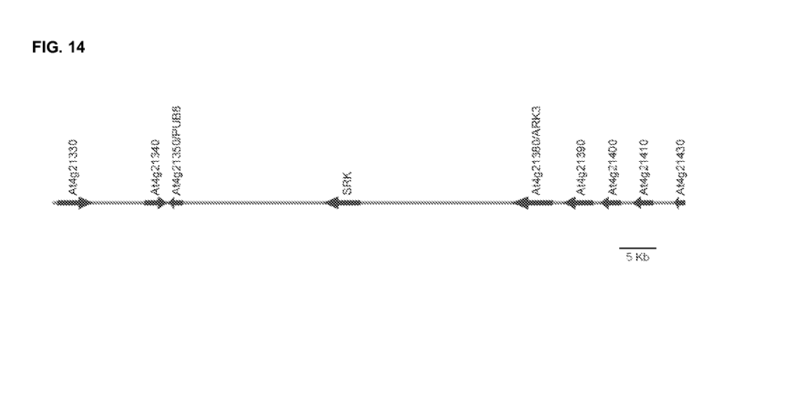

It was examined whether the A. lyrata Lal2 and SCRL genes exhibit a pattern of high polymorphism that would be expected if they play a role in SI. The S-domain of AlLal2 was amplified and the majority of the sequence of AlSCRL from 10 individuals in a single SI population (Population IND) located in Indiana. PCR products were visualized on SSCP gels. Banding patterns across 10 individuals were identical for both genes, suggesting monomorphism in the population (FIG. 15). The single-stranded products were sequenced for each gene and these results show the presence of only one allele at each locus. This is in contrast to the observed high levels of polymorphism exhibited in the same population where the synonymous polymorphism for genes unlinked to SRK is σ=0.013 suggesting that there is no evidence for a genome-wide population bottleneck in this population.

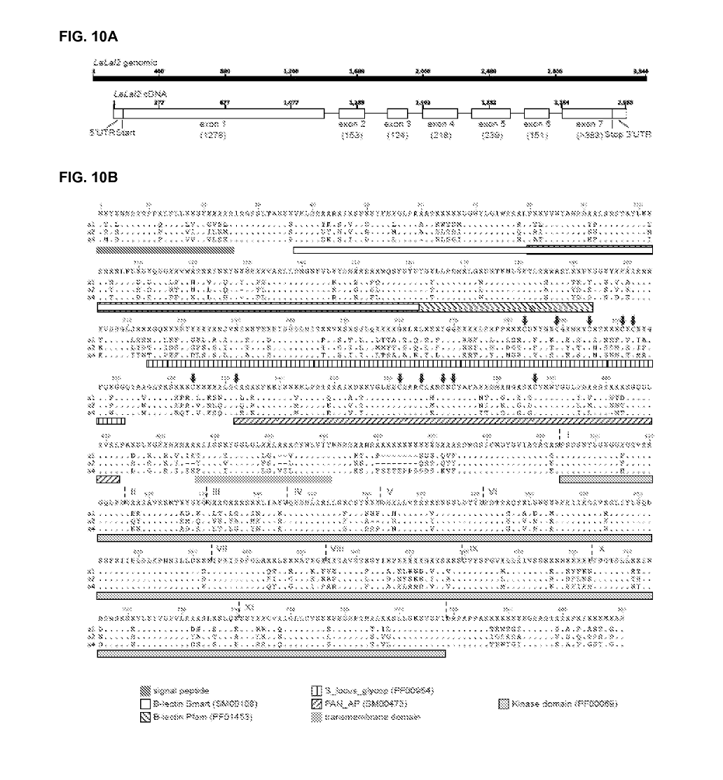

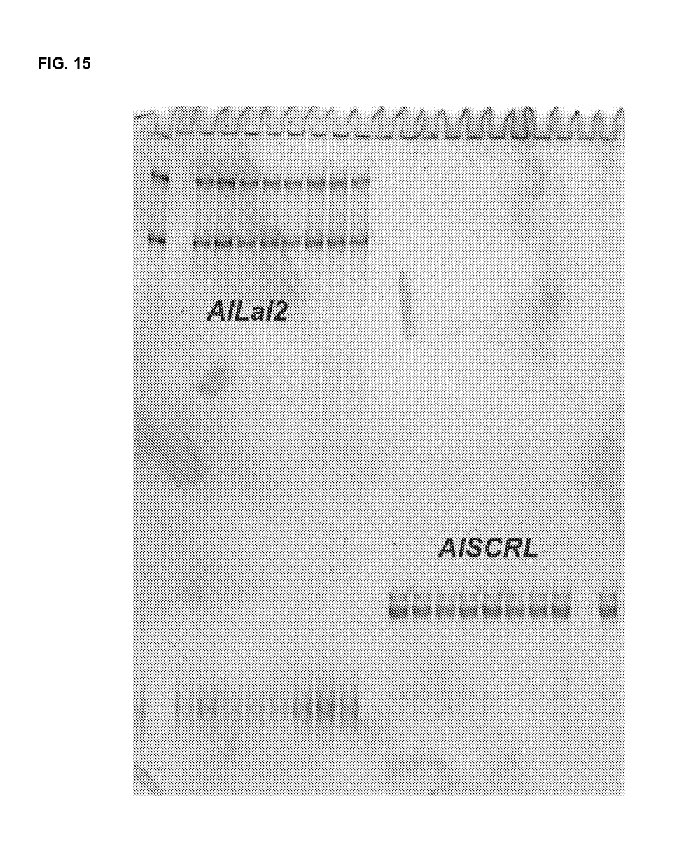

The SC races of Leavenworthia alabamica possess mutations in the SCR-like gene. The sequences of the a2 and a4 S haplotypes were obtained with the goal of determining the nature of loss of SI in these Leavenworthia SC races, particularly by analyzing sequences and expression of LaLal2 and LaSCRL in plants homozygous for the a1-1, a2 or a4 haplotypes. In these analyses the a1-2 haplotype found in SI plants of the a1 race was included. The a1-2 LaLal2 allele encodes an S-domain sequence identical to that of the a2 allele (FIG. 16), and these two alleles should therefore have the same SCRL pollen specificity. None of the LaLal2 allele sequences includes any mutations disrupting the coding sequence (FIG. 10B). Using stigmas of flower buds two days before anthesis, it was found that LaLal2 is expressed at similar levels in plants homozygous for each of the S-locus haplotypes described in this study (FIG. 8A).

In contrast, analysis of LaSCRL sequences and expression revealed that the a2 and a4 alleles, from SC races, have various disruptive mutations. In our race a4 plant, no LaSCRL expression could be detected in anthers two days before anthesis (FIG. 8B), a development stage at which the a1-1 LaSCRL allele is highly expressed (FIG. 7A). The coding region of the a4 LaSCRL allele deduced from the genomic DNA sequence contains a premature stop codon and the cleavage site of the signal peptide appears to be defective compared to that of the a1-1 and a1-2 LaSCRL alleles (FIG. 3). Expression of the a2 LaSCRL allele was detected in anthers two days before anthesis (FIG. 8B) but its translated sequence differs from that of a1-2 by one amino acid residue, and there is a premature stop codon after amino acid residue 45 (FIG. 3). Plants homozygous for the a1-2 haplotype or the a2 haplotype were crossed to determine whether their incompatibility reactions fit those expected based on the sequence differences outlined above. The plant with the a1-2 haplotype appears to be compatible as a pollen recipient when a2 plants are used as pollen donors (89% of 9 crosses produced fruit or had germinated pollen tubes). In contrast, the reciprocal crosses (a2 recipient plants and a1-2 pollen donors), appear to be incompatible with only 10% of 20 crosses that produced a fruit or had germinated pollen tubes. These proportions are significantly different (Z=4.135, P<0.001), and support the hypothesis that self-compatibility in the a2 race is due to a mutation in SCRL (a1-2 pollen was shown to produce offspring when used in crosses with other pollen recipients). These results suggest that, as in other Brassicaceae, Leavenworthia possesses an S locus, which when disrupted leads to self-compatibility. Loss of SI in Leavenworthia a2 and a4 races is probably not due to loss of LaLal2 function, but to mutations in the male function SCRL gene. It is not known whether putative downstream genes in the SI pathway (e.g., ARC1, MLPK) are functional or not in all race a4 plants, though ARC1 appears to be deleted in a plant obtained from one a4 race (self-compatible) population.

The S locus of Leavenworthia is unusual. The Leavenworthia S locus was characterized in detail and it comprises two closely linked genes located in a genomic region of low sequence conservation among Leavenworthia haplotypes, as is also the case for the SRK/SCR S locus in other Brassicaceae members. The two Leavenworthia S-locus genes, LaLal2 and LaSCRL, resemble the S-locus genes SRK and SCR in their sequence and expression pattern, and unlike their orthologs in populations of Arabidopsis lyrata, they are highly polymorphic. Phylogenetic trees constructed from Leavenworthia Lal2 alleles show a pattern of long terminal branches similar to that observed at SRK/SCR S loci.

While previous studies indicated the existence of a functional S locus in the SI Leavenworthia races, the results reported here suggest that the genes comprising the Leavenworthia Lal2/SCRL S locus are unlike those of other Brassicaceae S loci that have been characterized to date. First, in Leavenworthia, SRK and SCR are absent from the syntenic block in which they occur in Arabidopsis and its close relatives, a genomic position that appears to be ancestral in the Brassicaceae. This is true in the case of the Brassica S locus as well, where it has been suggested that translocation of the entire S locus may have occurred. However the Brassica SRK sequences fall within the same clade as those of Arabidopsis and its relatives, despite the significantly greater phylogenetic distance between the genera as compared to Leavenworthia and Arabidopsis. By contrast, the Leavenworthia Lal2 sequences and their sequence homologs in other Brassicaceae taxa form a distinct clade, which appears to have diverged from the SRK-ARK clade before allelic diversification at SRK that presumably occurred at the onset of the ancestral SI system of Brassicaceae. As well, the Lal2 amino acid sequences have distinct deletions compared with those of Arabidopsis and Brassica SRKs. Finally, although the SCR-like gene in Leavenworthia shares several features in common with SCR, including high sequence diversity, a coding sequence with eight cysteine residues, and a defensin-like protein predicted to form a compact tertiary structure held together by disulfide bridges, they align too poorly with those of SCRs to be orthologous. Instead, the LaLal2 and LaSCRL sequences of Leavenworthia resemble SD-1 receptor kinase and defensin-like gene family members, respectively, found in a conserved syntenic block in A. lyrata, on the same chromosome as the SRK/SCR S locus but distant from it.

The Leavenworthia S locus appears to have evolved secondarily from paralogs of SRK and SCR. Without wishing to be bound to theory, below several possible explanations were proposed that could account for the distinct characteristics of the Leavenworthia S locus noted above. First the question of the time of the duplication event was addressed and gave rise to the separate SRK and Lal2 lineages, and second the question of the time of acquisition of pollen-pistil recognition function by Lal2/SCRL was addressed. Regarding the first issue, focusing on the phylogenetic relationships of the Lal2 and SRK sequences as shown in FIG. 2, it is noted that these two groups of sequences form separate clades, and that the Lal2 group belongs to a lineage that apparently diverged from the SRK group before SRK became involved in self-pollen recognition and underwent allelic diversification. The alternative hypothesis—that there was a duplication of SRK that gave rise to Lal2 and occurred while SRK was already functioning in self-incompatibility and thus still undergoing allelic diversification, but before the divergence of genera Arabidopsis, Capsella, Leavenworthia, and Brassica—is unlikely for the following reasons: (1) it is at odds with the structure of the gene tree and with the high level of divergence of Lal2 from SRK throughout the entire Lal2 sequence (Table 3); (2) under this hypothesis one would expect to find a gene tree with Lal2 and SRK sequences interspersed at the branch tips; and (3) if Lal2 functioned this early in SI as a pollen protein-receptor, one would expect the level of polymorphism at Lal2 to be high. In earlier work, it was shown that there is a relatively low level of polymorphism at LaLal2 compared with SRK, and evidence of strong positive selection in hypervariable regions of the S-domain thought to be involved in recognition was shown. Strong positive selection is thought to provide an indicator of recent diversification of the S locus, since negative-frequency dependent selection for new S-allele specificities is expected to be most pronounced when S allele numbers are low, as expected following recent evolution of an S locus, or a population bottleneck. Moreover, it was shown that the A. lyrata Lal2 and SCRL genes do not exhibit polymorphism.

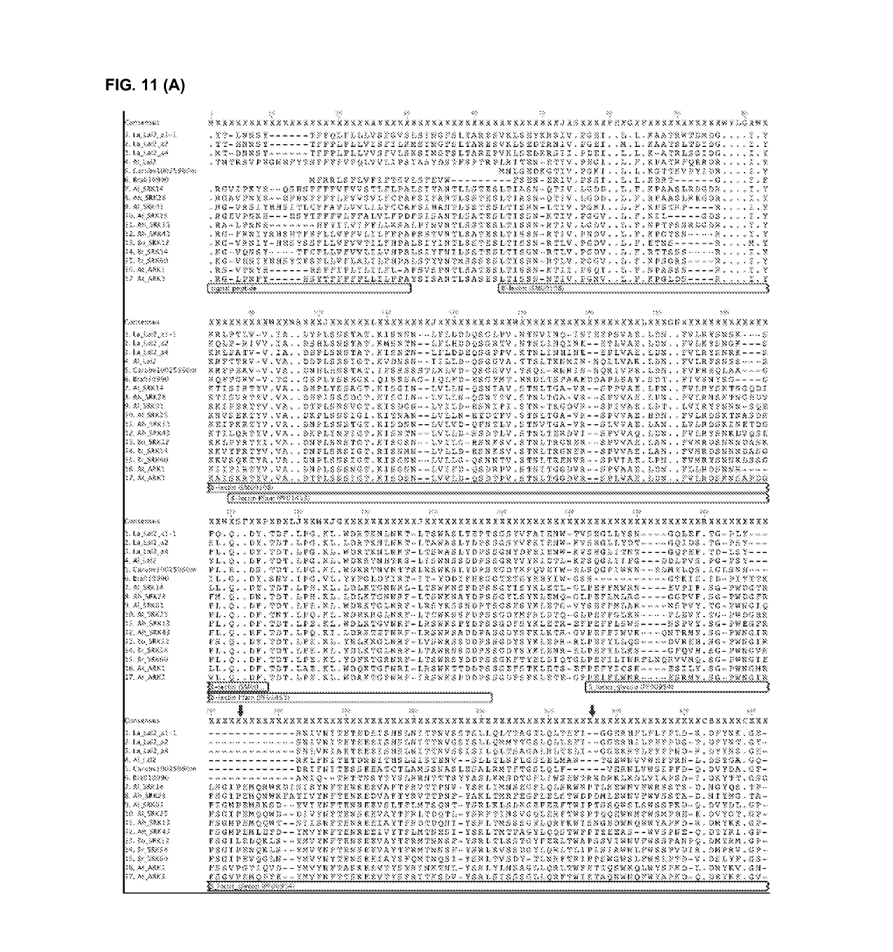

Regarding the issue of the time of acquisition of pollen-pistil recognition function by Lal2/SCRL, two alternative scenarios are proposed. In both cases it is assumed that divergence of SRK and Lal2 predates the origin of SI in the Brassicaceae, and moreover, at the time of origin of SI in the family, these two genes were paralogous, with distinct functions and genomic locations. It is assumed that the lineage leading to SRK then acquired a role in SI and subsequently diversified leading to a large clade of SRK alleles that exhibit transgeneric polymorphism. It also likely gave rise to related genes (that do not have a function in SI) through duplication and translocation to new genomic locations unlinked to the S locus, e.g., ARK1. According to the first scenario (Scenario I), the ancestral S locus (i.e. with SRK/SCR) was lost at some point in the lineage leading to Leavenworthia, and so functional SI was lost (FIG. 9). Pollen-pistil recognition then re-evolved based on a receptor-ligand system using the LaLal2 and LaSCRL genes, with a burst of diversification. Although this scenario involves a shift in the genes involved in pollen-pistil recognition in the SI system in the Leavenworthia lineage, it is possible that the genes involved in the signaling cascade leading to inhibition of pollen germination in the incompatibility reaction have remained the same as in the other lineages. Alternatively (Scenario II) the evolution of a new S locus in Leavenworthia could have been a two-step process, one in which SI was never completely lost (FIG. 9). This could have occurred if one gene of the new S locus (e.g., LaLal2) evolved pollen-protein recognition function, followed by evolution of a role as a protein ligand in SI for the second gene (LaSCRL), a series of events that could have been favored under high inbreeding depression if the ancestral system was “leaky” and allowed some selfing. Then, the original SRK/SCR S locus could have later been lost in Leavenworthia (perhaps following polyploidization). These two scenarios both fit the pattern of earlier divergence of Lal2 seen in the gene phylogeny (FIG. 2), and are compatible with the evidence of relatively low diversity of Lalal2 alleles, and detection of strong selection in hypervariable regions of LaLal2.

The data from this study are insufficient to know whether SI was lost in the lineage leading to Leavenworthia (Scenario I), or whether it was retained without interruption of the self-incompatibility response (Scenario II), but there are several reasons to consider that SI may have been lost in the Leavenworthia lineage before being regained. First, the loss of SI is indeed common in the flowering plants and in the Brassicaceae—it has been estimated that half the species in the family are self-compatible and thus, the possible loss of SI within Leavenworthia cannot be considered as an atypical event. Second, Leavenworthia has recently been shown to be a paleopolyploid species. As is the case in other such taxa, the evolutionary history of Leavenworthia likely involved interspecific hybridization followed by polyploidization. Hybridization and polyploidization in an individual possessing SI may lead to loss of fertility due to the absence of mates with gametes capable of producing viable offspring, which in turn could have led to selection for the loss of SI. That is, self-fertilization (as brought about by the loss of SI) may have increased the ability of an ancestral plant to form viable offspring—this is not to say that polyploidy must necessarily have led to the immediate breakdown of SI but rather that polyploidization could have provided a “selective filter” that favored its loss.

Clearly, Scenario I challenges the widely held notion that SI once lost is not easily regained. SI is however known to have evolved several times in the angiosperms, and so it is conceivable that it could re-evolve within the same family following loss of its pollen-pistil recognition system. It has been noted that the Brassicaceae is enriched for S-receptor kinase genes and these often occur near SCR-like genes. Given the role that these genes play in recognition, it is possible that they could have formed the basis of the pollen-pistil recognition system in SI more than once. As well, it was noted that, though not specific, the expression of Lal2 and SCRL in stigmas and anthers, respectively, in both A. lyrata and Leavenworthia suggest the presence of regulatory elements necessary to bring about a new S locus in the lineage leading to Leavenworthia.

It has been suggested that the loss of adaptations for outcrossing, and transition to a high self-fertilization rate represents an evolutionary dead end, either because selfing lineages have higher extinction rates than outcrossing ones (due to accumulation of deleterious mutations), because of loss of adaptability, or because once lost, the purging of the genetic load leads to reduced inbreeding depression, so that outcrossing mechanisms cannot be easily regained via selection. If the Lal2/SCRL S locus arose following the loss of SI, the re-evolution of SI would require that the selective pressure, inbreeding depression to be retained. Theory suggests that if inbreeding depression is largely due to mutations with low selective coefficients, and if moderate levels of outcrossing persist following loss of SI, inbreeding depression may not necessarily be purged.

Scenario II is also interesting to consider. It would likely entail a period of evolutionary history in the Leavenworthia lineage in which two separate S loci could have co-existed within the same genome. Self-incompatibility systems with two unlinked recognition loci are known in the grasses.

The Genetic Basic of SC in Leavenworthia.

Different disabling mutations at the SCR-like gene in different SC populations of L. alabamica were found, suggesting independent loss of SI in these populations. The same conclusion was also inferred based on phylogenetic relationships among the SI and SC populations of this species. The finding that mutations in the pollen gene are involved in each case where SI has been lost in L. alabamica parallels recent reports in Arabidopsis thaliana and A. kamchatica and also lends support to a prediction from population genetic theory that mutations disabling the pollen gene (as opposed to those disabling the stigma gene) should more easily spread in populations. Moreover, the loss of SI in L. alabamica was probably recent, as Lalal2 genes in the SC populations are apparently still intact and expressed, and at least one of the SC L. alabamica populations studied here (the a2 race population) exhibits mixed selfing and outcrossing. Had the loss of SI and breakdown of SCR-like genes in these populations occurred in the more distant evolutionary past, it would presumably have rendered the Lalal2 gene selectively neutral and subject to mutational decay, and we would have expected to find a signature of such decay or neutrality in LaLal2 sequences. However, he possibility that this gene also serves an additional unknown function cannot be ruled out, as suggested by the expression of LaLal2 in tissues other than stigmas. For example, a dual function has been found for an SRK gene in Arabidopsis.

### Example II

### Introduction of Leavenworthia Gene System into a Plant

Lal2 and SCRL were cloned along with, respectively, the stigma-specific promoter SLR1 of Brassica oleracea (Hackett et al., 1996) or its native promoter, and the anther-specific promoter ATA7 of Arabidopsis thaliana (Tsuchimatsu et al., 2010) or its native promoter, into the multiple cloning site of the plant transformation vector pORE O3 (Coutu et al., 2007) to produce six molecular constructs presented in Table 6. The SLR1 pro/pORE 03 construct originated from Chapman 2010 and was used to clone the Lal2 sequences. All DNA fragments were amplified by PCR using the Platinum Taq DNA Polymerase HiFi (Life Technologies) and restriction digests were carried using restriction enzymes from New England Biolabs. PCR products were purified on-column (Qiagen, QIAquick PCR Purification Kit). The gene constructs were transferred into Camelina sativa via Agrobacterium using the published floral dip transformation protocol of Liu et al. 2012. C. sativa transformed lines were produced separately for Lal2 and SCRL. The transformed lines were selected on 05× MS agar medium supplement with 15 μg/ml glufosinate ammonium (Sigma) and were transferred to soil.

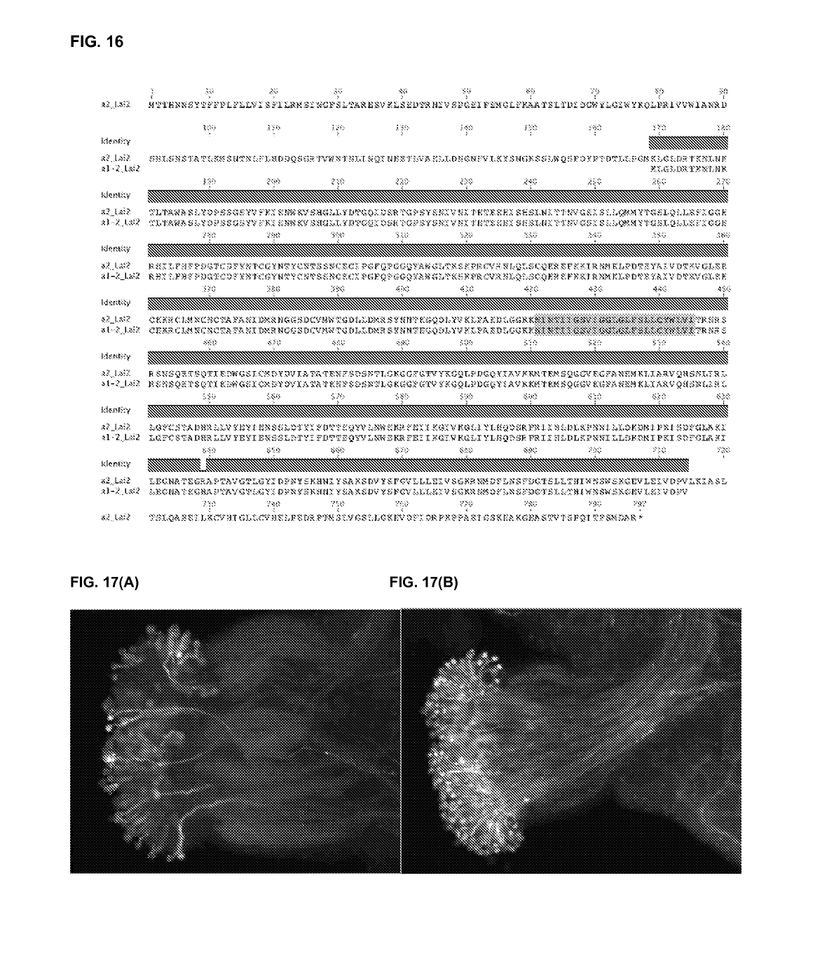

Hemizygous transformants of C. sativa made using the Lal2 and SCRL alleles of the same haplotype (a1-1 or a1-2) were crossed in all pairwise combinations (see Table 7). Self-pollen rejection phenotype was characterized in these lines by manually crossing the Lal2 transgenic plants with pollen from the SCRL transgenic plants and testing for pollen rejection. Pollen rejection was determined using microscopy analysis of pollen tube growth in pistils harvested 16 hours after manual pollination followed by aniline blue staining of the pistils (i.e., by fixing, clearing, and staining pollinated stigmas and counting pollen tubes that penetrated the stigma). Each cross was replicated 5 times. A pollen rejection reaction was scored when less than ten pollen tubes were observed in the pistil (FIG. 17).

TABLE 6
Primers and restriction sites used to generate constructs in the pORE O3 vector in Example II. Sequence of forward and reverse
primers is shown in Table 8.

| | | | Size of | Restriction | | SEQ | | SEQ |
|---|---|---|---|---|---|---|---|---|
| | DNA | | DNA | sites used for | | ID | | ID |
| Construct | amplified | DNA source | amplified | cloning | Forward primer | NO | Reverse primer | NO |
| 1. SLR1::a1- | a1-1 Lal2 | Leavenworthia | 2409 bp | Xmal-Notl | LaLal2-5prim_Xmal | 74 | LaLal2-3prim_Notl | 75 |
| 1Lal2 | cDNA | a1-1 stigmas | | | | | | |
| | | cDNA | | | | | | |
| 2. a1-1Lal2 | a1-1 Lal2 | Leavenworthia | 2912 bp | SacII-HindIII | Lal2_prom_SacII-F | 76 | Lal2_Sdomain3′-R | 77 |
| pro::Lal2 | pro + exon1 | a1-1 gDNA | | | | | | |
| 3. ATA7::a1- | a1-1 SCRL | Leavenworthia | 582 bp | Xmal-Notl | a1- | 78 | a1- | 79 |
| 1SCRL | gDNA | a1-1 gDNA | | | 1_SCRL_5prim_Xmal | | 1_SCRL_3prim_Notl | |
| | ATA7 | Arabidopsis Col-0 | 1998 bp | HindIII-Xmal | ATA7pro- | 80 | ATA7pro- | 81 |
| | promoter | gDNA | | | 5prim_HindIII | | 3prim_Xmal | |
| 4. a1-1 | a1-1 SCRL | Leavenworthia | 2656 bp | SacII-Xmal | SCRL_prom_SacII-F | 82 | SCRL_3UTR_Xmal-R | 83 |
| SCRLpro::SCRL | pro + gene | a1-1 gDNA | | | | | | |
| 5. SLR1pro::a1- | a1-2 Lal2 | Leavenworthia | 2391 bp | Xmal-Notl | Lal2_a2_Xmal-F | 84 | Lal2_a2_Notl-R | 85 |
| 2Lal2 | cDNA | a1-2 stigmas | | | | | | |
| | | cDNA | | | | | | |
| 6. ATA7pro::a1- | a1-2 SCRL | Leavenworthia | 600 bp | Xmal-Notl | a1- | 86 | a1- | 87 |
| 2 SCRL | gDNA | a1-2 gDNA | | | 2_SCRL_5prim_Xmal | | 2_SCRL_3prim_Notl | |
| | ATA7 | Arabidopsis Col-0 | 1998 bp | HindIII-Xmal | ATA7pro- | 80 | ATA7pro- | 81 |
| | promoter | gDNA | | | 5prim_HindIII | | 3prim_Xmal | |

TABLE 7
Pollen rejection reactions observed in crosses made between Lal2 and
SCRL transformants of Camelina sativa.

| | | | Pollen |
|---|---|---|---|
| | | Number of | rejection |
| Female parent line | Male parent line | crosses | reactions |
| (Lal2 transformant) | (SCRL transformant) | performed | observed |
| Line 1-15 | Line 3-13 | 5 | 3 |
| Line 1-15 | Line 4-21 | 5 | 3 |
| Line 1-25 | Line 3-13 | 5 | 4 |
| Line 1-25 | Line 4-21 | 5 | 1 |

TABLE 8
Nucleic acid sequence of forward
and reverse primers shown in Table 6.

| SEQ | | Nucleic acid |
|---|---|---|
| ID NO | Description | sequence |
| 74 | LaLaI2-5prim_XmaI | CCCGGGATGACGACTCT |
| | | CAACAATTCTTAC |
| 75 | LaLaI2-3prim_NotI | GCGGCCGCTCATCGAGC |
| | | GCCCATGGTG |
| 76 | LaI2_prom_SacII-F | CTGACCGCGGATGTTGA |
| | | ACATGTTCTGATG |
| 77 | LaI2_Sdomain3′-R | GAATTGCAACTGTACAG |
| | | CATTTGC |
| 78 | a1-1_SCRL_5prim_XmaI | CTGACCCGGGATGGCCA |
| | | AAAGTGTATGGCT |
| 79 | a1-1_SCRL_3prim_NotI | CTGAGCGGCCGCTTATT |
| | | TAAATGGAAACATGAG |
| 80 | ATA7pro-5prim_HindIII | AGTCAAGCTTAGTCTTC |
| | | TTGTACACGTCGAC |
| 81 | ATA7pro-3prim_XmaI | CTGACCCGGGGGCTTAG |
| | | TTTAATGAACACATG |
| 82 | SCRL_prom_SacII-F | CTGACCGCGGTAACCAT |
| | | GGCCATGAATTGC |
| 83 | SCRL_3UTR_XmaI-R | CTGACCCGGGTATCTCC |
| | | TTCCAAATAGTTC |
| 84 | LaI2_a2_XmaI-F | CTGACCCGGGATGACGA |
| | | CTCACAACAATTC |
| 85 | LaI2_a2_NotI-R | TGAGCGGCCGCTCAACG |
| | | AGCATCCATGGAG |
| 86 | a1-2_SCRL_5prim_XmaI | CTGACCCGGGATGGCCA |
| | | AAAGTGTATGGCT |
| 87 | a1-2_SCRL_3prim_NotI | CTGAGCGGCCGCTTATT |
| | | TAAATGGAAACATGAG |

Transformed lines (T1) are tested for the number of transgene insertion sites by segregation analysis of T1 progeny. Lines with transgene insertion in a single locus are tested for transgene expression in the appropriate tissue Lal2 in the stigmas and SCRL in the anthers) by RT-PCR. The thermal stability of SI phenotype is further analyzed in temperature-controlled growth chambers at several different temperatures. Lal2 and SCRL homozygous T2 plants are crossed to generate Lal2/SCRL doubly transformed T2 plants that are used as recipient parents of F1 hybrids and synthetic lines.

While the invention has been described in connection with specific embodiments thereof, it will be understood that the scope of the claims should not be limited by the preferred embodiments set forth in the examples, but should be given the broadest interpretation consistent with the description as a whole.

### REFERENCES

WO2011/034945
Boisvert S, Laviolette F, Corbeil J (2010) Ray: simultaneous assembly of reads from a mix of high-throughput sequencing technologies. J Comput Biol 17: 1519-1533. doi:10.1089/cmb.2009.0238.
Busch J W, Joly S, Schoen D J (2010) Does mate limitation in self-incompatible species promote the evolution of selfing? The case of Leavenworthia alabamica. Evolution 64: 1657-1670. doi:10.1111/j.1558-5646.2009.00925.x.
Busch J W, Sharma J, Schoen D J (2008) Molecular characterization of Lal2, an SRK-Like gene linked to the S-Locus in the wild mustard Leavenworthia alabamica. Genetics 178: 2055-2067. doi:10.1534/genetics.107.083204.
Carafa A, Carratu G (1997) Stigma treatment with saline solutions: a new method to overcome self-incompatibility in Brassica oleracea L. J Hortic Sci v. 72(4) p. 531-535.
Chantha, S-C, Herman, A C, Platts, A, Vekemans, X, Schoen, D J (2013) Secondary evolution of a self-incompatibility locus in the Brassicaceae genus Leavenworthia. PLoS Biology 11: e1001560.
Clough S J, Bent A F (1998) Floral dip: a simplified method for Agrobacterium-mediated transformation of Arabidopsis thaliana. Plant Jour 16:735-43.
Coutu C, Brandle J, Brown D, Brown K, Miki B, Simmonds J, et al. pORE: a modular binary vector series suited for both monocot and dicot plant transformation. Transgenic Res. 2007; 16: 771-781. doi:10.1007/s11248-007-9066-2
Chapman L. The Role of Sec15b and Phosphatidylinositol-4-Phosphate in Early Compatible Pollen-pistil Interactions. Thesis. 2010.
Darling A E, Mau B, Perna N T (2010) progressiveMauve: multiple genome alignment with gene gain, loss and rearrangement. PLoS ONE 5: e11147. doi:10.1371/journal.pone.0011147.
Drummond A J, Ashton B, Buxton S, Cheung M, Cooper A (2011) Geneious Pro. Available: http://www.geneious.com/.
Elsaesser R, Paysan J (2004) Liquid gel amplification of complex plasmid libraries. BioTechniques 37: 200-202.
Frazer K A, Pachter L, Poliakov A, Rubin E M, Dubchak I (2004) VISTA:
computational tools for comparative genomics. Nucleic Acids Res 32: W273-W279. doi:10.1093/nar/gkh458.
Gan X, Stegle O, Behr J, Steffen J G, Drewe P (2011) Multiple reference genomes and transcriptomes for Arabidopsis thaliana. Nature 477: 419-423. doi:10.1038/nature10414.
Gasser C S, Budelier K A, Smith A G, Shah D M, Fraley R T (1989) Isolation of Tissue-Specific cDNAs from Tomato Pistils. Plant Cell 1, 5-24.
Goldman M H, Goldberg R B, Mariani C (1994) Female sterile tobacco plants are produced by stigma-specific cell ablation. EMBO Journal 13, 2976-84.
Guindon S, Gascuel O (2003) A simple, fast, and accurate algorithm to estimate large phylogenies by maximum likelihood. Syst Biol 52: 696-704. doi:10.1080/10635150390235520.
Hackett R M, Cadwallader G, Franklin F C (1996) Plant physiology, 112(4):1601-7.
Hanks S, Quinn A, Hunter T (1988) The protein kinase family: conserved features and deduced phylogeny of the catalytic domains. Science 241: 42-52. doi:10.1126/science.3291115.
Harris R S (2007) Improved pairwise alignment of genomic DNA. Ph.D. thesis.
Haudry A, Zha H G, Stift M, Mable B K (2012) Disentangling the effects of breakdown of self-incompatibility and transition to selfing in North American Arabidopsis lyrata. Molecular Ecology 21: 1130-1142. doi:10.1111/j.1365-294X.2011.05435.x.
Herman A C, Busch J W, Schoen D J (2012) Phylogeny of Leavenworthia S-alleles suggests unidirectional mating system evolution and enhanced positive selection following an ancient population bottleneck. Evolution 66: 1849-1861. doi:10.1111/j.1558-5646.2011.01564.x.
Hrvatin S, Piel J (2007) Rapid isolation of rare clones from highly complex DNA libraries by PCR analysis of liquid gel pools. J Microbiol Methods 68: 434-436. doi:10.1016/j.mimet.2006.09.009.
Huelsenbeck J P, Ronquist F (2001) MRBAYES: Bayesian inference of phylogenetic trees. Bioinformatics 17: 754-755. doi:10.1093/bioinformatics/17.8.754.
KivimäKi M, KäRkkälnen K, Gaudeul M, LøE G, AGren J (2007) Gene, phenotype and function: GLABROUS1 and resistance to herbivory in natural populations of Arabidopsis lyrata. Molecular Ecology 16: 453-462. doi:10.1111/j.1365-294X.2007.03109.x.
Kuhn R M, Haussler D, Kent W J (2012) The UCSC genome browser and associated tools. Brief Bioinform. doi:10.1093/bib/bbs038.
Letunic I, Doerks T, Bork P (2011) SMART 7: recent updates to the protein domain annotation resource. Nucleic Acids Res 40: D302-D305. doi:10.1093/nar/gkr931.
Liu X, Brost J, Hutcheon C, Guilfoil R, Wilson A K, Leung S, Shewmaker C K, Rooke S, Nguyen T, Kiser J, De Rocher J (2012) Transformation of the oilseed crop Camelina sativa by Agrobacterium-mediated floral dip and simple large-scale screening of transformants. In vitro cell dev biol-Plant 48:462-468.
Lu C, Kang J (2008) Generation of transgenic plants of a potential oilseed crop Camelina saliva by Agrobacterium-mediated transformation. Plant Cell Report 27:273-78.
Lyons-Weiler J, Hoelzer G A, Tausch R J (1998) Optimal outgroup analysis. Biol J Linn Soc Lond 64: 493-511. doi:10.1111/j.1095-8312.1998.tb00346.x.
McClur B A, Haring V, Ebert P R, Anderson M A, Simpson R J, Sakiyama F, Clarke A E (1989) Style self-incompatibility gene products of Nicotiana alata are ribonucleases. Nature 342, 955-957)
Sievers F, Wilm A, Dineen D, Gibson T J, Karplus K (2011) Fast, scalable generation of high-quality protein multiple sequence alignments using Clustal Omega. Mol Syst Biol 7. doi:10.1038/msb.2011.75.
Yang Z (2007) PAML 4: Phylogenetic Analysis by Maximum Likelihood. Mol Biol Evol 24: 1586-1591. doi:10.1093/molbev/msm088.
Zhang J, Nielsen R, Yang Z (2005) Evaluation of an improved branch-site likelihood method for detecting positive selection at the molecular level. Mol Biol Evol 22: 2472-2479. doi:10.1093/molbev/msi237.
Zhang X, Wang L, Yuan Y, Tian D, Yang S (2011) Rapid copy number expansion and recent recruitment of domains in S-receptor kinase-like genes contribute to the origin of self-incompatibility. FEBS Journal 278: 4323-4337. doi:10.1111/j.1742-4658.2011.08349.x.

What is claimed is:

1. A first isolated nucleic acid molecule encoding for a Lal2 polypeptide, wherein the Lal2 polypeptide is capable of intracellular signaling upon specifically binding to a SCRL polypeptide and is at least one of:
(i) a polypeptide having the amino acid sequence of SEQ ID NO: 66,
(ii) a polypeptide encoded by a Lal2 gene ortholog, and
(iii) a variant polypeptide of the polypeptide of (i) or (ii)
wherein the SCRL polypeptide is derived from a SCRL gene located within 10 000 bp of a corresponding Lal2 gene.

2. The first isolated nucleic acid molecule of claim 1 being a complementary DNA (cDNA.)

3. The first isolated nucleic acid molecule of claim 1, wherein the polypeptide of (i) has at least one cysteine residue at positions corresponding to amino acid residues 283, 289, 295, 301, 303, 324, 332, 362, 366, 370, 372 or 387 of SEQ ID NO: 66.

4. The first isolated nucleic acid molecule of claim 1, wherein the polypeptide of (i) has the amino acid sequence of any one of SEQ ID NO: 5 to 7.

5. A first vector comprising a promoter operatively linked to a first transgene encoding a transgenic Lal2 polypeptide, wherein the first transgene comprises the first isolated nucleic acid molecule of claim 1.

6. The first vector of claim 5, wherein the promoter is a stigma-specific or a stigma-active promoter.

7. A first transgenic Agrobacterium host cell, a first transgenic Brassicaceae plant or a first transgenic Brassicaceae cell comprising the first vector of claim 5.

8. A second isolated nucleic acid molecule encoding for a SCRL polypeptide, wherein the SCRL polypeptide is capable of specifically binding to a Lal2 polypeptide so as to allow the Lal2 polypeptide to mediate intracellular signaling and is at least one of:
(i) a polypeptide having the amino acid sequence of SEQ ID NO: 72;
(ii) a polypeptide encoded by a SCRL gene ortholog; and
(iii) a variant polypeptide of the polypeptide of (i) or (ii);
wherein the SCRL polypeptide is derived from a SCRL gene located within 10 000 bp of a corresponding Lal2 gene.

9. The second isolated nucleic acid molecule of claim 8 being a complementary DNA (cDNA).

10. The second isolated nucleic acid molecule of claim 8, wherein the polypeptide of (i) has at least one cysteine residue residues at positions corresponding to amino acid residues 56, 65, 69, 80, 89, 91, and 97 of SEQ ID NO: 72.

11. The second isolated nucleic acid molecule of claim 8, wherein the polypeptide of (i) has the amino acid sequence of any one of SEQ ID NO: 1 to 2.

12. A second vector comprising a promoter operatively linked to a second transgene encoding a transgenic SCRL polypeptide, wherein the second transgene comprises the second isolated nucleic acid molecule of claim 8.

13. The second vector of claim 12, wherein the promoter is an anther tapetum-specific or an anther tapetum-active promoter.

14. A second transgenic Agrobacterium host cell, a second transgenic Brassicaceae plant or a second transgenic Brassicaceae cell comprising the second vector of claim 12.

15. A method for producing a self-incompatible transgenic Brassicaceae plant, said method comprising (a) crossing the first transgenic Brassicaceae plant claim 8 with the second transgenic Brassicaceae plant of claim 14 so as to obtain a crossed transgenic Brassicaceae and (b) identifying the crossed transgenic Brassicaceae as being self-incompatible if the crossed Brassicaceae plant is a double-transgenic for the first transgene and the second transgene.

16. A self-incompatible transgenic Brassicaceae plant having (i) a first transgene comprising the first isolated nucleic acid molecule of claim 1 and (ii) a second transgene comprising the second isolated nucleic acid molecule of claim 8 and (ii) being a double-transgenic for the first transgene and the second transgene.

17. The self-incompatible transgenic Brassicaceae plant of claim 16 being a Camelina plant.

18. A genetic system for producing a self-incompatible Brassicaceae plant, said genetic system comprising:
at least one of the first isolated nucleic acid of claim 1, the first vector of claim 5, the first transgenic Agrobacterium host cell, the first transgenic Brassicaceae plant or the first transgenic Brassicacea cell of claim 7; and
at least one of the second isolated nucleic acid of claim 8, the second vector of claim 12, the second transgenic Agrobacterium host cell, the second transgenic Brassicaceae plant or the second transgenic Brassicacea cell of claim 14.

19. A method for producing an hybrid Brassicaceae plant or cell, said method comprising (a) crossing the self-incompatible transgenic Brassicaceae plant of claim 16 with a second Brassicaceae plant so as to provide a crossed Brassicaceae plant and (b) identifying the crossed Brassicaceae plant as the hybrid Brassicaceae plant or cell if the crossed Brassicaceae exhibits a first trait unique to the self-incompatible transgenic Brassicaceae plant and a first trait unique to the second Brassicaceae plant.

20. A hybrid Brassicaceae plant or cell hemizygous for a first transgenic nucleic acid molecule as defined in claim 1 and for a second transgenic nucleic acid molecule as defined in claim 8.
